SEQUENCE LISTING (80 sequences)

SEQ ID NO: 1 - DNA, 24 bp, Artificial Sequence
  Ps#1sgRNA F
attggaatgtctgttgccttgtta

SEQ ID NO: 2 - DNA, 23 bp, Artificial Sequence
  Ps#1 sgRNA R
aaactaacaaggcaacagacatt

SEQ ID NO: 3 - DNA, 24 bp, Artificial Sequence
  Ps#2 sgRNA F
attggagcgtatataatgctgctt

SEQ ID NO: 4 - DNA, 23 bp, Artificial Sequence
  Ps#2 sgRNA R
aaacaagcagcattatatacgct

SEQ ID NO: 5 - DNA, 24 bp, Artificial Sequence
  a 3756 bic hr f
tcagctatgctaatgactcccgag

SEQ ID NO: 6 - DNA, 26 bp, Artificial Sequence
  a bic hr r
agtccattctctattccgcatagtga

SEQ ID NO: 7 - DNA, 24 bp, Artificial Sequence
  a 3756 r
tgacaaccgacctaaatcgatccg

SEQ ID NO: 8 - DNA, 26 bp, Artificial Sequence
  b bic hr r
actgcattctctattccgcatagtga

SEQ ID NO: 9 - DNA, 28 bp, Artificial Sequence
  b 3756 r
gactactgaaccgacctaaatcgatccg

SEQ ID NO: 10 - DNA, 34 bp, Artificial Sequence
  pupd_y1_f
gcgccgtctcgctcgtactcgaacgagggtcatc

SEQ ID NO: 11 - DNA, 41 bp, Artificial Sequence
  pupd_y1_r
gcgccgtctcgctcgagcgccataattggaacactcatcaa

SEQ ID NO: 12 - DNA, 37 bp, Artificial Sequence
  pupd_ps_f
gcgccgtctcgctcgggagcaaccttattttgtactt

SEQ ID NO: 13 - DNA, 39 bp, Artificial Sequence
  pupd_ps_r
gcgccgtctcgctcgagtacaacatatcaaaataggtat

SEQ ID NO: 14 - DNA, 20 bp, Artificial Sequence
  psy1 t1 htp f
ggtttgcctgtctgtggtct

SEQ ID NO: 15 - DNA, 25 bp, Artificial Sequence
  psy1 t1 htp r1
agtcccatgaaacttgtcccatttg

SEQ ID NO: 16 - DNA, 25 bp, Artificial Sequence
  psy1 t1 htp r2
tcagccatgaaacttgtcccatttg

SEQ ID NO: 17 - DNA, 25 bp, Artificial Sequence
  psy1 t1 htp r3
actgccatgaaacttgtcccatttg

SEQ ID NO: 18 - DNA, 25 bp, Artificial Sequence
  psy1 t1 htp r4
tgacccatgaaacttgtcccatttg

SEQ ID NO: 19 - DNA, 25 bp, Artificial Sequence
  psy1 t1 htp r5
gactccatgaaacttgtcccatttg

SEQ ID NO: 20 - DNA, 25 bp, Artificial Sequence
  psy1 t1 htp r6
ctgaccatgaaacttgtcccatttg

SEQ ID NO: 21 - DNA, 20 bp, Artificial Sequence
  nhej_psy1_t2_r
gcctaaatacggcacttcca

SEQ ID NO: 22 - DNA, 24 bp, Artificial Sequence
  a_nhej_psy1_t2_f
agtcgtatcgcccctgaatcaaag

SEQ ID NO: 23 - DNA, 24 bp, Artificial Sequence
  b_nhej_psy1_t2_f
tcaggtatcgcccctgaatcaaag

SEQ ID NO: 24 - DNA, 24 bp, Artificial Sequence
  c_nhej_psy1_t2_f
tgacgtatcgcccctgaatcaaag

SEQ ID NO: 25 - DNA, 24 bp, Artificial Sequence
  d_nhej_psy1_t2_f
actggtatcgcccctgaatcaaag

SEQ ID NO: 26 - DNA, 24 bp, Artificial Sequence
  e_nhej_psy1_t2_f
gactgtatcgcccctgaatcaaag

SEQ ID NO: 27 - DNA, 24 bp, Artificial Sequence
  f_nhej_psy1_t2_f
ctgagtatcgcccctgaatcaaag

SEQ ID NO: 28 - DNA, 30 bp, Artificial Sequence
  nnn_a_nhej_psy1_t2_f
  misc_feature (1)..(6): n is a, c, g, or t
nnnnnnagtcgtatcgcccctgaatcaaag

SEQ ID NO: 29 - DNA, 30 bp, Artificial Sequence
  nnn_b_nhej_psy1_t2_f
  misc_feature (1)..(6): n is a, c, g, or t
nnnnnntcaggtatcgcccctgaatcaaag

SEQ ID NO: 30 - DNA, 26 bp, Artificial Sequence
  nnn_nhej_psy1_t2_r
  misc_feature (1)..(6): n is a, c, g, or t
nnnnnngcctaaatacggcacttcca

SEQ ID NO: 31 - DNA, 22 bp, Artificial Sequence
  PSY1_1_F
tttgcagaagtcaagaaacagg

SEQ ID NO: 32 - DNA, 20 bp, Artificial Sequence
  PSY1_t4_ident_R
gatgtcatcgtccgttctcc

SEQ ID NO: 33 - DNA, 20 bp, Artificial Sequence
  PSY1 psnps F
acggtatcttcccaccttca

SEQ ID NO: 34 - DNA, 25 bp, Artificial Sequence
  PSY1 psnps R2
atagtgttaattgtgtaggctcctt

SEQ ID NO: 35 - DNA, 20 bp, Artificial Sequence
  PSY1 psnps F2
cgacgaggagtaaggtttgc

SEQ ID NO: 36 - DNA, 20 bp, Artificial Sequence
  PSY1 psnps R
tcagtccatttcgttttcgt

SEQ ID NO: 37 - DNA, 20 bp, Artificial Sequence
  psy1_t123_f
atgttgcagccattcagaga

SEQ ID NO: 38 - DNA, 20 bp, Artificial Sequence
  psy1_t123_r
tgatcatggctcgtcactgt

SEQ ID NO: 39 - DNA, 20 bp, Artificial Sequence
  psy1 term f
acaagtaccctgggttggag

SEQ ID NO: 40 - DNA, 21 bp, Artificial Sequence
  psy1_term_r2
gcagtttttgtaggaggcaca

SEQ ID NO: 41 - DNA, 21 bp, Artificial Sequence
  psy1_term_f2
tgtgcctcctacaaaaactgc

SEQ ID NO: 42 - DNA, 23 bp, Artificial Sequence
  psy1 term r
tggattgaatcgaatttggataa

SEQ ID NO: 43 - DNA, 21 bp, Artificial Sequence
  pimpixm82_co_f
ctttgcacttggttactcaga

SEQ ID NO: 44 - DNA, 20 bp, Artificial Sequence
  pb_psy1_r
agcctacggcccaaactatt

SEQ ID NO: 45 - DNA, 20 bp, Artificial Sequence
  14036_f
tgctaatggggcaggaaata

SEQ ID NO: 46 - DNA, 25 bp, Artificial Sequence
  14036_r
tcaagtaacgtaaaacacgttgaaa

SEQ ID NO: 47 - DNA, 20 bp, Artificial Sequence
  5kb_up_t2_F
ttcatttgacgagcgatctg

SEQ ID NO: 48 - DNA, 20 bp, Artificial Sequence
  5kb_up_t2_R
ttggctgctttgaccttacc

SEQ ID NO: 49 - DNA, 24 bp, Artificial Sequence
  40kb_down_t2_f
cattatcctaagagtgcagtcagc

SEQ ID NO: 50 - DNA, 23 bp, Artificial Sequence
  40kb_down_t2_r
tggtttctcgattacctctttca

SEQ ID NO: 51 - DNA, 20 bp, Artificial Sequence
  20kb_down_t2_f
tgacaccaatccatccaatc

SEQ ID NO: 52 - DNA, 20 bp, Artificial Sequence
  20kb_down_t2_r
ctgctacctgcactggctct

SEQ ID NO: 53 - DNA, 20 bp, Artificial Sequence
  20kb_up_t2_f
tacgtccccgaagaaatcac

SEQ ID NO: 54 - DNA, 20 bp, Artificial Sequence
  20kb_up_t2_r
cccttaggctccgaagttgt

SEQ ID NO: 55 - DNA, 24 bp, Artificial Sequence
  40kb_up_t2_f
cacataagaggacacgtttattca

SEQ ID NO: 56 - DNA, 20 bp, Artificial Sequence
  40kb_up_t2_r
gccacggagaaaatagttga

SEQ ID NO: 57 - DNA, 20 bp, Artificial Sequence
  JF_F
tgcaaagtgctacgtgtcct

SEQ ID NO: 58 - DNA, 20 bp, Artificial Sequence
  PSY1_1_R
aatgtgaacagcaacgcaaa

SEQ ID NO: 59 - DNA, 18063 bp, Artificial Sequence
  35SCas9 plasmid
ggggacaagtttgtacaaaaaagcaggctatggatccccgggatcatctacttctgaagactcagactcagactaagcaggtgacgaacgtcaccaatcccaattcgatctacatcgataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgaacccagctttcttgtacaaagtgggggttcgaaatcgataagcttggatcctctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatctgcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagggtcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatcgacctgcaggcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgaggaattcggtacccc

SEQ ID NO: 60 - DNA, 12150 bp, Artificial Sequence
  U6-26 promoter Ps#1 gRNA
acctagggagctctcccatatcgacctgcaggcggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagggtcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctccctaggctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgaatgtctgttgccttgttagttttagagctagaaatagcaagttaaaataaggctagtccgttatcaacttgaaaaagtggcaccgagtcggtgctttttttctagacccagctttcttgtacaaagttggcatt

SEQ ID NO: 61 - DNA, 104 bp, Artificial Sequence
  gRNA molecule fo Allele specificDSB+Alleledependent repair
ggagcgtatataatgctgcttgttttagagctagaaatagcaagttaaaataaggctagtccgttatcaacttgaaaaagtggcaccgagtcggtgcttttttt

SEQ ID NO: 62 - DNA, 13688 bp, Artificial Sequence
  R1 Plasmid
ggagcaaccttattttgtactttaaaaaattcattttttttattttttcgactttaaagccaaaattatcctttatttatgaaagtggatgtatttttatccctttaatataaggttgagttttctttaagtttgtaatgttaagtggggcactttatattagtccaattaacagaattgacttagaaattgatggcgattacttgaaaaaatcgctacaaaaacataataaaataaaatttcatttgatttttaaaccaaaagaaaagtgacatgactaacgatttgatcccatcttcttttttttttaagaaaaaaattaaaagaaaaatttattaaactggatgattaattttaacataaaaattaaaaaaataaaaatcaatcgagctcactatttgatattattattttcatcttgatattgttacttcatatcactgaattataaaatgcgttaacggcaattatcatagacgcagactgaaagcataaaattacaactattccatttttgttttttttttgccaacccaaaaaaaactaataatttatttacataattatagaaaattggaatttattcatgtttttggaccattcagataaccccacaataaaaaaaaagtgcacaaaagtgcaaagtgctacgtgtcctaattatagccatactacatttgtgcataaaattagccaccattatttttaagattattctttttcttgttttaatttatatgccacactttcttattaatccatcttaaacagaatgacacactttttatttgacaaatatttaatgacattattaacattttatcctcgttagatttcacttatttggttaaaggtggtgtacttttctaatctaaaatcattaattttaggtataattttagaacattgcaaagccttttcatatatttaaacaatgtacataattatgtaattatgttatgttatataaattgagacgaaaaaaatattatttatcaggtacgagtgtcactttacactagaggtgtttacggacagagacgaatccaagatttgaacttcatggtgtcaagtatacacattgcttagattattaaatcaactttaggtttcaattccgcacaaaatctataaaacataacctttttaaaaataattttactaatattataaacatatattattcatttataatagttctcgacgagtttacatattttcaaaactgtcaattagaacatcattacttatatttgtaaatgtatcacttactacgtaacaaactaaacaaatcatttgaatatcactcatcactcttatttatattttatttttttatctacttcaccaaagaaaatgataatttcacaattgcgattgctaccagaaaaatatttacttaatcaattttaaaaagattaagtaatagaaatattttttattttattaagaaaagaggattttatcatatctaaatataaattaaagatgaagttactatgatattatttaaaaagtaaatgacattattctatttaaattacaaaatgagattaagaaaagtataactatttccatttttttagctaaaaggatttcaaaaaaaaaagtaacaagaaagagattttttaaaaataataattttattagaaatatatataaaaaattaataatattcgttagaaaaagtgaattttcaaaataataaagtaataacacgaatacttttagggtcaaacaattaatagtggagaatggagatatttttagtacaaattatttatacaaaatatctttattcaatgtgacataattttaagctatttttaacacttttccatcaaataaataattaataacaaattattaaaaaatcaaagattaataaattaaattaatgaactataattagttattaaaaccaagccaactagaattaaatagaaaaaaggaaatataatcttcaaaatatgctttgttgaagtataattcctcgtctatttaaagcttcggagatttaaattggcaagcgaattacacaaagcaaatgaaactacaccttttggttctgtgggtgtcatttgcttatttataaacattgtacatatatacacctatatgtatatcacgtttcgatcgaagctcgtgggtggcgtggcacccataaatcgtagtagattcgcccctgtttacggatcgatttaggtcggttatagatcaaaatcaaatcaatttaattaattttaccaatcaaaccaaatcaaatatactatacatttattggtttggttgttcgaatttcgatttgatttgattcaattattcgattatcaatcatacagaaataaaagaaacaattcattttaaagaggaacaaaagaggcaagaactcattcaaaatgaaaaatacttaaaatagccgaatttataaaactttttattactaaatttactgtgaataaaacttttaaattcaccattttagcctagaagtaagagagaaaaacatttccaccaagaattgtcaagaatactcatatacatgaaaccaatatgataaatgttctcttcaggacggttcataaataaattatgataaacatatataagatatttaaaataaaataaaatatatatatataaaatttatcggttcgattatttcttcaactttttaaataaaatcaaagactatcaattcttaagaatcttagaacaaactaaaccaaataaaattaaacgatttaatcaacttaatttaatttttcaatgtgggtcaatttttatccaaatacttttacacccacttttcaccatcacaaggggtaagaactagcaaattattttgccttaacaaaaacttactattggtgtagagaaaattggttgattttcttatcaatatatataatttagtacccaattttcccaaaactaaataaaatatagaatatccactaaggtggttacacgtgtacactcaaccaatgacggcacttcaaattcttgataacggtatcttcccaccttcatatatttcaacattttattgttaaaataaaatcgcacgctctattttatctaaattttatttatagaattatattaagtatgttattattaatttttacgtataaatatatttcatattaaaatcatataagttgacatcactaagttcgttggatttgcatgtagacccaattattcagatactcaacatgactcatttaatattggattattgaaatatttgtaatacattaagaatataattgttaactattttaatttttaattatacgtaaatatcaaacaaaaatattaatatgatcgctatattagatgataactataaggagcctacacaattaacactatttaactctattctttgcatttataaaaagttactttagtcttaggttcacaatgtcaaaatctaaacaactaaaaacgacgaggagtaaggtttgcaacgacgataacaaggattaggcaacaattagagttgtgaattgtgagtattaactatacttttactatattaggcagaatttttgcactcaatgagtaacttgatttatttattttttatttcgccctaaattattggacaagtcatatatttgttttgaaaacattcttttattggctaaatcgaaaattgaatcgttaaagatcaaaaatcaataacaaatatcttattggtttaacatatttaaaaataaaaaaccaataaatctaactaataatatttaatacgaaaacgaaatggactgacacacattcctaaatttttggtcaaaattttttcataatttccctaaaatctaaaatattaaatatttgacggaaacaaaaaattcacttttaataaattatttgaaggactaaaacagtggaagaatatatttaagaagctaatttgaacctagtgccaaatataaagggaccatttttgtcatttttcaacttgaaaatctacgtgtcttaatataacaccaaagaattaatatttactgaaaaaatgtaaaaatgaggatatggattctgaatcactcaattccaatcagcaaaaataaaataaaataaaataaaataaaatttaaaaaataataataaatgctataaaatgaccaaaatgtgtggagcaaaaagtgcagaaaaaaccaacaaattgcattctccattcttggaagtggccattcttgatttcttgaaacaaaggtttgtttcccttcacttcttgatatgtaaagttgcaatctttataactttctattgctttgctagtgtttttgttatatacagggggtggagttagagggtaagttacgcatttagtcgtaactttagtcaaacttcgtaataatttagtaagttaaaatatattagaaattttcagaattcataaactttaaattttaaattttgacttcgctttgtgtgactatacaattacagaaattcagagtggccattgttgaaagagagggtggaatttgtgtaagttttgtttcctttcagttcttgatatataaagttgcaatctttaacattctttgttcactttctataggtttgctaggttcggttaaattcagtagctttagtttaaaccctatgcggaatagagaatgtgtaaactttaaacttcaaattttggctccgcatacgactagcgactatataataataggaattgagcacttggcttttgtatatagcttctatgtgtaccaaaattagaaaatcaggcgattattataatcttgttgactaaatatagaatgcatccattacccccaaaaagtgtgattccactgtcataggaggttttttttatttcattttatttgtgctttcaataatgtagagtagttttacaaagatcctttctttgtgacacatggtaggtaatattgctgattttgttgtagttttggggttataaagtttcaaattatttatactggagggtaggggtgggggttgtctataatgcaggttatggttttacgtgaactcaataattattgtagatactaagaaatccactcagtgttcttgcggtgtcttgcttttgatttcagcatcacttgtagttgattgtgtttagattatcacattattctgtggctgtaactgtatccttgttagttgctttgtttctacactgttgttttccctcttttatacctattttgatatgttgtactcgaacgagggtcatcggggaacaacctctttacctccgtgaggtagagctatggtctgtgtccactctaccctccccagatccctcttgtaggatttcactatattgtaatattaacttgaggtcactataggagctcaaaaacttctaattttgaatcaatgtctggttatactttttttgtcataactgtatctcaaatgtggtgtttggtttatctcattttgcagaagtcaagaaacaggttactcctgtttgagtgaggaaaagttggtttgcctgtctgtggtctttttataatctttttctacagaagagaaagtgggtaattttgtttgagagtggaaatattctctagtgggaatctactaggagtaatttattttctataaactaagtaaagtttggaaggtgacaaaaagaaagacaaaaatcttggaattgttttagacaaccaaggttttcttgctcagaatgtctgttgccttgttatgggttgtttctccttgtgacgtctcaaatgggacaagtttcatggaatcagtccgggagggaaaccgtttttttgattcatcgaggcataggaatttggtgtccaatgagagaatcaatagaggtggtggaaagcaaactaataatggacggaaattttctgtacggtctgctattttggctactccatctggagaacggacgatgacatcggaacagatggtctatgatgtggttttgaggcaggcagccttggtgaagaggcaactgagatctaccaatgagttagaagtgaagccggatatacctattccggggaatttgggcttgttgagtgaagcatatgataggtgtggtgaagtatgtgcagagtatgcaaagacgtttaacttaggttagcttcttcaatctattcattcgtttaccaaatattatttggtaagcactaattatgaatatatatatgttcatgttattgatgaagacaaaatttgatctttgtttgtttattcaggaactatgctaatgactcccgagagaagaagggctatctgggcaatatatggtgaggtttctagccatttaataacagttacgcgcacaaacacatatgattaatcggggacgagaaaaaaagaaatgaagtttgagttttgagggtcatatgtaataggtaaatccgagcttgactagcttgagatgtttattgtcatatcatgctcaatactaaccaaaacactgaaaaagaacttgattatatttacatactaatattttcatttgcgttgctgttcacatttttacctatggaactggtttttgtgatttgttatacttcatattcgatgttaataaaatatatcattcctccctttttctccacttcaagctttactgtagtgttgaaaggggaaactccttttaatgattgcatatataaacgaacttcttgagttgaatagtttctcattatgatctgtttaaacagtatggtgcagaagaacagatgaacttgttgatggcccaaacgcatcatatattaccccggcagccttagataggtgggaaaataggctagaagatgttttcaatgggcggccatttgacatgctcgatggtgctttgtccgatacagtttctaactttccagttgatattcaggttagtctaccaattctatggtctttatatttgttcaatttgcgtttgatgtcacttttgctgagggcttttctaatagcttacttcagcctagcggaaatgtttgtagttgaatctctagttctgtctcctatatctgtttctctcgtcctagatactacacatacttcatttctgttttaacattttattcgtcttttggtgttgttttgtatgtgaatcatatatttggaacagaatcattattagttcacatgatttcatttgctttcttcaatagcgtaattgtctaaccttccaatatatgttgcagccattcagagatatgattgaaggaatgcgtatggacttgagaaaatcgagatacaaaaacttcgacgaactatacctttattgttattatgttgctggtacggttgggttgatgagtgttccaattatggcgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctca

SEQ ID NO: 63 - DNA, 9902 bp, Artificial Sequence
  R2 Plasmid
ggagcaaccttattttgtactttaaaaaattcattttttttattttttcgactttaaagccaaaattatcctttatttatgaaagtggatgtatttttatccctttaatataaggttgagttttctttaagtttgtaatgttaagtggggcactttatattagtccaattaacagaattgacttagaaattgatggcgattacttgaaaaaatcgctacaaaaacataataaaataaaatttcatttgatttttaaaccaaaagaaaagtgacatgactaacgatttgatcccatcttcttttttttttaagaaaaaaattaaaagaaaaatttattaaactggatgattaattttaacataaaaattaaaaaaataaaaatcaatcgagctcactatttgatattattattttcatcttgatattgttacttcatatcactgaattataaaatgcgttaacggcaattatcatagacgcagactgaaagcataaaattacaactattccatttttgttttttttttgccaacccaaaaaaaactaataatttatttacataattatagaaaattggaatttattcatgtttttggaccattcagataaccccacaataaaaaaaaagtgcacaaaagtgcaaagtgctacgtgtcctaattatagccatactacatttgtgcataaaattagccaccattatttttaagattattctttttcttgttttaatttatatgccacactttcttattaatccatcttaaacagaatgacacactttttatttgacaaatatttaatgacattattaacattttatcctcgttagatttcactatgcggaatagagaatgtgtaaactttaaacttcaaattttggctccgcatacgactagcgactatataataataggaattgagcacttggcttttgtatatagcttctatgtgtaccaaaattagaaaatcaggcgattattataatcttgttgactaaatatagaatgcatccattacccccaaaaagtgtgattccactgtcataggaggttttttttatttcattttatttgtgctttcaataatgtagagtagttttacaaagatcctttctttgtgacacatggtaggtaatattgctgattttgttgtagttttggggttataaagtttcaaattatttatactggagggtaggggtgggggttgtctataatgcaggttatggttttacgtgaactcaataattattgtagatactaagaaatccactcagtgttcttgcggtgtcttgcttttgatttcagcatcacttgtagttgattgtgtttagattatcacattattctgtggctgtaactgtatccttgttagttgctttgtttctacactgttgttttccctcttttatacctattttgatatgttgtactcgaacgagggtcatcggggaacaacctctttacctccgtgaggtagagctatggtctgtgtccactctaccctccccagatccctcttgtaggatttcactatattgtaatattaacttgaggtcactataggagctcaaaaacttctaattttgaatcaatgtctggttatactttttttgtcataactgtatctcaaatgtggtgtttggtttatctcattttgcagaagtcaagaaacaggttactcctgtttgagtgaggaaaagttggtttgcctgtctgtggtctttttataatctttttctacagaagagaaagtgggtaattttgtttgagagtggaaatattctctagtgggaatctactaggagtaatttattttctataaactaagtaaagtttggaaggtgacaaaaagaaagacaaaaatcttggaattgttttagacaaccaaggttttcttgctcagaatgtctgttgccttgttatgggttgtttctccttgtgacgtctcaaatgggacaagtttcatggaatcagtccgggagggaaaccgtttttttgattcatcgaggcataggaatttggtgtccaatgagagaatcaatagaggtggtggaaagcaaactaataatggacggaaattttctgtacggtctgctattttggctactccatctggagaacggacgatgacatcggaacagatggtctatgatgtggttttgaggcaggcagccttggtgaagaggcaactgagatctaccaatgagttagaagtgaagccggatatacctattccggggaatttgggcttgttgagtgaagcatatgataggtgtggtgaagtatgtgcagagtatgcaaagacgtttaacttaggttagcttcttcaatctattcattcgtttaccaaatattatttggtaagcactaattatgaatatatatatgttcatgttattgatgaagacaaaatttgatctttgtttgtttattcaggaactatgctaatgactcccgagagaagaagggctatctaggcaatatatggtgaggtttctagccatttaataacagttacgcgcacaaacacatatgattaatcggggacgagaaaaaaagaaatgaagtttgagttttgagggtcatatgtaataggtaaatccgagcttgactagcttgagatgtttattgtcatatcatgctcaatactaaccaaaacactgaaaaagaacttgattatatttacatactaatattttcatttgcgttgctgttcacatttttacctatggaactggtttttgtgatttgttatacttcatattcgatgttaataaaatatatcattcctccctttttctccacttcaagctttactgtagtgttgaaaggggaaactccttttaatgattgcatatataaacgaacttcttgagttgaatagtttctcattatgatctgtttaaacagtatggtgcagaagaacagatgaacttgttgatggcccaaacgcatcatatattaccccggcagccttagataggtgggaaaataggctagaagatgttttcaatgggcggccatttgacatgctcgatggtgctttgtccgatacagtttctaactttccagttgatattcaggttagtctaccaattctatggtctttatatttgttcaatttgcgtttgatgtcacttttgctgagggcttttctaatagcttacttcagcctagcggaaatgtttgtagttgaatctctagttctgtctcctatatctgtttctctcgtcctagatactacacatacttcatttctgttttaacattttattcgtcttttggtgttgttttgtatgtgaatcatatatttggaacagaatcattattagttcacatgatttcatttgctttcttcaatagcgtaattgtctaaccttccaatatatgttgcagccattcagagatatgattgaaggaatgcgtatggacttgagaaaatcgagatacaaaaacttcgacgaactatacctttattgttattatgttgctggtacggttgggttgatgagtgttccaattatggcgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctca

SEQ ID NO: 64 - DNA, 1347 bp, Artificial Sequence
  CaMV35S
ctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacg

SEQ ID NO: 65 - DNA, 4224 bp, Artificial Sequence
  atCas9
atggatccccgggatcatctacttctgaagactcagactcagactaagcaggtgacgaacgtcaccaatcccaattcgatctacatcgataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttga

SEQ ID NO: 66 - DNA, 771 bp, Artificial Sequence
  OCS terminator
gtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatct

SEQ ID NO: 67 - DNA, 387 bp, Artificial Sequence
  U6-26 promoter
ctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 68 - DNA, 104 bp, Artificial Sequence
  Ps#1 gRNA
ggaatgtctgttgccttgttagttttagagctagaaatagcaagttaaaataaggctagtccgttatcaacttgaaaaagtggcaccgagtcggtgcttttttt

SEQ ID NO: 69 - DNA, 23 bp, Artificial Sequence
  Arabidopsis chromosome 3 - 1228483
aactgctttgaatgtccatatgg

SEQ ID NO: 70 - DNA, 23 bp, Artificial Sequence
  Arabidopsis chromosome 3 - 1222177
gctggagaaccgccgtttaacgg

SEQ ID NO: 71 - DNA, 23 bp, Artificial Sequence
  Arabidopsis chromosome 3 - 1261146
cgcttgaatgatgaccactgcgg

SEQ ID NO: 72 - DNA, 23 bp, Artificial Sequence
  Arabidopsis chromosome 3 - 1352616
atattgtttttcatattttttgg

SEQ ID NO: 73 - DNA, 23 bp, Artificial Sequence
  Arabidopsis chromosome 3 - 1352124
ccaaaaaaaaaaaatacagtcgt

SEQ ID NO: 74 - DNA, 23 bp, Artificial Sequence
  Arabidopsis chromosome 3 - 1854159
gtttccgccaccaccgcctccgg

SEQ ID NO: 75 - DNA, 23 bp, Artificial Sequence
  Arabidopsis chromosome 3 - 1843852
tctacaaagtcattgaaggttgg

SEQ ID NO: 76 - DNA, 24 bp, Artificial Sequence
  Arabidopsis chromosome 3 - 1858597
agagttgatctgtggctgtggcgg

SEQ ID NO: 77 - DNA, 23 bp, Artificial Sequence
  Arabidopsis chromosome 3 - 4684724
tgactgcaggtgagcttacacgg

SEQ ID NO: 78 - DNA, 23 bp, Artificial Sequence
  Arabidopsis chromosome 3 - 1565357
ccttggaaattttctcttcccaa

SEQ ID NO: 79 - DNA, 23 bp, Artificial Sequence
  Arabidopsis chromosome 3 - 1559196
ccccgacatttaatgatgttttt

SEQ ID NO: 80 - DNA, 23 bp, Artificial Sequence
  Arabidopsis chromosome 3 - 4639826
agaagttcagaaagtcgcccagg

(19) United States
(12) Patent Application Publication
(10) Pub. No.: US 2021/0032645 A1
(43) Pub. Date: Feb. 4, 2021

(54) TARGETED RECOMBINATION BETWEEN HOMOLOGOUS CHROMOSOMES AND USES THEREOF

(71) Applicant: YEDA RESEARCH AND DEVELOPMENT CO. LTD., Rehovot (IL)

(72) Inventors: Avraham A. LEVY, Rehovot (IL); Cathy MELAMED-BESSUDO, Rehovot (IL); Shdema FILLER-HAYUT, Rehovot (IL)

(73) Assignee: YEDA RESEARCH AND DEVELOPMENT CO. LTD., Rehovot (IL)

(21) Appl. No.: 16/476,561

(22) PCT Filed: Jan. 11, 2018
(86) PCT No.: PCT/IL2018/050040

§ 371 Date: Jul. 9, 2019

Related U.S. Application Data
(60) Provisional application No. 62/444,827, filed on Jan. 11, 2017.

(51) Int. Cl. C12N 15/82

(52) U.S. Cl. CPC C12N 15/8213

(57) ABSTRACT

Methods of targeted recombination between homologous chromosomes in the genome of a somatic plant cell are described herein, wherein the target site may be located within a region of euchromatin or a region of heterochromatin. These methods may be used to induce a somatic plant cell into using targeted recombination between homologous chromosomes leading to targeted crossover or gene conversion. Methods described utilize a preselected endogenous target site at a locus having polymorphic alleles on the homologous chromosomes. Target site loci disclosed include those within euchromatin and heterochromatin.

### CROSS-REFERENCE TO RELATED APPLICATIONS

This application is a National Phase Application of PCT International Application No. PCT/IL2018/050040, International Filing Date Jan. 11, 2018, claiming the benefit of U.S. Patent Application No. 62/444,827, filed Jan. 11, 2017 which are hereby incorporated by reference.

### SEQUENCE LISTING STATEMENT

The instant application contains a Sequence Listing which has been submitted electronically in ASCII format and is hereby incorporated by reference in its entirety. Said ASCII copy, created on Oct. 14, 2020, is named P-80701-US-SQL-14OCT20_ST25.txt and is 98,304 bytes in size.

### FIELD OF INTEREST

Methods of targeting DNA recombination between homologous chromosomes in somatic plant cells is described, wherein these plant cells may be isolated cells, a part of an isolated plant tissue, a part of a whole plant, or a whole plant, wherein the targeted sequence may correspond to euchromatin or heterochromatin.

### BACKGROUND

DNA double-strand breaks (DSBs) are one of the powerful forces that shape plant genomes. These DSBs may occur throughout the plant life cycle, in somatic or meiotic cells, spontaneously during the movement of replication forks or developmentally controlled as in the early stages of first meiosis. They also may be induced through ionizing radiation, genotoxic drugs or through the activation of endonucleases. Unrepaired DNA DSB may cause extreme types of damage including chromosome loss, leading to gamete sterility or cell death. Repair of DSBs may also be associated with insertion/deletion (indels) mutations. DSBs repair mechanisms are therefore essential for the maintenance of genome integrity. Understanding these mechanisms is critical for the ability to precisely engineer genomes, e.g. for targeted mutagenesis, gene targeting or for other types of targeted chromosomes reshuffling.

DNA DSB repair mechanisms have been widely studied in many organisms, including plants. Studies in plants have characterized the genes involved in DSB repair via non-homologous-end-joining (NHEJ) or homologous recombination (HR) and tested the outcome of DSB repair in both somatic and meiotic tissues. NHEJ has been characterized in a broad range of species and tissues (mostly somatic), using multiple DSB inducing agents including site specific meganucleases, transposon excision, and custom-designed nucleases, such as zinc-finger nucleases (ZFNs), transcription activator-like effector nucleases (TALENs), and Clustered Regulatory Interspaced Short Palindromic Repeat associated protein Cas9 (CRISPR-Cas). The emerging picture from these works suggests that NHEJ is a prominent repair pathway in somatic cells of plants. This error-prone mechanism involves indels ranging from a few base pairs (bp) to several Kbps at the DSB site and is often associated with microhomologies. In addition, CRISPR-Cas-based systems prove to be highly efficient in a broad range of plant species, including tomato.

Naturally occurring homologous recombination in somatic plant cells is very low and near null when considering somatic recombination at a specific locus. This low frequency of homologous recombination is considered to be important in maintaining stability of the large and repetitive plant genomes. Several studies that addressed the mechanism of DSB repair via HR in somatic tissues were done in Arabidopsis, using transgenic assays that tested repair mechanisms such as intrachromosomal recombination and unequal crossover. In all cases, DSB induction enhanced HR-repair rates. Recombination rates from the unequal crossover assay were much lower than for intrachromosomal recombination. Somatic DSB repair by an homologous chromosome, using an allelic sequence, was also studied in transgenic tobacco plants, using transposable element-induced breaks: HR-repair occurred upon excision of the transposon; but was not detected with an immobile element. DSB induction could also trigger HR-mediated repair using an ectopic genomic sequence template, albeit at very low frequencies.

DSB induction of HR between endogenous (non-transgenic) recombination partners was shown in maize upon excision of the Activator (Ac), or the Mutator elements. In both cases recombination occurred in cis, between repeats flanking the transposon in somatic tissues. By contrast, germinal Ac activity did not stimulate the rate of meiotic recombination between homologous chromosomes at the maize bronze locus. This result might be due to a lack of coordination between Ac excision and meiotic recombination, a preference of meiotic HR for Spo11-induced breaks, or another unknown reason. The ability to induce HR between homologous chromosomes at a specific genomic location would provide geneticists and breeders with a powerful tool for the targeted induction of crossover or gene conversion.

Thus, there is a need for methods that will provide targeted HR between homologous chromosomes for precise breeding of crops. One application of targeted HR is targeted gene conversion, namely the transfer of a gene from one chromosome to its homolog. Such methods should also take into account the plant population size that may normally be used to achieve this goal. As well, the process of repeated backcrossing to achieve isogenic lines may also drag large segments of undesirable DNA flanking the desirable gene into the progeny plant. Disclosed herein are methods of targeted recombination between homologous chromosomes that may be performed with relatively small plant populations and without retrieval of large unwanted segments of DNA.

Plant chromosomes possess both highly condensed, heterochromatin, prominent in pericentromeric regions and corresponding to meiotic recombination cold spots and largely decondensed euchromatic regions, often corresponding to distal, subtelomeric regions and to meiotic recombination hot spots. While heterochromatin is often associated with transcriptional inactivity and suppressed genetic recombination, it does still contain transcriptionally active genes. Targeted induced recombination between homologous chromosomes in regions of heterochromatin would be an advantage in plant breeding, as in the absence of such recombination, deleterious genes may not be segregated out from beneficial genes. Disclosed herein are methods of targeting DNA recombination between homologous chromosomes in a somatic plant cell, wherein targeted DSB-induced recombination was shown to occur at both euchromatic and heterochromatic target sites.

Another potential application of DSB-induced somatic HR is “targeted crossover”, i.e. the reciprocal exchange of large chromosomal segments at a precise site. Current breeding methods rely on random crossover and search for rare recombination events, in case of linked genes, that could take tens of thousands of plants to obtain, wherein the percent homologous recombination in naturally occurring, non-targeted HR at any particular site within the genome is near 0% (occurring less than 1 in every 10⁵-10⁶ natural HR events).

Disclosed herein are methods that may be used in targeted somatic HR to combine desirable traits from the parents and to segregate between undesirable genetic linkages using small plant populations.

### SUMMARY

In one aspect, described herein is a method of targeting DNA recombination between homologous chromosomes in a somatic plant cell, said method comprising the steps of:

(a) expressing a nuclease system in said plant cell, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles;
(b) analyzing progeny of said plant cell, or a plant tissue grown from said plant cell, or a plant grown from said cell or a progeny of said plant thereof, for homologous recombination between the homologous chromosomes, wherein said homologous recombination comprises crossover or gene conversion (non-crossover); and
(c) selecting a plant cell, plant tissue thereof, plant thereof, or plant progeny thereof wherein targeted homologous recombination has occurred.

In one aspect, methods disclosed herein produce a plant comprising a combination of beneficial traits or qualities, the method comprising targeted DNA recombination between homologous chromosomes in a hybrid somatic plant cell, said method comprising the steps of:

(a) expressing a nuclease system in said plant cell, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles;
(b) analyzing progeny of said plant cell, or a plant tissue grown from said plant cell, or a plant grown from said cell or a progeny of said plant thereof, for homologous recombination between the homologous chromosomes, wherein said homologous recombination comprises crossover or gene conversion (non-crossover);
(c) selecting a plant cell, plant tissue thereof, plant thereof, or plant progeny thereof wherein targeted homologous recombination has occurred;
(d) propagating said plant cell, or plant tissue thereof, or plant thereof, or plant progeny thereof to produce a plant comprising said targeted homologous recombination, wherein said plant comprises a combination of beneficial qualities or traits not present in either parent plant from which the hybrid somatic cell originated.

In one aspect, disclosed herein is a method of producing a progeny plant comprising a combination of beneficial traits or qualities, wherein said combination is not present in either parent plant, said method comprising:

selecting parent plants, wherein each of said parents comprises at least one beneficial trait, wherein said beneficial traits are not identical and wherein said parents are polymorphic for one said at least beneficial trait;
crossing said parent plants to creates a hybrid plant;
collecting somatic cells from the hybrid plant;
expressing a nuclease system in said somatic cells, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles, wherein homologous crossover or gene conversion (non-crossover) at said targeted preselected endogenous target site leads to an exchange of DNA expressing or regulating the expression of at least one of said beneficial traits or qualities;
analyzing progeny of said plant cells, or a plant tissue grown from said plant cells, or a plant grown from said cells or a progeny of said plant thereof, for said crossover or gene conversion (non-crossover) event wherein said combination of traits is expressed;
selecting a plant cell, plant tissue thereof, plant thereof, or plant progeny thereof wherein the combination of traits is expressed; and
propagating said plant cell, plant tissue thereof, plant thereof, to produce a progeny plant that comprise said combination of beneficial traits or qualities.

In a related aspect, a nuclease system comprises a zinc finger nuclease (ZFN) system, a transcription activator-like effector nuclease (TALEN) system, or a clustered regularly interspaced short palindromic repeats (CRISPR)/CRISPR associated proteins (Cas) system.

In another aspect, a nuclease system comprises a zinc finger nuclease (ZFN) comprising a zinc-finger DNA binding domain and a DNA nuclease cleavage domain, wherein said zinc-finger DNA binding domain binds within said preselected endogenous target site, thereby targeting the DNA nuclease cleavage domain to cleave the DNA within said preselected endogenous target site. In another aspect, a nuclease system comprises a transcription activator-like effector nuclease (TALEN) system comprising a TAL effector DNA binding domain and a DNA cleavage domain, wherein said TAL effector DNA binding domain binds within said preselected endogenous target site, thereby targeting the DNA cleavage domain to cleave the DNA within said preselected endogenous target site. In another aspect, a nuclease system comprises a CRISPR/Cas nuclease system comprising a CRISPR-associated endonuclease and a gRNA molecule, wherein said gRNA molecule binds within said preselected endogenous target site thereby guiding said CRISPR-associated endonuclease to cleave the DNA within said preselected endogenous target site. In another aspect, a CRISPR-associated endonuclease (Cas nuclease) is selected from the group comprising Cas1, Cas1B, Cas2, Cas3, Cas4, Cas5, Cash, Cas7, Cas8, Cas9, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, C2c1, CasX, NgAgo, Csf1, Csf2, Csf3, and Csf4, homologs thereof, or modified versions thereof.

In a related aspect, a somatic plant cell originates from an existing hybrid or heterozygous plant cell having polymorphic alleles at said preselected site. In another aspect, an existing hybrid or heterozygous plant cell originates from a wild-type plant.

In a related aspect, a method disclosed herein produces a somatic plant cell comprising a targeted homologous recombination within the preselected endogenous target site, or a plant tissue comprising said somatic plant cell, or a plant comprising said somatic plant cell or a progeny plant thereof, or fruit derived from a plant comprising said somatic plant cell or progeny plant thereof, or seeds derived from a plant comprising said somatic plant cell or progeny plant thereof, or any combination thereof, having a combination of parental traits, said combination not present in either parent. In another aspect, a parental trait comprises increased drought resistance, increased resistance to pests, increased resistance to pathogens, improved nutrient content, or improved growth parameters, or any other trait of benefit to the plant cell, plant tissue, plant, fruit, or seed.

In a related aspect, a somatic plant cell originates from a cell from the progeny of crossing two plants, wherein said parental plant cells each comprise a polymorphic allele compared with said mate at said preselected site.

In a related aspect, a method disclosed herein produces a somatic plant cell comprising a targeted homologous recombination within the preselected endogenous target site, or a plant tissue comprising said somatic plant cell, or a plant comprising said somatic plant cell or a progeny plant thereof, or fruit derived from a plant comprising said somatic plant cell or progeny plant thereof, or seeds derived from a plant comprising said somatic plant cell or progeny plant thereof, or any combination thereof, having a resultant combination of parental traits said combination not present in either parent. In another aspect, the parental traits recombined through said targeted homologous recombination comprise increased drought resistance, increased resistance to pests, increased resistance to pathogens, improved nutrient content, or improved growth parameters, or any other trait of benefit to the plant cell, plant tissue, plant, fruit, or seed.

In a related aspect, one of said parent somatic plant cells comprises said nuclease system, and wherein the DNA cleaving activity of said nuclease system is targeted to the polymorphic allele present in the other parent plant cell that does not comprise said nuclease system.

In another related aspect, one of said parent somatic plant cells comprises a Cas nuclease and the other of said parent somatic plant cells comprises a gRNA molecule, wherein said gRNA molecule binds within said preselected endogenous target site thereby guiding said Cas nuclease to cleave the DNA within said preselected endogenous target site.

In another related aspect, a somatic plant cell comprises a cell from a plant progeny of a cross between two polymorphic parental lines, which creates a hybrid plant, wherein said parental plant lines each comprise a polymorphic allele at said preselected endogenous target site, and wherein only one of the parental lines comprises said nuclease system.

In another related aspect, a method disclosed herein produces a somatic plant cell comprising a targeted homologous recombination within the preselected endogenous target site, or a plant tissue comprising said somatic plant cell, or a plant comprising said somatic plant cell or a progeny plant thereof, or fruit derived from a plant comprising said somatic plant cell or progeny plant thereof, or seeds derived from a plant comprising said somatic plant cell or progeny plant thereof, or any combination thereof, having a combination of parental traits said combination not present in either parent. In another aspect, the parental traits comprise increased drought resistance, increased resistance to pests, increased resistance to pathogens, improved nutrient content, or improved growth parameters, or any other trait of benefit to the plant cell, plant tissue, plant, fruit, or seed.

In another related aspect, a nuclease system comprises a Cas nuclease and a gRNA molecule, wherein said gRNA molecule binds within said preselected endogenous target site thereby guiding said Cas nuclease to cleave the DNA within said preselected endogenous target site, and wherein the DNA cleaving activity of said nuclease system occurs solely on the heterologous allele present in wild-type parent plant cell.

In another related aspect, a somatic plant cell is comprised within a plant tissue or a whole plant. In another related aspect, a somatic plant cell comprises a protoplast. In another related aspect, a somatic plant cell comprises a crop plant cell.

In another related aspect, a preselected endogenous target site comprises DNA comprising a gene, a part of a gene, or a regulatory upstream or downstream sequence of a gene, or any combination thereof, and wherein expression or lack thereof of said gene affects growth, drought resistance, resistance to pests, resistance to pathogens, or nutrient content, or any other trait of benefit to the plant cell, plant tissue, plant, fruit, or seed, or any combination thereof. In another related aspect, the preselected endogenous target site comprises a region of euchromatin or heterochromatin.

In another related aspect, the expression comprises constitutive induction of expression, inducible induction of expression, tissue-specific induction of expression, or condition-specific induction of expression, or any combination thereof.

In a related aspect, analyzing said plant comprises analyzing a portion of said plant or a progeny thereof comprising a leaf, a stem, a bud, a fruit, a seed.

In a related aspect, a step of selecting progeny comprises F₁, F₂, or F₃ generations, or any subsequent generation, or backcrosses for 1 to 3 generations, or any subsequent backcross generation.

In a related aspect, a method disclosed herein produces a somatic plant cell comprising said targeted homologous recombination at said preselected endogenous target site, or a plant tissue comprising said targeted homologous recombination at the preselected endogenous target site, or a plant comprising said targeted homologous recombination at the preselected endogenous target site or a progeny plant thereof, or fruit derived from a plant comprising targeted homologous recombination at the preselected endogenous target site or progeny plant thereof, or seeds derived from a plant comprising said targeted homologous recombination at the preselected endogenous target site or progeny plant thereof, or any combination thereof, said cell, tissue, plant or progeny thereof, fruit, or seed comprising genes for increased drought resistance, increased resistance to pests, increased resistance to pathogens, improved nutrient content, improved growth parameters, or any other trait of benefit to the plant cell, plant tissue, plant or progeny thereof, fruit, or seed, or any combination thereof as compared to a control plant cell, plant or progeny thereof, fruit, or seed. In a related aspect, the preselected endogenous target site comprises a region of euchromatin or heterochromatin.

In another aspect, disclosed herein is a plant comprising a combination of beneficial traits or qualities produced by a method comprising targeted DNA recombination between homologous chromosomes in a hybrid somatic plant cell, said method comprising the steps of: (a) expressing a nuclease system in said plant cell, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles;

(b) analyzing progeny of said plant cell, or a plant tissue grown from said plant cell, or a plant grown from said cell or a progeny of said plant thereof, for homologous recombination between the homologous chromosomes, wherein said homologous recombination comprises crossover or gene conversion (non-crossover);
(c) selecting a plant cell, plant tissue thereof, plant thereof, or plant progeny thereof wherein targeted homologous recombination has occurred;
(d) propagating said plant cell, or plant tissue thereof, or plant thereof, or plant progeny thereof to produce a plant comprising said targeted homologous recombination, wherein said plant comprises a combination of beneficial qualities or traits not present in either parent plant from which the hybrid somatic cell originated. In a related aspect, the preselected endogenous target site comprises a region of euchromatin or heterochromatin.

### BRIEF DESCRIPTION OF THE DRAWINGS

The following drawings form part of the present specification and are included to further demonstrate certain embodiments of the present disclosure, the methods described herein may be better understood by reference to one or more of these drawings in combination with the detailed description of specific embodiments presented herein.

FIG. 1 presents a schematic of double strand break (DSB) repair, which may occur by non-homologous end joining (NHEJ) or homologous recombination (HR).

FIG. 2 presents a schematic embodiment of repair of a targeted DSB by Homologous Recombination (HR).

FIG. 3 presents a schematic flow chart diagram comprising some embodiments of inducing recombination between homologous chromosomes. Induction of DNA double-strand breaks are shown as yellow lightening.

FIGS. 4A-E present the tomato fruit color assay and molecular analysis for the outcomes of DNA double-strand break (DSB) repair events. FIG. 4A: The cross of yellow flesh^{e3756} 35 S:Cas9 and bicolor^{cc383} u6-26:Ps #1-sgRNA is expected to give F₁ plants with a pale Bicolor fruit phenotype. F₁ plants expressing both Cas9 and gRNA were selected. The gRNA was designed for targeted DSB induction (shown as black lightning) in both alleles between the yellow flesh^{e3756} and bicolor^{cc383} mutations (*). In case of non-homologous end-joining (NHEJ) repair of the bicolor^{cc383} allele, fruit color was expected to be yellow following error-prone repair leaving indel (insertion/deletion) footprints (blue line). In the cases of non-crossover or crossover, fruit color was expected to be red or bicolor with red spots in case of late event. FIG. 4B: Fruit phenotype distribution in F₁ plants and control: Bicolor fruits are shown as orange boxes; Yellow fruits as yellow boxes; Fruits with red sectors (putative somatic HR) are shown as red-cross-hatched boxes. Each bar represents a fruit population derived from F₁ plants originating from crosses between independent transgenic lines of Cas9 and a given u6-26:Ps #1-sgRNA line. The number of fruits analyzed in each cross is shown on the bar in black. FIG. 4C: Sequences of the NHEJ DSB repair footprints are shown on the right side and their relative frequency is shown in the pie chart. The CRISPR-Cas target sequence from the PSY1 is shown on the top (SEQ ID NO. 81: GAATGTCTGTTGCCTTGTTATGGGTTGTTTCTCC). The DSB location is shown as a black lightning; the PSY1 start codon is shown in red and the PAM-protospacer adjacent motif is shown in blue. The sequences of the NHEJ DSB repair footprints shown in FIG. 4C are derived from SEQ ID NO. 81, wherein the sequences having greater than or equal to 10 nucleotides are identified as SEQ ID NO: 82: TTGTTATGGG, SEQ ID NO: 83: GCCTTGTTATG, SEQ ID NO: 84: TGTCTGTTGCCTTG, SEQ ID NO: 85: TCTGTTGCCTTG, SEQ ID NO: 86: CTGTTGCCTTG, SEQ ID NO: 87: TGTTGCCTTG. SEQ ID NO: 88: CCTTGTTATGGGTTGTTTCT, and SEQ ID NO: 89: GTTGCCTTGTTATGG. The top pie represents an average of illumina Hiseq reads from 22 different F₁ plants of the cross of yellow flesh e³⁷⁵⁶ 35 S:Cas9×bicolor^{cc383} u6-26:Ps #1-sgRNA. In this cross 88% of the sequences deviate from the WT sequence. The low pie represents an average of ilummina Hiseq reads from 2 plants of control F₁ population (yellow flesh e³⁷⁵⁶×bicolor^{cc383} F₁ plants with no CRISPR-Cas components). The orange CTTG deletion is a preferential NHEJ footprint. FIG. 4D: Inverse PCR scheme for identification of recombinant DNA fragments. (1) DNA from separate leaves samples was first digested with ApaI(A) and HindIII(H) and then blunted. (2) Each sample was self-ligated. (3) Each sample was amplified by two different primer sets (in green and yellow). Blue—Bicolor allele; red—Yellow flesh allele; Dashed blue line—Bicolor deletion, *—Yellow flesh mutation, lightning—DSB site. FIG. 4E. Ratio of parental (P) versus recombinant (R) types (as obtained from panel C) in individual plants. Plants 1-15—F₁ plants of the cross of yellow flesh e³⁷⁵⁶ 35S:Cas9×bicolor^{cc383} u6-26:Ps #1-sgRNA; Plant 16—synthetic crossover control; Plants17-18-Yellow flesh×Bicolor (Cas9-) F₁ plants.

FIG. 5 presents NHEJ repair in somatic cells. NHEJ footprints distribution in individual F₁ plants and in control plants (yellow flesh e³⁷⁵⁶×bicolor^{cc383}) obtained by sequencing of PCR products amplified around the CRISPR-Cas9 induced DSB (lightning) with primers shown as red arrows. Each pie represents the total ilummina Hiseq reads for single plant (250,000-850,000 reads per plant). The nucleotide sequences are as described in FIG. 4C, wherein the CRISPR-Cas target sequence from the PSY1 is shown on the top-right (SEQ ID NO: 81). The sequences to the right of the pie charts represent the sequences of the NHEJ DSB repair footprints, as shown in FIG. 4C, and are derived from SEQ ID NO: 81, wherein the sequences having greater than or equal to 10 nucleotides are identified as SEQ ID NO: 82, SEQ ID NO: 83, SEQ ID NO: 84, SEQ ID NO: 85, SEQ ID NO: 86, SEQ ID NO: 87. SEQ ID NO: 88, and SEQ ID NO: 89.

FIGS. 6A-6B presents the tomato SNPs assay for the analysis of germinal DNA double-strands break (DSB) repair events. FIG. 6A. An homozygote M82 CRISPR mutant (+A,+A) expressing 35S:Cas9 and u6-26:Ps #2-sgRNA was crossed with S. pimpinellifolium^{LA1578}. The F₁ is expected to give red fruits without DNA DSB and yellow fruit in case that the break was repaired by NHEJ, non-crossover or crossover. The SNPs pattern allows differentiating between repair mechanisms. Triangles are for SNPs; lightning mark the DSB site; blue line is for NHEJ indels. FIG. 6B. Analysis of DNA DSB flanking markers in F₂ and F₃ plants. Red—homozygote for S. pimpinellifolium^{LA1578} SNPs; yellow-homozygote for M82 SNPs (including the +A CRISPR-Cas9 mutant); orange—heterozygote; empty cells are for missing data; lightning—DSB site.

FIG. 7 presents the tomato SNPs assay for allele-specific DNA DSB repair. DNA was extracted from 4 leaves of M82 35S:Cas9 u6-26:Ps #2-sgRNA psy1^{+A}/psy1^{+A}, S. pimpinellifolium^{LA1578} and 5 plants of their F₁ inbred. illumina sequencing was preformed and each pie represent a summary of 600,000-900,000 reads per plant. The sequences to the left of the pie charts represent the sequences of the allele-specific DNA DSB repair sequences, wherein the sequence greater than or equal to 10 nucleotides is identified as SEQ ID NO: 90: CGTATATAATGCTG.

FIG. 8 presents a schematic map of fruit color phenotypes throughout development and sequencing of DNA DSB repair footprints from fruit pericarp tissues using illumina sequencing. An example is shown for plant #1, which is a F₁ plant of M82 35S:Cas9 u6-26:Ps #2-sgRNA psy1^{+A}/psy1^{+A}×S. pimpinellifolium^{LA1578}. Fruit color phenotype was variable from red through red with small or big yellow sectors to yellow. Each pie was built out of 15,000-50,000 illumina sequencing reads per fruit.

FIGS. 9A and 9B present quantification of allele-dependent repair. FIG. 9A. Two plant populations were grown, both in the M82 background: one homozygote for PSY1/PSY1 and the other heterozygote for the PSY1/psy^{+A} genotype. Progeny of these plants could give a +A SNP at the site of the break (lightning) or any other mutation (*). DNA was extracted from leaves of 4 weeks old plants of both populations and sequenced by illumina. In the PSY1/PSY1 plants, both alleles can be targeted, while in the PSY1/psy^{+A} plants, only the WT PSY1 allele is targeted. FIG. 9B. The percent of +A mutation per WT allele in PSY1/PSY1 plants served as expected value for allele-independent+A mutation. It was calculated by the following equations: Expected=(%(+A reads)_{T=4 weeks (wt,wt)})/2. To estimate the observed occurrence of the +A mutation when the second allele has a +A mutation (in M82-WT PSY1/M82 psy1^{+A} heterozygote plants) as shown in FIG. 9A, the equation used was: Observed=%(+A reads)_{T=4 weeks, (wt,+A)}−50%. The bars correspond to the standard error for 22 PSY1/PSY1 plants and 14 PSY1/psy^{+A} plants. The difference between the means was significant (p value (Wilcoxon rank sum test)=0.009).

FIG. 10 shows a DNA DSB repair event followed by fruit phenotype and pericarp specific illumina sequencing—plant #2. All details are similar to FIG. 8. This plant showed high level of psy1^{+A}. The conversion products in FIG. 6B are the progeny of this plant.

FIG. 11 presents Table 10, which tabulates the CRISPR DSB targets on Arabidopsis chromosome 3.

FIGS. 12A-12C show the Arabidopsis system for somatic DNA DSB induction at recombination hot and cold spots. (FIG. 12A) Twelve meiotic recombination targets in regions considered as hot or cold spots between GFP and RFP seed markers. Hot (in red) and Cold (in blue) targets had features of euchromatin or heterochromatin characteristic of hot spots or cold spots of recombination, respectively. The coordinates of the targets and their distribution on chromosome 3 between the GFP and RFP markers are shown. FIG. 12B shows the experimental scheme: twelve homozygote Columbia tester lines expressing 35S×2: Hygromycin, u6-26:gRNA cassette, each encoding for gRNA targeting specific hot/cold sequence were crossed with WT Columbia lines expressing nos:nptII:nos Ubi:spCas9. Recombination rates were calculated based on F₂ self-fertilized seeds that were used to calculate the crossover rate between the GFP and RFP markers-left side (Results shown in FIG. 12C). In addition, F1 plants were crossed with wild type Landsberg plants and DNA from somatic tissues were extracted for determining the somatic rate and the mechanism of DNA DSB repair around the DSB by PacBio sequencing (Results shown in FIGS. 13A-13Q). (FIG. 12C) Crossover rate in CentiMorgan (Y axis) between the GFP and RFP markers following CRISPR-Cas9 DSB induction at targets shown on the X-axis with coordinate number for hot (red) or cold (blue) sites. Controls in the absence of DSB induction are shown in black. The large red diamond represents average crossover rate for each population.

FIGS. 13A-13Q present the molecular analysis of DSB repair at Hot Target—chr3:1854159 using Pacbio sequencing. DNA was purified from young buds (at pre-meiosis stage), stems and upper leaves tissue of each plant of backcrossed populations of Columbia tester×Landsberg. 5 kb fragments flanking the DNA DSB site were amplified by PCR and sequenced using PacBio. Row reads were clustered to consensus sequences using PacBio Long Amplicon Analysis and then aligned to the Arabidopsis genome using Burrows-Wheeler Aligner (BWA) software, and plotted. Red stripes represent Columbia (Col) single nucleotide polymorphisms (SNPs) and Blue stripes represent Landsberg (Ler) SNPs. The DSB site at target #1854179 on Chromosome 3 is shown as the dashed line. The Yellow line indicates NHEJ footprints at the DSB site. Green lines represent sequences that do not belong to any of the parents. For each plant (FIGS. 13A-13Q, wherein each box is a different plant and FIGS. 130-13Q are control plants), the extracted DNA was barcoded (separate squares with indicated barcode) hundreds or thousands of single molecules were sequenced and clustered according to sequence (including SNPs patterns). This method permits distinguishing between the parental origin of each molecule. In some plants (e.g. barcode 89 on top left square) there was no evidence of any change and the parental alleles were more or less in equal proportion. In other plants (e.g. Barcode 90—second plant top, starting from the left side), there was evidence for crossover flanking the break as suggested from the transition of red to blue SNPs in 10-12% of the molecules. Three control plants of F₁ Ler×Col tester were also sequenced and analyzed in the same way, and did not show any crossover or gene conversion event.

### DETAILED DESCRIPTION

In the following detailed description, numerous specific details are set forth in order to provide a thorough understanding of the methods presented herein. However, it will be understood by those skilled in the art that these methods of targeting DNA recombination between homologous chromosomes in a somatic plant cell or tissue or plant thereof may be practiced without these specific details. In other instances, well-known methods, procedures, and components have not been described in detail so as not to obscure the methods and resultant plant cells and plants thereof comprising DNA comprising the targeted homologous recombination, as disclosed herein.

In one embodiment, disclosed herein is a method of targeting DNA recombination between homologous chromosomes in somatic plant cells, said method comprising the steps of: (a) expressing a nuclease system in said plant cell, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles; (b) analyzing progeny of said plant cell, or a plant tissue grown from said plant cell, or a plant grown from said cell or a progeny of said plant thereof, for homologous recombination between the homologous chromosomes, wherein said homologous recombination comprises crossover or gene conversion (non-crossover); and (c) selecting a plant cell, plant tissue thereof, plant thereof, or plant progeny thereof wherein homologous recombination has occurred.

In one embodiment, methods disclosed herein produce a plant comprising a combination of beneficial traits or qualities, said method comprising targeted DNA recombination between homologous chromosomes in a hybrid somatic plant cell comprising polymorphic alleles on said homologous chromosomes, said method comprising the steps of: (a) expressing a nuclease system in said plant cell, wherein said expressed nuclease system is targeted to a preselected endogenous target site comprising the polymorphic alleles, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles; (b) analyzing progeny of said plant cell, or a plant tissue grown from said plant cell, or a plant grown from said cell or a progeny of said plant thereof, for homologous recombination between the homologous chromosomes, wherein said homologous recombination comprises crossover or gene conversion (non-crossover); (c) selecting a plant cell, plant tissue thereof, plant thereof, or plant progeny thereof wherein targeted homologous recombination has occurred; (d) propagating said plant cell, or plant tissue thereof, or plant thereof, or plant progeny thereof to produce a plant comprising said targeted homologous recombination, wherein said plant comprises a combination of beneficial qualities or traits not present in either parent plant from which the hybrid somatic cell originated.

In one embodiment, a method disclosed herein comprises producing a progeny plant comprising a combination of beneficial traits or qualities, wherein said combination is not present in either parent plant of the progeny, said method comprising: (a) selecting parent plants, wherein each of said parents comprises at least one beneficial trait, wherein said at least one beneficial traits are not identical and wherein said parents are polymorphic for one of said at least one beneficial trait; (b) crossing said parent plants to creates a hybrid plant; (c) collecting somatic cells from the hybrid plant; (d) expressing a nuclease system in said somatic cells, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles, wherein homologous crossover or gene conversion (non-crossover) at said targeted preselected endogenous target site leads to an exchange of DNA expressing or regulating the expression of at least one of said beneficial traits or qualities; (e) analyzing progeny of said plant cells, or a plant tissue grown from said plant cells, or a plant grown from said cells or a progeny of said plant thereof, for said crossover or gene conversion (non-crossover) event wherein said combination of traits is expressed; (f) selecting a plant cell, plant tissue thereof, plant thereof, or plant progeny thereof wherein the combination of traits is expressed; and (g) propagating said plant cell, plant tissue thereof, plant thereof, to produce a progeny plant that comprises said combination of beneficial traits or qualities.

In one embodiment, a plant cell is an isolated plant cell. In another embodiment, a plant cell is comprised within a plant tissue. In another embodiment, a plant cell is comprised within a whole plant. One of ordinary skill in the art would appreciate that the use throughout of the term “plant cell” comprises in different embodiments an isolated plant cell, a plant cell comprised within a plant tissue, or a plant cell comprised within a whole plant, or a combination thereof.

In some embodiments, the origin of a plant cell described herein is from a wild-type plant. In some embodiments, the origin of a plant cell is from a cultivated plant that has been selected for desirable characteristics that can be maintained by propagation. Cultivated plants may also be known as cultivars, though some cultivars have arisen in the wild.

One of ordinary skill in the art would appreciate that methods of targeting DNA recombination between homologous chromosomes is somatic plant cells, as described herein, may encompass uses for precise breeding of crops.

In some embodiments, methods of targeting DNA recombination between homologous chromosomes result in deletion of a specific allele or portion thereof. In some embodiments, an allele encodes a polypeptide whose expression provides a trait or quality beneficial to a plant or a plant product, for example a fruit or a flower. In some embodiments, an allele encodes a polypeptide whose expression enhances a beneficial trait or quality in a plant. In some embodiments, methods of targeting DNA recombination between homologous chromosomes result in addition of a specific allele or portion thereof. In some embodiments, methods of targeting DNA recombination between homologous chromosomes result in introduction of a DNA mutation within an allele. In some embodiments, methods of targeting DNA recombination between homologous chromosomes result in substitution of one allele for another allele. In some embodiments, methods of targeting DNA recombination between homologous chromosomes result in to deletion of a regulatory up-stream gene sequence of an allele. In some embodiments, methods of targeting DNA recombination between homologous chromosomes result in deletion of down-stream gene sequence of an allele. In some embodiments, methods of targeting DNA recombination between homologous chromosomes result in addition of a regulatory up-stream gene sequence of an allele. In some embodiments, methods of targeting DNA recombination between homologous chromosomes result in down-stream gene sequence of an allele. In some embodiments, methods of targeting DNA recombination between homologous chromosomes result in mutation of a regulatory up-stream gene sequence. In some embodiments, methods of targeting DNA recombination between homologous chromosomes result in down-stream gene sequence. In some embodiments, methods of targeting DNA recombination between homologous chromosomes result in deletion of a specific allele or portion thereof or addition of a specific allele or portion thereof or introduction of a DNA mutation within an allele or substitution of one allele for another allele or deletion of a regulatory up-stream gene sequence of an allele or deletion of down-stream gene sequence of an allele or addition of a regulatory up-stream gene sequence of an allele or regulatory down-stream gene sequence of an allele or mutation of a regulatory up-stream gene sequence or of a regulatory down-stream gene sequence, or any combination thereof of an allele.

In some embodiments, methods of targeted DNA recombination between homologous chromosomes result in allele replacement. In one embodiment, allele replacement comprises replacing a wild-type gene with a mutant allele at the endogenous locus. In another embodiment, allele replacement comprises replacing a mutant allele with a wild-type allele at the endogenous locus. In another embodiment, allele replacement comprises replacing a mutant allele with a different mutant allele at the endogenous locus. In some embodiment, allele replacement results in expression of a beneficial trait or quality for the plant cell, tissue thereof, plant thereof or progeny thereof. An advantage of methods disclosed herein for allele replacement is that there is no need to develop exogenous nucleic acid sequence comprising the replacement allele, for example vectors comprising the replacement alleles. The exchange of allelic material is between homologous chromosomes in a cell, wherein the chromosomes comprise polymorphic alleles.

In some embodiments, methods of targeted DNA recombination between homologous chromosomes result in single nucleotide polymorphism (SNP) replacement. In one embodiment, a SNP replacement comprises creating a missense mutation in a gene. In another embodiment, a SNP replacement comprises placing a missense mutation with the wild-type nucleotide. In another embodiment, a SNP replacement comprising creating a missense mutation in a gene that enhances the function of the encoded polypeptide. In another embodiment, a SNP replacement comprising creating a missense mutation in a gene that decreases the function of the encoded polypeptide. In another embodiment, a SNP replacement comprising creating a missense mutation in a gene that enhances expression of the encoded polypeptide. In another embodiment, a SNP replacement comprising creating a missense mutation in a gene that decreases the expression of the encoded polypeptide. In some embodiment, SNP replacement results in expression of a beneficial trait or quality for the plant cell, tissue thereof, plant thereof or progeny thereof. An advantage of methods disclosed herein for SNP replacement is that there is no need to develop exogenous nucleic acid sequence comprising the replacement SNP, for example vectors comprising the replacement SNP. The exchange of nucleic acid sequence comprising a SNP is between homologous chromosomes in a cell, wherein the chromosomes comprise polymorphic alleles.

In some embodiments, methods of targeting DNA recombination between homologous chromosomes results in the transfer of a single locus from one chromosome to its homolog via homologous recombination (HR), wherein a new desired combination of traits is generated in a progeny plant cell. In some embodiments, methods of targeting DNA recombination between homologous chromosomes results in the transfer of a single locus from one chromosome to its homolog via homologous recombination (HR), wherein a new desired combination of traits is generated in a progeny plant tissue. In some embodiments, methods of targeting DNA recombination between homologous chromosomes results in the transfer of a single locus from one chromosome to its homolog via homologous recombination (HR), wherein a new desired combination of traits is generated in a progeny plant. In some embodiments, transfer of a single locus comprises reshuffling of chromosomal fragments from one chromosome to its homolog via homologous recombination (HR), wherein a new desired combination of traits is generated in a progeny plant cell. In some embodiments, transfer of a single locus comprises reshuffling of chromosomal fragments from one chromosome to its homolog via homologous recombination (HR), wherein a new desired combination of traits is generated in a progeny plant tissue. In some embodiments, transfer of a single locus comprises reshuffling of chromosomal fragments from one chromosome to its homolog via homologous recombination (HR), wherein a new desired combination of traits is generated in a progeny plant. In some embodiments, the combination of traits is not present in either parent.

In some embodiments, a locus comprises an allele. In some embodiments, a locus comprises a part of an allele. In some embodiments, a locus comprises upstream sequence of an allele. In some embodiments, a locus comprises downstream sequence of an allele. In some embodiments, a locus comprises a single SNP within an allele. In some embodiments, a locus comprises multiple SNPs within an allele. In some embodiments, a locus comprises a contiguous nucleic acid sequence comprising an allele, upstream sequence of the allele, downstream sequence of the allele, a regulatory sequence of the allele, or a SNP within the allele, or any combination thereof.

Generation of a new desired trait or combination of traits is difficult to obtain via natural recombination, for example during cultivation of plants, wherein recombination is not targeted to a specific locus, and where recombination at a specific locus occurs less than 10⁵-10⁶-times/per natural recombination event.

In one embodiment, a single locus comprises a gene. In one embodiment, a single locus comprises an allele. In one embodiment, a single locus comprises a portion of a gene. In one embodiment, a single locus comprises a portion of an allele. In one embodiment, a single locus comprises a gene promoter. In one embodiment, a single locus comprises a gene exon. In one embodiment, a single locus comprises at least one exon of a gene. In one embodiment, a single locus comprises at least two exons of a gene. In one embodiment, a single locus comprises at least three exons of a gene. In one embodiment, a single locus comprises a gene intron. In one embodiment, a single locus comprises at least one intron of a gene. In one embodiment, a single locus comprises at least two introns of a gene. In one embodiment, a single locus comprises at least three introns of a gene. In one embodiment, a single locus comprises at least one exon and one intron of a gene. In one embodiment, a single locus comprises any combination of exon(s) and intron(s) of a gene. In one embodiment, a single locus comprises a sequence of DNA encoding a small RNA. In one embodiment, a single locus comprises a sequence of DNA encoding a microRNA. In one embodiment, a single locus comprises a sequence of DNA encoding a tRNA. In one embodiment, a single locus comprises a sequence of DNA encoding a gene regulatory sequence or regulatory sequences.

In one embodiment, methods of targeted recombination between homologous chromosomes result in deletion of a specific allele or portion thereof. In another embodiment, methods of targeted recombination between homologous chromosomes result in addition of a specific allele or portion thereof. In another embodiment, methods of targeted recombination between homologous chromosomes result in introduction of a DNA mutation within an allele. In another embodiment, methods of targeted recombination between homologous chromosomes result in substitution of one allele for another allele. In another embodiment, methods of targeted recombination between homologous chromosomes result in deletion of a regulatory up-stream or down-stream gene sequence of an allele. In another embodiment, methods of targeted recombination between homologous chromosomes result in addition of a regulatory up-stream or down-stream gene sequence of an allele. In another embodiment, methods of targeted recombination between homologous chromosomes result in mutation of a regulatory up-stream or down-stream gene sequence.

In another embodiment, a mutation comprises a point mutation, a deletion mutation, a substitution mutation, or an insertion mutation, or any combination thereof. In another embodiment, methods of targeted recombination between homologous chromosomes result in a point mutation. In another embodiment, methods of targeted recombination between homologous chromosomes result in a deletion mutation. In another embodiment, methods of targeted recombination between homologous chromosomes result in a substitution mutation. In another embodiment, methods of targeted recombination between homologous chromosomes result in an insertion mutation.

In some embodiments, methods disclosed herein “knock-out” a gene, wherein a skilled artisan would appreciate that “knocking out” a gene encompasses making inoperative a gene within the plant genome. In some embodiments, a gene knock-out leads to expression of a beneficial quality or trait in a plant. In some embodiments, a gene knock-out leads to increased expression of a beneficial quality or trait in a plant. In some embodiments, a gene knock-out leads to reduced expression of a negative quality or trait in a plant. In some embodiments, a gene knock-out leads to lack of expression of a non-beneficial quality or trait in a plant. In some embodiments, the knock-out exchanges polymorphic alleles of a gene.

In some embodiments, methods disclosed herein “knock-in” a gene, wherein a skilled artisan would appreciate that “knocking in” a gene encompasses making operative a gene within the plant genome that was not previously expressed therein. In some embodiments, a gene knock-in leads to expression of a beneficial quality or trait in a plant. In some embodiments, a gene knock-in leads to increased expression of a beneficial quality or trait in a plant. In some embodiments, a gene knock-in leads to reduced expression of a negative quality or trait in a plant. In some embodiments, a gene knock-in leads to lack of expression of a non-beneficial quality or trait in a plant. In some embodiments, the knock-in exchanges polymorphic alleles of a gene.

A skilled artisan would appreciate that “homologous recombination” encompasses a mechanism of genetic recombination in which two DNA strands comprising similar nucleotide sequences exchange genetic material. Cells use homologous recombination during meiosis, where it serves to rearrange DNA to create an entirely unique set of haploid chromosomes. Somatic cells may use homologous recombination for the repair of damaged DNA, in particular for the repair of double strand breaks (DSB). In one embodiment, as described herein, homologous recombination is induced to occur between homologous chromosomes comprising polymorphic alleles in a somatic cell. The homologous recombination event can be used to alter an endogenous gene in any number of ways. In some embodiment, the homologous recombination can result in gene conversion (non-crossover). In some embodiment, the homologous recombination may lead to inactivation of an endogenous gene. In some embodiments, the homologous recombination may produce a recombinant locus, for example an allele, derived from two related genes. The newly created recombinant allele may in one embodiment have a new activity as compared to either of the genes from which it was derived. Changes in methylation patterns in DNA may lead to changes in expression of a gene or genes. In some instances this may be beneficial, while in other instances changes in methylation patterns have been shown to be involved in disease states such as cancer. In some embodiments, methods of targeted homologous recombination disclosed herein may lead to changes of methylation at the epigenetic level that is, a change in methylation pattern. In other embodiments, methods of targeted homologous recombination does not lead to changes of methylation at the epigenetic level that is, there is no change in methylation pattern.

In some embodiment, a targeted DNA recombination between homologous chromosomes in a somatic cell, wherein the target site for recombination comprises polymorphic alleles, is heritable, wherein the recombination event is transmitted to progeny. Thus, once a plant cell, plant tissue propagated from a cell, or a plant propagated from a cell is analyzed and selected as comprising a targeted homologous recombination event, progeny comprising this targeted recombination event may be generated. In one embodiment, the recombinant event is heritable through seeds via the germline of a plant propagated from a cell or tissue comprising a targeted DNA recombination as disclosed herein. In another embodiment, the recombinant event is heritable through regeneration of vegetative tissue containing the recombinant event. In another embodiment, the recombinant event is heritable through propagation of vegetative tissues containing the heritable event. Non-limiting examples of propagation of vegetative tissue comprising the recombination events disclosed herein include use of a branch comprising the recombinant event to make a tree cutting or for grafting onto a tree, and use of a callus comprising a recombinant event to regenerate a banana plant.

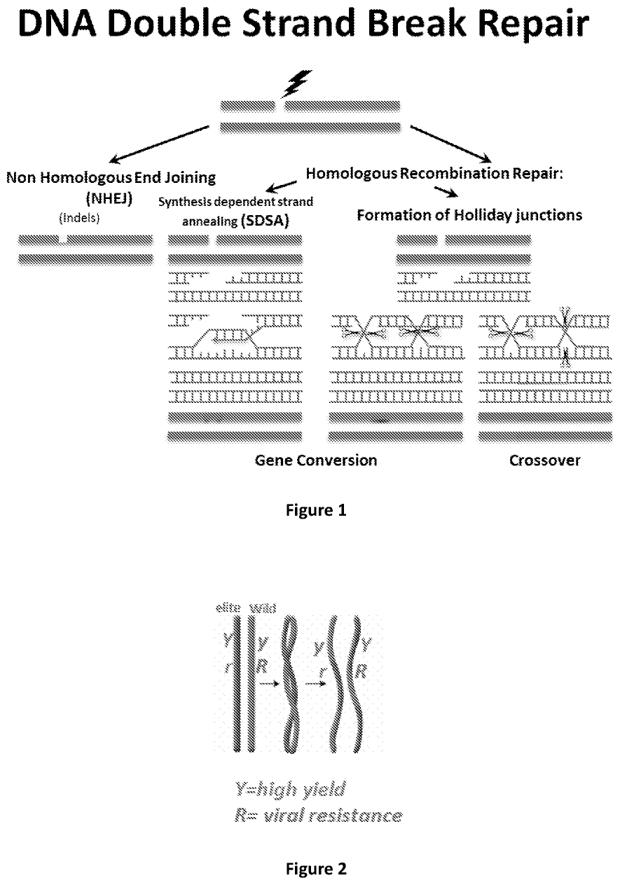

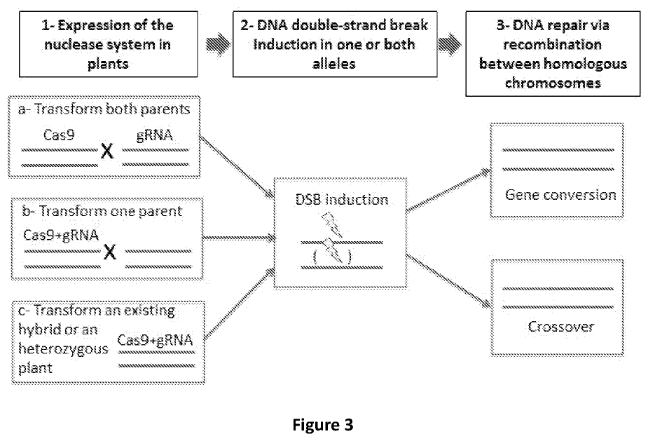

DNA DSB can serve as a powerful tool to change and control plant genomes. In plants, most of the DNA double strand breaks will be repaired by the NHEJ machinery, which usually leaves small Indels at the break site. (FIG. 1) The break may also be repaired by Homologues Recombination (HR). (FIG. 1, FIG. 2, and FIG. 3—right-hand side). In one embodiment, when HR is solved by synthesis dependent strand annealing, the result is gene conversion (transfer of a locus from one chromosome to the other chromosome; also known as a non-crossover event). In another embodiment, when HR is solved by the formation of Holliday junctions the result is a gene conversion event or a crossover event, depending how the Holliday junction has been resolved. A skilled artisan would appreciate that a homologous recombination “crossover” event between homologous chromosomes encompasses strand exchange between DNA sequences. In one embodiment, a crossover event comprises exchange between DNA sequences comprising substantially similar nucleotide composition. In another embodiment, a crossover event comprises exchange between DNA sequences of homologous chromosomes comprising polymorphic alleles, wherein the crossover event encompasses strand exchange between DNA sequences comprising a polymorphic allele. In other words, homologous recombination by crossover of homologous chromosomes comprising a polymorphic allele may result in an extended exchange of DNA sequence wherein the sequence comprises sequence comprising a different nucleotide composition. Further, homologous recombination crossover events, in another embodiment, provide for the exchange of DNA sequence flanking a DSB.

In some embodiments, methods disclosed herein of targeted homologous recombination within an endogenous target site comprise an exchange of contiguous DNA sequence wherein said contiguous DNA sequence comprises about 0.01 KB-20 KB DNA. In some embodiments, methods disclosed herein of targeted homologous recombination within an endogenous target site comprise an exchange of contiguous DNA sequence wherein said contiguous DNA sequence comprises about 0.1 KB-20 KB DNA. In some embodiments, methods disclosed herein of targeted homologous recombination within an endogenous target site comprise an exchange of contiguous DNA sequence wherein said contiguous DNA sequence comprises about 1 KB-20 KB DNA.

In some embodiments, methods of targeted homologous recombination comprise an exchange of about 1 KB-5 KB. In some embodiments, methods of targeted homologous recombination comprise an exchange of about 5 KB-10 KB. In some embodiments, methods of targeted homologous recombination comprise an exchange of about 10 KB-15 KB. In some embodiments, methods of targeted homologous recombination comprise an exchange of about 15 KB-20 KB.

In some embodiments, methods of targeted homologous recombination comprise an exchange of at least about 1 KB. In some embodiments, methods of targeted homologous recombination comprise an exchange of at least about 2 KB. In some embodiments, methods of targeted homologous recombination comprise an exchange of at least about 3 KB. In some embodiments, methods of targeted homologous recombination comprise an exchange of at least about 4 KB. In some embodiments, methods of targeted homologous recombination comprise an exchange of at least about 5 KB. In some embodiments, methods of targeted homologous recombination comprise an exchange of at least about 6 KB. In some embodiments, methods of targeted homologous recombination comprise an exchange of at least about 7 KB. In some embodiments, methods of targeted homologous recombination comprise an exchange of at least about 8 KB. In some embodiments, methods of targeted homologous recombination comprise an exchange of at least about 9 KB. In some embodiments, methods of targeted homologous recombination comprise an exchange of at least about 10 KB. In some embodiments, methods of targeted homologous recombination comprise an exchange of at least about 11 KB. In some embodiments, methods of targeted homologous recombination comprise an exchange of at least about 12 KB. In some embodiments, methods of targeted homologous recombination comprise an exchange of at least about 13 KB. In some embodiments, methods of targeted homologous recombination comprise an exchange of at least about 14 KB. In some embodiments, methods of targeted homologous recombination comprise an exchange of at least about 15 KB. In some embodiments, methods of targeted homologous recombination comprise an exchange of at least about 16 KB. In some embodiments, methods of targeted homologous recombination comprise an exchange of at least about 17 KB. In some embodiments, methods of targeted homologous recombination comprise an exchange of at least about 18 KB. In some embodiments, methods of targeted homologous recombination comprise an exchange of at least about 19 KB. In some embodiments, methods of targeted homologous recombination comprise an exchange of at least about 20 KB.

FIG. 2 schematically demonstrates how targeted break repair may be used as a precise breeding tool. The repair products of these breaks can be very useful for breeding. In one embodiment, homologous recombination is used to deliver traits from a wild-type plant variety into a known cultivar. In another embodiment, homologous recombination is used to deliver traits from one known cultivar having a specific trait into a second known cultivar lacking the specific trait. In some embodiments, DSB repair by homologous recombination “breaks” a very tight genetic linkage of 2 genes involved in important traits. For example: when a gene which involved in disease resistance (FIG. 2 “R”) is located next to a gene involved in high yield production (FIG. 2 “Y”), the naturally occurring meiotic crossover can be very low. Inducing a targeted DSB between these two genes, between Yr on the blue chromosome and yR on the red chromosome, followed by homologous crossover repair will dissociate between the two linked trait enabling to generate the new recombinant combination in a progeny with high yield and resistance to the disease. This also enables to minimize the length of chromosomal segments from wild-type varieties that may contain undesired genes.

In one embodiment, methods of targeted recombination between homologous chromosomes, as disclosed herein, differ from methods of gene targeting by homologous recombination that involve the exchange of genetic information between genomic and exogenous deoxyribonucleic acid (DNA) molecules via homologous recombination. In another embodiment, a method of targeted recombination between homologous chromosomes, as disclosed herein, does not involve or require or use an exogenous homologous fragment of DNA as a template for homologous recombination.

Methods described herein are advantageous compared with gene targeting methods known in the art that require exogenous DNA fragments as templates for exchange of genetic information between genomic and exogenous deoxyribonucleic acid (DNA) molecules via homologous recombination. Another advantage is that the resultant plant cell from which progeny plant tissue and whole plants may be generated is not transgenic. In one embodiment, a progeny plant cell, plant tissue, or whole plant produced using the methods described herein does not comprise foreign DNA from the nuclease system that can be eliminated, for example by genetic segregation or that can be provided by transient expression. The targeted homologous recombination described herein mimics the natural phenomenon of homologous recombination, but because the DSB is targeted, the recombinant DNA event is targeted to exchange, for example, advantageous traits. Another advantage of using methods of targeted recombination described herein that obtaining a plant comprising the desired event would entail screening tens of thousands of plants in order to identify a plant comprising the specific exchange of traits by natural (non-induced/non-targeted) homologous recombination. Another additional advantage over methods using exogenous DNA, is that it has been shown that exogenous DNA may change the DNA methylation pattern at the insertion site. In one embodiment, methods of targeted recombination disclosed herein change the DNA methylation pattern at the site of gene conversion. In another embodiment, methods of targeted recombination disclosed herein change the DNA methylation pattern at a crossover site. In one embodiment, methods of targeted recombination disclosed herein do not change the DNA methylation pattern at the site of gene conversion. In another embodiment, methods of targeted recombination disclosed herein do not change the DNA methylation pattern at a crossover site.

In one embodiment, methods disclosed herein are used with somatic plant cells. A skilled artisan would appreciate that a somatic plant cell encompasses any plant cell except germline cells. In another embodiment, somatic plant cells are selected from the group comprising root cells, rhizoid cells, bulb cells, stem cells, leaf cells, bud cells, seed pod cells, or fruit cells. In some embodiments, a somatic plant cell made by the methods disclosed herein may be grown under the proper conditions known in the art in order to generate a plant tissue comprising DNA comprising the targeted HR event, for example the gene conversion or crossover event. In one embodiment, a plant tissue comprises a root tissue, a rhizoid tissue, a bulb tissue, a stem tissue, a leaf tissue, a bud tissue, a tuber tissue, a tree cutting, a plant callus, a seed or a seed pod, or a fruit tissue, or any combination thereof. In another embodiment, a plant tissue grown from a plant cell made by methods disclosed herein may be used to produce a progeny plant, for example a cutting may be used to produce a tree or a part of a tree in the case of grafting.

In some embodiments, a somatic plant cell comprising a targeted DNA recombination using the methods disclosed herein, may be grown under the proper conditions known in the art in order to generate a whole plant, wherein said plant comprises the resultant targeted DNA recombination.

In another embodiment, a whole plant comprising the resultant targeted DNA recombination comprises the recombinant DNA in tissues throughout the plant. In another embodiment, the whole plant comprises the recombinant DNA in tissues in just a portion of the plant. For example, in another embodiment, the whole plant comprises the recombinant DNA in a fruit. In another embodiment, the whole plant comprises the recombinant DNA in seeds. In another embodiment, the whole plant comprises the recombinant DNA in seed pods. In another embodiment, the whole plant comprises the recombinant DNA in pollen. In another embodiment, the whole plant comprises the recombinant DNA in leaves. In another embodiment, the whole plant comprises the recombinant DNA in root tissue. In another embodiment, the whole plant comprises the recombinant DNA in rhizoid tissue. In another embodiment, the whole plant comprises the recombinant DNA in bulb tissue. In another embodiment, the whole plant comprises the recombinant DNA in stems. In another embodiment, the whole plant comprises the recombinant DNA in buds. In another embodiment, the whole plant comprises the recombinant DNA in fruits, seeds, seed pods, leaves, root tissue, rhizoid tissue, bulb tissue, stems, or buds, or any combination thereof.

In some embodiments, a somatic plant cell comprises a protoplast. A skilled artisan would appreciate that a protoplast encompasses a plant cell that has had its protective cell wall partly or totally removed, for example, by enzymatic treatment resulting in an intact biochemical competent unit of living plant that can regenerate the cell wall and further grow into a whole plant under proper growing conditions. The cell wall of a plant may also be partly or totally removed using mechanical treatments, wherein an intact biochemical competent unit of living plant is product that can regenerate the cell wall and further growth into a whole plant under proper growing conditions.

In some embodiments, methods disclosed herein making a somatic plant cell comprising DNA comprising a targeted homologous recombination event as disclosed herein, wherein said plant cell comprises a protoplast, may be used to make a plant tissue by growing the protoplast under the proper growing conditions known in the art in order to regenerate the cell wall and then growth plant tissue. In some embodiments, method disclosed herein making a somatic plant cell comprising DNA comprising a targeted homologous recombination event as disclosed herein, wherein said plant cell comprises a protoplast, may be used to make a whole plant by growing the protoplast under the proper growing conditions known in the art in order to regenerate the cell wall and then growth the whole plant.

In some embodiments, methods described herein use targeted recombination between homologous chromosomes. A skilled artisan would appreciate that the term homologous chromosomes encompasses chromosomes that contain information for the same biological features and contain the same genes at the same loci but possibly different alleles of those genes. In some embodiment, homologous chromosomes encompass chromosomes that contain information for the same biological features and contain the same genes at the same loci but have different methylation patterns for those genes, which may affect expression levels of the genes.

A skilled artisan would appreciate that the term “allele(s)” may encompass any of one or more alternative forms of a gene at a particular locus. In a diploid (or amphidiploid) cell of a plant, alleles of a given gene are located at a specific location or locus (loci plural) on a chromosome. One allele is present on each chromosome of the pair of homologous chromosomes. In one embodiment, polymorphic alleles comprise alleles which are dissimilar at corresponding chromosomal loci. The term “polymorphic alleles” may be used interchangeably with “heterologous alleles” or “heterozygous alleles” having all the same meanings and qualities.

Further, the skilled artisan would appreciate that the term “locus” (loci plural) encompasses a specific place or places or a site on a chromosome where for example a gene or genetic marker is found. In some embodiments, a locus is comprised within a region of euchromatic DNA. In some embodiments, a preselected endogenous target site comprises a region of heterochromatic DNA. In some embodiments, a preselected endogenous target site comprises a region of euchromatic DNA or heterochromatic DNA.

In some embodiments, a preselected endogenous target site comprises a locus on a chromosome where a gene or a genetic marker is found. In another embodiment, a preselected endogenous target site comprises an exon of a gene. In another embodiment, a preselected endogenous target site comprises an intron of a gene. In another embodiment, a preselected endogenous target site comprises multiple exons and introns of a gene. In another embodiment, a preselected endogenous target site comprises a region including the boundary between at least one exon and one intron. In another embodiment, a preselected endogenous target site comprises up-stream regulatory sequences. In another embodiment, a preselected endogenous target site comprises down-stream regulatory sequences. In another embodiment, a preselected endogenous target site comprises regulatory sequences located within the gene locus. In another embodiment, a preselected endogenous target site comprises up-stream sequences. In another embodiment, a preselected endogenous target site comprises down-stream sequences.

In some embodiments, a preselected endogenous target site comprises a region of euchromatic DNA. In some embodiments, a preselected endogenous target site comprises a region of heterochromatic DNA. In some embodiments, a preselected endogenous target site comprises a region of euchromatic DNA or heterochromatic DNA.

A skilled artisan would appreciate that plant chromosome possess both highly condensed, pericentromeric heterochromatin and largely decondensed euchromatic arms. Heterochromatin is often associated with transcriptional inactivity and suppressed genetic recombination. Yet, while heterochromatin may be gene poor compared with euchromatin, it still contains transcriptionally active genes. In plants, in addition to heterochromatin located in the centromeric and pericentromeric regions, heterochromatin is located at the nucleolar organizer, at the knobs, and along the maize (Zea mays) B chromosomes. Within plant genomes, the location of potentially active genes has been identified in heterochromatin for example the knob structures and in the pericentromeric region. Yet, while heterochromatin may be gene poor compared with euchromatin, it still contains transcriptionally active genes. The surprising results presented below in Example 5, demonstrate that the methods disclosed herein for site specific targeting DNA recombination between homologous chromosomes in a somatic plant cell, work for both euchromatin and unexpectedly, heterochromatin where recombination is generally suppressed.

In another embodiment, a nuclease disclosed herein is guided to a region within a preselected endogenous target site, wherein said targeting region length comprises about 20 bp. In another embodiment, the targeting region length comprises about 30 bp. In another embodiment, the targeting region length comprises less than 20 bp. In another embodiment, the targeting region length for DSB comprises greater than 20 bp. In some embodiments, a nuclease disclosed herein is guided to a target region in order to create a DSB.

In some embodiments, the preselected endogenous target site comprises the polymorphic allele. In some embodiments, the preselected endogenous target site is adjacent to the polymorphic allele. In some embodiments, the preselected endogenous target site is upstream from the polymorphic allele. In some embodiments, the preselected endogenous target site is downstream from the polymorphic allele.

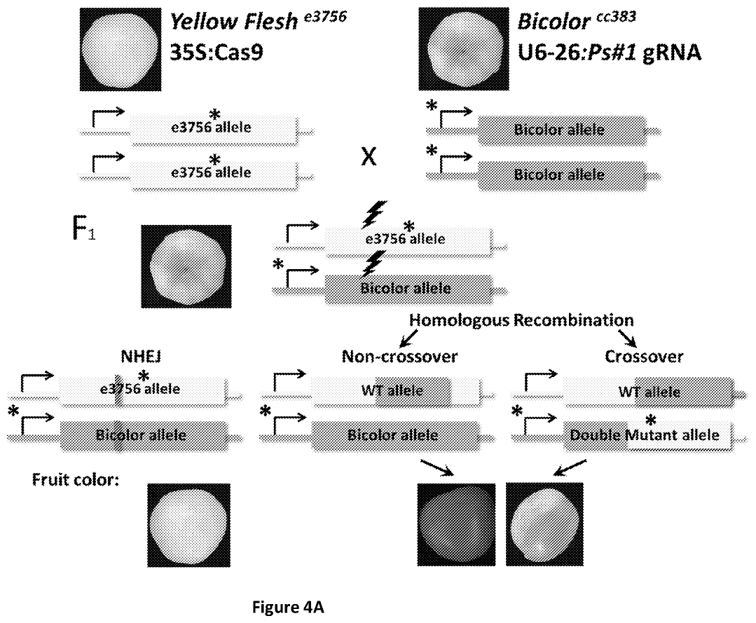

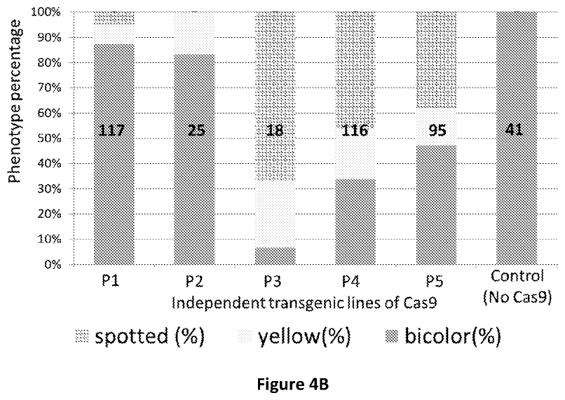

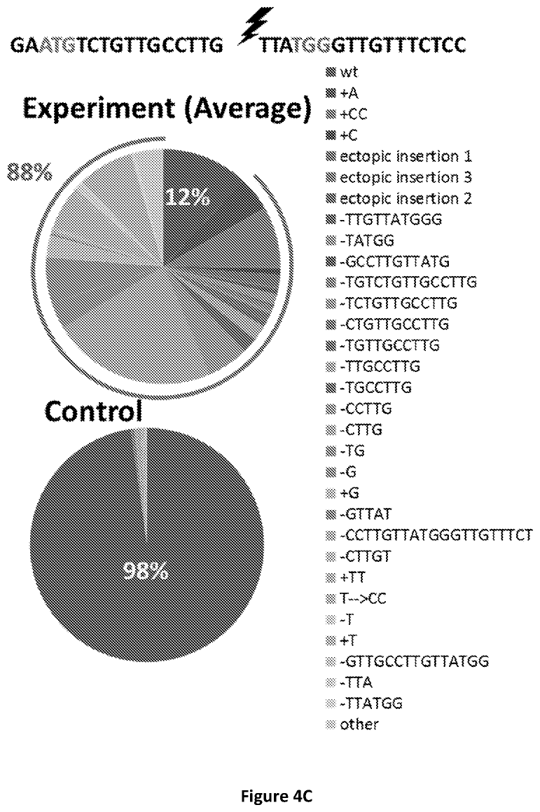

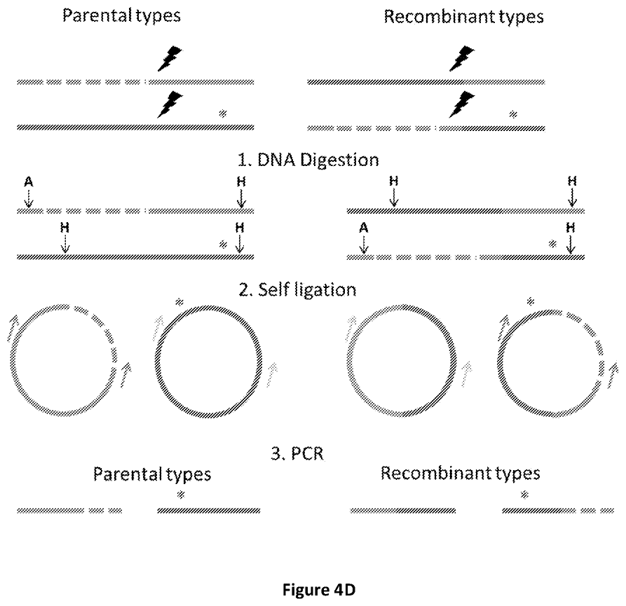

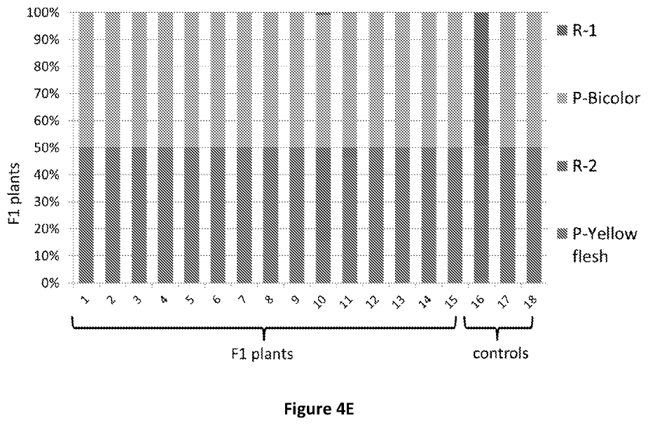

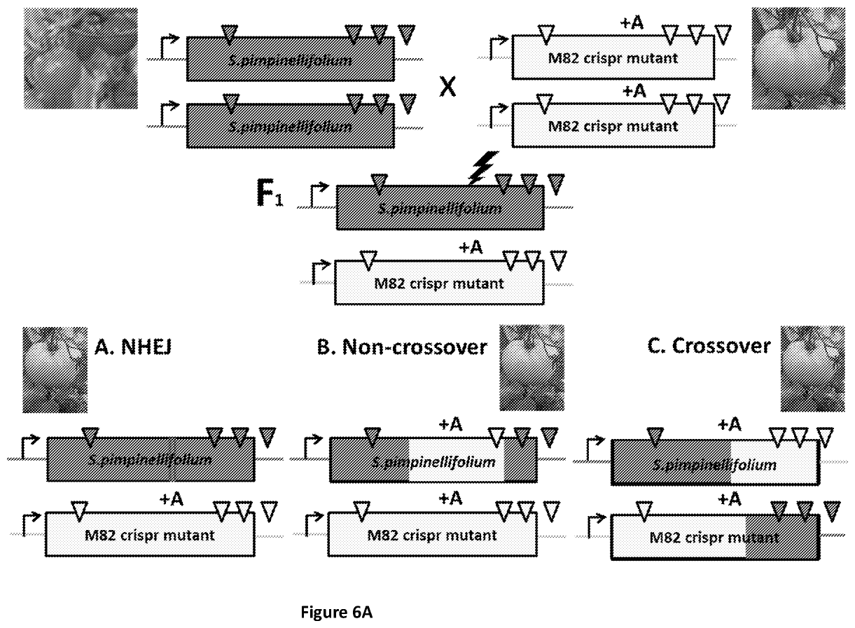

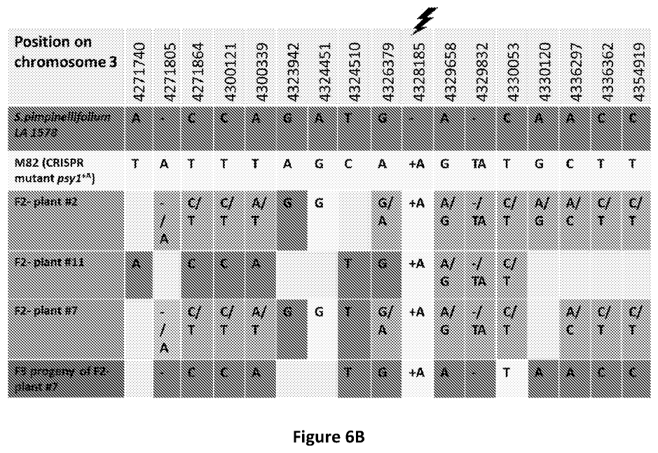

In some embodiments, targeted homologous recombination between homologous chromosomes encompasses exchanges of DNA guided by homologous sequences present on the homologous chromosomes present in the genome of the plant cells and acted on by enzymatic machinery of the cell (FIG. 3; FIG. 4A; FIG. 6A). In one embodiment, the exchange of DNA includes DNA within the preselected endogenous target site. In another embodiment, the exchange of DNA includes but is not limited to DNA within the preselected endogenous target site. In another embodiment, the exchange of DNA includes DNA within the preselected endogenous target site and DNA adjacent to the preselected endogenous target site. In another embodiment, the exchange of DNA includes DNA comprising the entire preselected endogenous target site. In another embodiment, the exchange of DNA includes DNA comprising the entire preselected endogenous target site and DNA adjacent to the preselected endogenous target site. In another embodiment, the exchange of DNA includes DNA comprising only a portion of the preselected endogenous target site. In another embodiment, the exchange of DNA includes DNA comprising only a portion of the preselected endogenous target site and DNA adjacent to the preselected endogenous target site. In another embodiment, the exchange of DNA includes DNA 3′ to the DSB. In another embodiment, the exchange of DNA includes DNA5′ to the DSB.

In some embodiment, a plant cell used in the methods described herein has mutations in genes and or regulatory elements thereof, which are required for the non-homologous end joining (NHEJ) pathway following a DSB. of homologous DNA repair. For example, in some embodiments, a plant cell may have a mutation in a ku gene (e.g., ku70 and or ku80). In some embodiments, a plant cell may have a mutation in lig4. In some embodiments, a plant cell may have a mutation in any gene or regulatory element, wherein the mutation would lead to a decrease NHEJ repair following a DSB.

FIG. 3 schematically presents some embodiments of a method of inducing homologous recombination between homologous chromosomes, for example homologous chromosomes in a plant cell, a plant tissue, or a whole plant. A method of targeted recombination between homologous chromosomes in the genome of a somatic cell, for example a plant cell, comprises three steps: (1) Expression of a nuclease system in the plant cell; (2) Inducing a DNA double-strand break in one or both alleles of a preselected site; and (3) Repairing the DNA via recombination between homologous chromosomes. In one embodiment, disclosed herein is a method of targeting DNA recombination between homologous chromosomes in a somatic plant cell, the method comprising the steps of (a) expressing a nuclease system in the plant cell, wherein said expressed nuclease system is targeted to a preselected endogenous target site comprising polymorphic alleles on the homologous chromosomes, wherein upon expression of the nuclease system the DNA of at least one of said polymorphic alleles is cleaved within said preselected endogenous target site, wherein said nuclease cleaves the DNA creating a double-strand break (DSB) in the DNA of the at least one of the polymorphic alleles; (b) analyzing progeny of said plant cell, or a plant tissue grown from said plant cell, or a plant grown from said cell or a progeny of said plant thereof, for homologous recombination between the homologous chromosomes, wherein the homologous recombination comprises crossover or gene conversion (non-crossover); and (c) selecting a plant cell, plant tissue thereof, plant thereof, or plant progeny thereof comprising DNA comprising said targeted homologous recombination event.

FIG. 3—Step 1: Expression of the nuclease system. The nuclease system to be expressed may comprise any nuclease system capable of targeting a double-stranded cleavage activity to a preselected site in the DNA of at least one allele of the homologous chromosomes.

For example, in some embodiments, a nuclease system used in a method disclosed herein comprises a zinc finger nuclease (ZFN) system, a transcription activator-like effector nuclease (TALEN) system, or a clustered regularly interspaced short palindromic repeats (CRISPR)/CRISPR associated proteins (Cas) system. In other embodiments, a nuclease system used in a method disclosed herein comprises any nuclease system capable of targeting a nuclease capable of double-strand cleavage of DNA to a preselected site on the DNA. In another embodiment, a nuclease system comprises a bacterial Argonaut and a DNA guide. In another embodiment, the double-strand nuclease cleaves the DNA to produce blunt ends. In another embodiment, the double-strand nuclease cleaves the DNA to produce jagged cut ends.

In another embodiment, the double-strand nuclease cleaves the DNA within the polymorphic allele. In another embodiment, the double-strand nuclease cleaves the DNA upstream of the polymorphic allele. In another embodiment, the double-strand nuclease cleaves the DNA downstream of the polymorphic allele. In another embodiment, a nuclease system comprises a zinc finger nuclease (ZFN), wherein the ZFN may be known in the art or newly created to cleave a preselected site. In another embodiment, a nuclease system comprises a transcription activator-like effector nuclease (TALEN), wherein the TALEN may be known in the art or newly created to cleave a preselected site. In another embodiment, a nuclease system comprises a clustered regularly interspaced short palindromic repeats (CRISPR)/CRISPR associated proteins (Cas) system (CRISPR/Cas), wherein the sgRNA and or the Cas may be known in the art or newly created to cleave at a preselected site.

The skilled artisan would appreciate that the terms “single-guide RNA”, “sgRNA”, and “gRNA” are interchangeable having all the same qualities and meanings, wherein an sgRNA may encompass a chimeric RNA molecule which is composed of a CRISPR RNA (crRNA) and trans-encoded CRISPR RNA (tracrRNA). In some embodiments, a crRNA is complementary to a region of DNA within a preselected endogenous target site on at least one of the homologous chromosomes, wherein the crRNA “targets” the CRISPR associated polypeptide (Cas) nuclease protein to the preselected endogenous target site.

In one embodiment, the length of crRNA sequence complementary is 19-22 nucleotides long e.g., 19-22 consecutive nucleotides complementary to the target. In another embodiment, the length of crRNA sequence complementary to the region of DNA is about 15-30 nucleotides long. In another embodiment, the length of crRNA sequence complementary to the region of DNA is about 15, 16, 17, 18, 19, 20, 21, 22, 23, 24, 25, 26, 27, 28, 29, or 30 nucleotides long. In another embodiment, the length of crRNA sequence complementary to the region of DNA is 20 nucleotides long. In one embodiment, the crRNA is located at the 5′ end of the sgRNA molecule. In another embodiment, the crRNA comprises 100% complementation within the preselected target sequence. In another embodiment, the crRNA comprises at least 80% complementation within the preselected target sequence. In another embodiment, the crRNA comprises at least 85% complementation within the preselected target sequence. In another embodiment, the crRNA comprises at least 90% complementation within the preselected target sequence. In another embodiment, the crRNA comprises at least 95% complementation within the preselected target sequence. In another embodiment, the crRNA comprises at least 97% complementation within the preselected target sequence. In another embodiment, the crRNA comprises at least 99% complementation within the preselected target sequence.

In another embodiment, a tracrRNA is 100-300 ribonucleotides long and provides a binding site for the Cas nuclease e.g., a Cas9 protein forming the CRISPR/Cas9 complex.

In one embodiment, the nuclease system comprises a zinc finger nuclease (ZFN) comprising a zinc-finger DNA binding domain and a DNA nuclease cleavage domain, wherein said zinc-finger DNA binding domain binds within said preselected endogenous target site, thereby targeting the DNA nuclease cleavage domain to cleave the DNA within said preselected endogenous target site.

A skilled artisan would appreciate that the terms “zinc finger nuclease” or “ZFN” are interchangeable having all the same meanings and qualities, wherein a ZFN encompasses a chimeric protein molecule comprising at least one zinc finger DNA binding domain operatively linked to at least one nuclease capable of double-strand cleaving of DNA. In one embodiment, a zinc finger nuclease creates a double-stranded break at a preselected endogenous target site. In another embodiment, a zinc finger nuclease comprises a DNA-binding domain and a DNA-cleavage domain, wherein the DNA binding domain is comprised of at least one zinc finger and is operatively linked to a DNA-cleavage domain. In another embodiment, a zinc finger DNA-binding domain is at the N-terminus of the chimeric protein molecule and the DNA-cleavage domain is located at the C-terminus of the molecule. In another embodiment, a zinc finger DNA-binding domain is at the C-terminus of the chimeric protein molecule and the DNA-cleavage domain is located at the N-terminus of the molecule. In another embodiment, a zinc finger binding domain encompasses the region in a zinc finger nuclease that is capable of binding to a target locus, for example a preselected endogenous target site as disclosed herein. In another embodiment, a zinc finger DNA-binding domain comprises a protein domain that binds to a preselected endogenous target site on at least one homologous chromosome. In another embodiment, a zinc finger DNA-binding domain comprises a protein domain that binds to a polymorphic allele on at least one homologous chromosome. In another embodiment, a zinc finger DNA-binding domain comprises a protein domain that binds to a preselected endogenous target site on both homologous chromosomes. In another embodiment, a zinc finger DNA-binding domain comprises a protein domain that binds to polymorphic alleles on both homologous chromosomes.

The skilled artisan would appreciate that the term “chimeric protein” is used to describe a protein that has been expressed from a DNA molecule that has been created by operatively joining two or more DNA fragments. The DNA fragments may be from the same species, or they may be from a different species. The DNA fragments may be from the same or a different gene.

The skilled artisan would appreciate that the term “DNA cleavage domain” of a ZFN encompasses the region in the zinc finger nuclease that is capable of breaking down the chemical bonds between nucleic acids in a nucleotide chain. Examples of proteins containing cleavage domains include restriction enzymes, topoisomerases, recombinases, integrases and DNAses

In one embodiment, a nuclease system comprises a transcription activator-like effector nuclease (TALEN) system comprising a TAL effector DNA binding domain and a DNA cleavage domain, wherein said TAL effector DNA binding domain binds within said preselected endogenous target site, thereby targeting the DNA cleavage domain to cleave the DNA within said preselected endogenous target site.

A skilled artisan would appreciate that the terms “transcription activator-like effector nuclease”, “TALEN”, and “TAL effector nuclease” may be used interchangeably having all the same meanings and qualities, wherein a TALEN encompasses a nuclease capable of recognizing and cleaving its target site, for example a preselected endogenous target site as disclosed herein. In another embodiment, a TALEN comprises a fusion protein comprising a TALE domain and a nucleotide cleavage domain. In another embodiment, a TALE domain comprises a protein domain that binds to a nucleotide in a sequence-specific manner through one or more TALE-repeat modules. In another embodiment, a TALE domain comprises a protein domain that binds to a preselected endogenous target site on at least one homologous chromosome. In another embodiment, a TALE domain comprises a protein domain that binds to a polymorphic allele on at least one homologous chromosome. In another embodiment, a TALE domain comprises a protein domain that binds to a preselected endogenous target site on both homologous chromosomes. In another embodiment, a TALE domain comprises a protein domain that binds to polymorphic alleles on both homologous chromosomes.

In one embodiment, a TALE domain comprises at least one of the TALE-repeat modules. In another embodiment, a TALE domain comprises from one to thirty TALE-repeat modules. In another embodiment, a TALE domain comprises more than thirty repeat modules.

In another embodiment, a TALEN fusion protein comprises an N-terminal domain, one or more of TALE-repeat modules followed by a half-repeat module, a linker, and a nucleotide cleavage domain.

In one embodiment, a nuclease system comprises a CRISPR/Cas system. In another embodiment, a CRISPR/Cas system comprises a Cas nuclease and a gRNA molecule, wherein said gRNA molecule binds within said preselected endogenous target site thereby guiding said Cas nuclease to cleave the DNA within said preselected endogenous target site.

In some embodiments, a CRISPR/Cas system comprise an enzyme system including a guide RNA sequence (“gRNA” or “sgRNA”) that contains a nucleotide sequence complementary or substantially complementary to a region of a target polynucleotide, for example a preselected endogenous target site, and a protein with nuclease activity.

In another embodiment, a CRISPR/Cas system comprises a Type I CRISPR-Cas system, or a Type II CRISPR-Cas system, or a Type III CRISPR-Cas system, or derivatives thereof. In another embodiment, a CRISPR-Cas system comprises an engineered and/or programmed nuclease systems derived from naturally accruing CRISPR-Cas systems. In another embodiment, a CRISPR-Cas system comprises engineered and/or mutated Cas proteins. In another embodiment, a CRISPR-Cas system comprises engineered and/or programmed guide RNA.

A skilled artisan would appreciate that the term “guide RNA” encompasses a RNA containing a sequence that is complementary or substantially complementary to a region of a target DNA sequence. A guide RNA may contain nucleotide sequences other than the region complementary or substantially complementary to a region of a target DNA sequence, for example a preselected endogenous target site. In another embodiment, a guide RNA comprises a crRNA or a derivative thereof. In another embodiment, a guide RNA comprises a crRNA: tracrRNA chimera.

In another embodiment, a gRNA molecule comprises a domain that is complementary to and binds to a preselected endogenous target site on at least one homologous chromosome. In another embodiment, a gRNA molecule comprises a domain that is complementary to and binds to a polymorphic allele on at least one homologous chromosome. In another embodiment, a gRNA molecule comprises a domain that is complementary to and binds to a preselected endogenous target site on both homologous chromosomes. In another embodiment, a gRNA molecule comprises a domain that is complementary to and binds to polymorphic alleles on both homologous chromosomes.

Cas enzymes comprise RNA-guided DNA endonuclease able to make double-stranded breaks (DSB) in DNA. The term “Cas enzyme” may be used interchangeably with the terms “CRISPR-associated endonucleases” or “CRISPR-associated polypeptides” having all the same qualities and meanings. In one embodiment, a Cas enzyme is selected from the group comprising Cas1, Cas1B, Cas2, Cas3, Cas4, Cas5, Cas6, Cas7, Cas8, Cas9, Cas10, C2c1, CasX, NgAgo, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, or homologs thereof, or modified versions thereof. In another embodiment, a Cas enzyme comprises Cas9. In another embodiment, a Cas enzyme comprises Cas1. In another embodiment, a Cas enzyme comprises Cas1B. In another embodiment, a Cas enzyme comprises Cas2. In another embodiment, a Cas enzyme comprises Cas3. In another embodiment, a Cas enzyme comprises Cas4. In another embodiment, a Cas enzyme comprises Cas5. In another embodiment, a Cas enzyme comprises Cas6. In another embodiment, a Cas enzyme comprises Cas7. In another embodiment, a Cas enzyme comprises Cas8. In another embodiment, a Cas enzyme comprises Cas10. In another embodiment, a Cas enzyme comprises Cpf1. In another embodiment, a Cas enzyme comprises Csy1. In another embodiment, a Cas enzyme comprises Csy2. In another embodiment, a Cas enzyme comprises Csy3. In another embodiment, a Cas enzyme comprises Cse1. In another embodiment, a Cas enzyme comprises Cse2. In another embodiment, a Cas enzyme comprises Csc1. In another embodiment, a Cas enzyme comprises Csc2. In another embodiment, a Cas enzyme comprises Csa5. In another embodiment, a Cas enzyme comprises Csn2. In another embodiment, a Cas enzyme comprises Csm2. In another embodiment, a Cas enzyme comprises Csm3. In another embodiment, a Cas enzyme comprises Csm4. In another embodiment, a Cas enzyme comprises Csm5. In another embodiment, a Cas enzyme comprises Csm6. In another embodiment, a Cas enzyme comprises Cmr1. In another embodiment, a Cas enzyme comprises Cmr3. In another embodiment, a Cas enzyme comprises Cmr4. In another embodiment, a Cas enzyme comprises Cmr5. In another embodiment, a Cas enzyme comprises Cmr6. In another embodiment, a Cas enzyme comprises Csb1. In another embodiment, a Cas enzyme comprises Csb2. In another embodiment, a Cas enzyme comprises Csb3. In another embodiment, a Cas enzyme comprises Csx17. In another embodiment, a Cas enzyme comprises Csx14. In another embodiment, a Cas enzyme comprises Csx10. In another embodiment, a Cas enzyme comprises Csx16, CsaX. In another embodiment, a Cas enzyme comprises Csx3. In another embodiment, a Cas enzyme comprises Csx1, Csx15, Csf1. In another embodiment, a Cas enzyme comprises Csf2. In another embodiment, a Cas enzyme comprises Csf3. In another embodiment, a Cas enzyme comprises Csf4. In another embodiment, a Cas enzyme comprises Cpf1. In another embodiment, a Cas enzyme comprises C2c1. In another embodiment, a Cas enzyme comprises CasX. In another embodiment, a Cas enzyme comprises NgAgo. In another embodiment, a Cas enzyme is Cas homologue. In another embodiment, a Cas enzyme is a Cas orthologue. In another embodiment, a Cas enzyme is a modified Cas enzyme. In another embodiment, a Cas enzyme is any CRISPR-associated endonucleases known in the art.

In one embodiment, a somatic plant cell is transformed in order to expresses a nuclease system or a component thereof. In another embodiment, at least one parent cell of a somatic plant cell is transformed in order to express a nuclease system or a component thereof. In another embodiment, one parent of the somatic plant cell is transformed to express a nuclease system or a component thereof. In another embodiment, each parent of the somatic plant cell is transformed to express a component of a nuclease system. In another embodiment, one parent is transformed to express both components of a nuclease system.

In some embodiments, following a homologous recombination event the progeny cells, tissue and or plants no longer contains a target for the nuclease system. (For example see FIG. 6A, wherein additional DSB do not occur as the preselected endogenous target site no long exists. Sequencing of the DNA has shown that there are no additional DSB events.) In some embodiments, following homologous recombination induced by a DSB at the endogenous target site, the sequence of the endogenous target site has been altered by the HR. In some embodiments, following homologous recombination induced by the DSB, the nuclease system lacks functionality. In some embodiments, following homologous recombination induced by the DSB, the nuclease system lacks the ability to target a nuclease activity to the endogenous target site.

In another embodiment, a somatic plant cell is comprised within a hybrid plant or within a heterozygous plant, wherein said cell comprises polymorphic alleles. In another embodiment, a somatic plant cell comprises an existing hybrid or heterozygous plant cell having polymorphic alleles at the preselected endogenous target site. In another embodiment, a somatic plant cell comprising polymorphic alleles is transformed to express a nuclease system.

In one embodiment, a somatic cell is transformed with a DNA encoding a nuclease system or a component thereof. In another embodiment, an isolated tissue of a plant is transformed with a DNA encoding a nuclease system or a component thereof. In another embodiment, a parent cell is transformed with a DNA encoding a nuclease system or a component thereof. In another embodiment, a both parent cells are transformed with a DNA encoding a nuclease system or a component thereof.

In one embodiment, a somatic cell is transformed with a RNA encoding a nuclease system or a component thereof. In another embodiment, an isolated tissue of a plant is transformed with a RNA encoding a nuclease system or a component thereof. In another embodiment, a parent cell is transformed with a RNA encoding a nuclease system or a component thereof. In another embodiment, a both parent cells are transformed with a RNA encoding a nuclease system or a component thereof.

In one embodiment, a somatic cell is transformed with a polypeptide comprising a nuclease system or a component thereof. In another embodiment, an isolated tissue of a plant is transformed with a polypeptide comprising a nuclease system or a component thereof. In another embodiment, a parent cell is transformed with a polypeptide comprising a nuclease system or a component thereof. In another embodiment, a both parent cells are transformed with a polypeptide comprising a nuclease system or a component thereof.

In some embodiments, transformation of a plant cell or of an isolated plant tissue is by any method known in the art. In another embodiment, transformation result in transient expression. In another embodiment, transformation results in stable expression. In another embodiment, stable transformation is by the method of Agrobacterium. In another embodiment, transformation comprises direct transformation. In another embodiment, direct transformation comprises the use of polyethylene glycol (PEG). In another embodiment, direct transformation comprises the use of electroporation via bombardment.

In some embodiments, DNA introduced into a plant cell, for example DNA encoding a nuclease system may be eliminated from the plant genome by genetic segregation. Alternatively, in some embodiments, the DNA is expressed transiently and thus does not remain in the plant cell.

FIG. 3 box a illustrates that transformation may be for both parents, wherein for example, each of them being transformed with one component of the nuclease, for example a CRISPR/Cas nuclease, that becomes active upon hybridization. Both nuclease components can be introduced in one of the parent (FIG. 3 box b). In the embodiment illustrated in FIG. 3 box b, the nuclease should be “silent” and become activated in the hybrid (using an inducible system). In another embodiment illustrated in FIG. 3 box b, the nuclease system may be targeted at the allele of the second parent while it does not cleave the allele of the transformed parental plant cell, thus it becomes active in an allele-specific manner in the hybrid. In yet another embodiment, transformation can be carried on an existing hybrid or an heterozygote plant (FIG. 3 box c) with all nuclease components.

In some embodiment, the activity or activation of a nuclease system is inducible. In another embodiment, an inducible nuclease system may utilize inducible promotors. In another embodiment, an inducible promoter may be tissue specific. In another embodiment, an inducible promoter may be induced (turned on) under conditions stressful to a plant cell or tissue. In some embodiment, the activity or activation of a nuclease system is constitutive. In another embodiment, the activity or activation may be tissue specific. In another embodiment, expression of the nuclease system or a portion thereof is regulated. In another embodiment, a constitutive promoter is used to express all components of a nuclease system disclosed herein. In another embodiment, any regulatatable plant promoter known in the art is used to express all components of a nuclease system disclosed herein. In another embodiment, any regulatable plant promoter known in the art is use to express at least one component of a nuclease system disclosed herein. In another embodiment, any regulatatable promoter known in the art and functional in the plant cell is used to express all components of a nuclease system disclosed herein. In another embodiment, any regulatable promoter known in the art and functional in the plant cell is use to express at least one component of a nuclease system disclosed herein.

In some embodiments, a somatic plant cell comprises a cell from the progeny of crossing two cultivar plant cells or plants, wherein said parental plant cells each comprise a polymorphic allele compared with said mate at said preselected endogenous target site. In some embodiments, a somatic plant cell comprises a cell from a plant progeny of a cross between two polymorphic parental lines, which creates a hybrid plant, wherein said parental plant lines each comprise a polymorphic allele at said preselected endogenous target site, and wherein only one of the parental lines comprises said nuclease system. In some embodiments, a somatic plant cell comprises a cell from a plant progeny of a cross between two polymorphic parental lines, which creates a hybrid plant, wherein said parental plant lines each comprise a polymorphic allele at said preselected endogenous target site, and wherein each of the parental lines comprises a component of the nuclease system.

A skilled artisan would appreciate that the term “progeny”, as used herein, encompasses the offspring of selfing or a cross and includes direct first generation progeny (e.g., F1), as well as later generations (e.g., F2, F3, etc), as well as backcross generations, for example for 1-3 generations. In one embodiment, progeny comprise any generation of plant or plant cell derived from the plant, where induced targeted homologous recombination, as disclosed herein, has occurred.

In another embodiment, progeny comprise an F1 generation. In another embodiment, progeny comprise an F2 generation. In another embodiment, progeny comprise an F3 generation. In another embodiment, progeny comprise an F4 generation. In another embodiment, progeny comprise multiple generations selected from F1 generation-F4 generations. In another embodiment, progeny comprise a 1^{st} backcross generation. In another embodiment, progeny comprise a 2^{nd} backcross generation. In another embodiment, progeny comprise a 3^{rd} backcross generation. In another embodiment, progeny comprise a 4^{th} backcross generation. In another embodiment, progeny comprise multiple backcross generations selected from 1^{st}-4^{th} backcross generations.

In another embodiment, one of said parent somatic plant cells comprises said nuclease system, and wherein the DNA cleaving activity of said nuclease system is targeted to the polymorphic allele present in the other parent plant cell that does not comprise said nuclease system. In another embodiment, one of said parent somatic plant cells comprises a Cas nuclease and the other of said parent somatic plant cells comprises a gRNA molecule, wherein said gRNA molecule binds within said preselected endogenous target site thereby guiding said Cas nuclease to cleave the DNA within said preselected endogenous target site. In another embodiment, one of said parent somatic plant cells comprises a Cas9 nuclease and the other of said parent somatic plant cells comprises a gRNA molecule, wherein said gRNA molecule binds within said preselected endogenous target site thereby guiding said Cas9 nuclease to cleave the DNA within said preselected endogenous target site.

In another embodiment, a somatic plant cell comprises a cell from the progeny of crossing a plant cell from a cultivar with a wild-type plant cell, wherein said parental plant cells each comprise a polymorphic allele compared with said mate at said preselected endogenous target site, and wherein said plant cell from the cultivar comprises said nuclease system. In another embodiment, wherein the somatic plant cell comprises a cell from the progeny of crossing a plant cell from a cultivar with a wild-type plant cell, the DNA cleaving activity of said nuclease system occurs solely on the polymorphic allele present in wild-type parent plant cell. In another embodiment, the nuclease system comprises a ZFN, and wherein the DNA cleaving activity of said nuclease system occurs solely on the polymorphic allele present in wild-type parent plant cell. In another embodiment, the nuclease system comprises TALEN wherein the DNA cleaving activity of said nuclease system occurs solely on the polymorphic allele present in wild-type parent plant cell.

In another embodiment, a somatic plant cell having polymorphic alleles is created by any means known in the art. In another embodiment, a somatic plant cell having polymorphic alleles is a plant cell obtained from a cultivar.

FIG. 3—Step 2: Induction of DNA double-strand break (DSB), which is represented as a yellow lightning bolt. In some embodiments, a DSB occurs in one allele of said polymorphic alleles. In some embodiments, a DSB occurs in both alleles of said polymorphic alleles in the case of a diploid. In some embodiments, a DSB occurs in only one allele of said polymorphic alleles in the case of a diploid or cell with higher ploidy, e.g., a triploid. In some embodiments, a DSB occurs in two alleles of said polymorphic alleles in the case of a diploid or cell with higher ploidy, e.g., a triploid. In some embodiments, a DSB occurs in each allele of said polymorphic alleles, for example two DSBs in a diploid, three DSB is a triploid etc.

While the Examples provided below and FIG. 1, illustrated DSB cleavage using the CRISPR/Cas system, one skilled in the art would appreciate that these examples are not limiting and that any other site-specific nuclease can be used, for example a ZFN or a TALEN. In another embedment, induction of a DSB is with a ZFN system. In another embodiment, induction of a DSB is with a TALEN system. In another embodiment, induction of a DSB is with a CRISPR/Cas system. In another embodiment, induction of a DSB is with any nuclease system that can be targeted to a preselected endogenous target site and that can make a DSB in the DNA.

In some embodiments, a DSB can be induced in any plant tissues or cells and any stages of cell cycle. For example, in one embodiment, a constitutive promoter can be used to activate the nuclease so that DSB-induction can occur as early as in the zygote of the hybrid. In another embodiment, a DSB occurs at an early part of the plant development of a somatic tissue. In another embodiment, a DSB occurs at a late part of the development of a somatic tissue. In another embodiment, a DSB occurs between an early and late part of the development of a somatic tissue.

In another embodiment, a protoplast is transformed by a DNA or RNA vector or by a complex of purified protein and gRNA or by a purified protein in the case of a single component nuclease system, for example ZFN or TALEN.

In one embodiment, inducing comprises constitutive induction. In another embodiment, inducing comprises non-constitutive induction. In another embodiment, inducing comprises tissue-specific induction. In another embodiment, inducing comprises condition-specific induction. In another embodiment, inducing comprises cell-cycle dependent induction. In another embodiment, inducing comprises constitutive induction, non-constitutive induction, tissue specific induction, or cell-cycle specific induction, or any combination thereof.

FIG. 3—Step 3 Within the cell, an induced DSB may be repaired via non-homologous end joining (FIG. 1) or via homologous recombination (HR) between homologous chromosomes (endogenous repair template FIG. 1, FIG. 2, and FIG. 3). In some embodiments, a DSB is repaired via non-homologous end joining (NHEJ). In other embodiments, a DSB is repaired via homologous recombination (HR). Unexpectedly, as shown in the Examples below, use of a method of targeted recombination between homologous chromosomes as disclosed herein, results in a significantly higher frequency of HR repair than would be expected to occur naturally, in the absence of DSB induction.

In one embodiment, the outcome of DSB repair comprises Gene Conversion (also known as non-crossover). In another embodiment, the outcome of DSB repair comprises Crossover. The DSB repair HR products can be identified by different analysis, for example by genetic markers, by the change in SNPs pattern, by DNA sequencing methods, by loss of heterozygosity (LOH) phenotypes, or by phenotypic, or by any other marker, or by a combination thereof.

In order to determine and select plant cells wherein HR has occurred, progeny of said cells may be analyzed. Analysis, may in one embodiment, comprise analysis of progeny cells. In another embodiment, analysis comprises analyzing plant cells generated from said somatic cell or progeny plant or plant tissue thereof. In another embodiment, analysis comprises analyzing a plant tissue generated from said somatic cell or progeny plant or plant tissue thereof. In another embodiment, analysis comprises analyzing a plant tissue. In another embodiment, analysis comprises analyzing a plant progeny of said somatic cell, or a tissue or cell thereof. Any type of cells may be screened, depending on the plant system and the desired application. For example in sexually reproducing plants, seeds, grains, fruits or even pollen grains can be screened for the identification of HR repaired alleles that have been inherited to next generation. In trees or vegetative reproducing plants, protoplasts, calli, leaves, stems etc. can be screened and then regenerated. In some embodiments, analyzing said plant comprises analyzing a portion of said plant or a progeny thereof comprising a leaf, a stem, a bud, a fruit, a seed, or pollen, or any combination thereof. As a result the method is applicable to any plant species.

In some embodiments, a somatic plant cell is comprised within a plant tissue or a plant. A skilled artisan would appreciate that the term “plant” encompasses any species of woody, herbaceous, perennial or annual plant. In one embodiment, a somatic plant cell disclosed herein comes from a plant comprising any species of woody, herbaceous, perennial or annual plant. The term “plant” may also encompass a plurality of plant cells that are largely differentiated into a structure that is present at a stage of the plant development capable of producing crop.

In one embodiment, a somatic cell disclosed herein comes from a crop plant. In some embodiment, a somatic plant cell comprises a crop plant cell. A skilled artisan would appreciate that the term “crop plant” encompasses a plant with at least one part having commercial value. In one embodiment, a crop plant comprise plants producing edible fruit (including vegetables), plants producing grains (as a food, feed and for oil production), plants producing flowers and ornamental plants, legumes, root crops, tuber crops, or leafy crops and the like.

In one embodiment, a plant comprises an alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, Clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugar cane, sunflower, sweet potato, sweetgum, switchgrass, tangerine, tea, tobacco, tomato, triticale, turf, turnip, a vine, watermelon, wheat, yams, and zucchini.

In some embodiment, methods of targeted recombination between homologous chromosomes of a somatic plant cell comprise methods for precise breeding of crops. In some embodiments, methods of making a somatic plant cell comprising DNA comprising a targeted HR event (e.g., a gene conversion or crossover event) using targeted recombination between homologous chromosomes comprises methods for precise breeding of crops. Methods disclosed herein may precisely introduce qualities and or traits not previously present the somatic plant cell, tissues thereof, plants thereof, or progeny thereof. Such qualities being present, for example in one of the parent cells of said somatic cell. For example, using methods disclosed herein a farmer or plant breeder could create a hardier plant or a plant resistant to naturally hazards such as pests, pathogens, drought, or poor soil conditions, or any combination thereof. In other embodiment, methods disclosed herein could produce a crop, for example a fruit or vegetable having increased nutritional properties, or increased resistance to pests or pathogens, or more stable over time in order to improve the quality of produce transported from one place to another. In some embodiments, a desired quality or trait is present in a wild-type population of the plant. In some embodiments, a desired quality or trait is present is a cultivar population of the plant. In some embodiments, a desired quality or trait is present in a wild-type species of the plant but not a corresponding cultivar. In some embodiments, a desired quality or trait is present in one cultivar of a species of the plant but not a corresponding cultivar.

In some embodiments, a somatic plant cell comprising DNA comprising said HR event, or a plant tissue comprising said cell comprising DNA comprising said HR event, or a plant comprising said cell or a progeny plant thereof comprising DNA comprising said HR event, or fruit derived from a plant comprising said cell or progeny plant thereof comprising DNA comprising said HR event, or seeds derived from a plant comprising said cell or progeny plant thereof comprising DNA comprising said HR event, or any combination thereof has increased drought resistance, increased resistance to pests, increased resistance to pathogens, improved nutrient content, improved growth parameters, or any combination thereof as compared to a control plant cell, plant or progeny thereof. In one embodiment, a control plant cell, plant or progeny thereof is a parent cell, plant or progeny thereof. In another embodiment, a control plant cell, plant or progeny thereof is a somatic cell, plant or progeny thereof wherein said DSB does not or did not occur.

In some embodiments, a preselected endogenous target site targeted in methods disclosed herein comprises DNA comprising a locus, a part of a locus, a gene, a part of a gene, a regulatory upstream sequence of a gene, a regulatory downstream sequences of a gene, an upstream sequence of a gene, a downstream sequence of a gene, or any combination thereof, and wherein expression or lack thereof of said gene affects growth, drought resistance, resistance to pests, resistance to pathogens, or nutrient content, or any combination thereof of said plant cell comprising DNA comprising the targeted HR event, or a progeny thereof compared with a control plant cell or progeny thereof, plant tissue, plant or progeny thereof.

In some embodiment, selected progeny of step (e) are selected from the group comprising F₁, F₂, F₃, F₄, backcross 1^{st} generation, backcross 2^{nd} generation, backcross 3^{rd} generation, and backcross 4^{th} generation.

In some embodiments, a method disclosed herein produces a somatic plant cell comprising DNA comprising the targeted HR event, or a plant tissue comprising said cell comprising DNA comprising the targeted HR event, or a plant comprising said cell comprising DNA comprising the targeted HR event or a progeny plant thereof comprising DNA comprising the targeted HR event, or fruit derived from a plant comprising said cell comprising DNA comprising the targeted HR event or progeny plant thereof comprising DNA comprising the targeted HR event, or seeds derived from a plant comprising said cell comprising DNA comprising the targeted HR event or progeny plant thereof comprising DNA comprising the targeted HR event, or any combination thereof, wherein the cell, tissue, plant, or progeny thereof has increased drought resistance, increased resistance to pests, increased resistance to pathogens, improved nutrient content, improved growth parameters, or any combination thereof as compared to a control plant cell, plant tissue, plant or progeny thereof, fruit, or seed.

### EXAMPLES

### Materials and Methods—For Examples 1-4

Plant Material

All tomato plants were grown in greenhouse conditions with temperature ranging between 18 to 25° C. The tomato (S. lycopersicum) mutant line of yellow flesh e³⁷⁵⁶, Bicolor^{cc383} M82 and Solanum pimpinellifolium^{LA1578} were kindly provided by the labs of Prof. Joseph Hirschberg and Prof. Daniel Zamir at the Hebrew university of Jerusalem (Kachanovsky, D. E., Filler, S., Isaacson, T. & Hirschberg, J. Epistasis in tomato color mutations involves regulation of phytoene synthase 1 expression by cis-carotenoids. Proc. Natl. Acad. Sci. U.S.A 109, 19021-6 (2012)).

Plasmids and Plant Transformation

The 35s:Cas9 and u6-26:sgRNA constructs were previously cloned by Ross A. Johnson (Johnson, R. A., Gurevich, V., Filler, S., Samach, A. & Levy, A. A. Comparative assessments of CRISPR-Cas nucleases' cleavage efficiency in planta. Plant Mol. Biol. 87, 143-56 (2015)). The primers used for construction of ps #1 sgRNA ps #2f targets are specified in the primers list in Johnson et al., (2015) (ibid) and are presented here in Table 1.

TABLE 1
Primers for sgRNA Targets
Primers for sgRNA targets:

| | | |
|---|---|---|
| Ps#1 sgRNA F | attgGAATGTCT | SEQ ID NO. 1 |
| | GTTGCCTTGTTA | |
| Ps#1 sgRNA R | aaacTAACAAGG | SEQ ID NO. 2 |
| | CAACAGACATT | |
| Ps#2 sgRNA F | attgGAGCGTAT | SEQ ID NO. 3 |
| | ATAATGCTGCTT | |
| Ps#2 sgRNA R | aaa cAAG CAG | SEQ ID NO. 4 |
| | CATTATATAC GCT | |

The DNA sequence encoding the sgRNA used in the Examples are presented in Table 2.

TABLE 2
sgRNA DNA sequence encoding the sgRNA sequence

| | | |
|---|---|---|
| gRNA molecule | ggagcgtatat aatgctgctt | SEQ ID |
| for Allele- | gttttagagc tagaaatagc | NO: 61 |
| specific DSB | aagttaaaat aaggctagtc | |
| induction and | cgttatcaac ttgaaaaagt | |
| allele dependent | ggcaccgagt cggtgctttt | |
| repair | ttt | |
| Ps#1 gRNA | ggaatgtctgt tgccttgtta | SEQ ID |
| | gttttagagc tagaaatagc | NO. 68 |
| | aagttaaaat aaggctagtc | |
| | cgttatcaac ttgaaaaagt | |
| | ggcaccgagt cggtgctttt | |
| | ttt | |

All tomato lines were transformed by Agrobacterium tumefaciens GV3101 with cotyledon transformation according to McCormick (McCormick, S. in Plant Tissue Culture Manual 311-319 (Springer Netherlands, 1991). doi:10.1007/978-94-009-0103-2_17).

Inverse PCR for Homologous Recombination (HR) detection

DNA samples for the Inverse PCR assay were extracted using a DNA purification kit (MACHEREY-NAGEL®). For each plant 300 ng of DNA from leaves sample or control plasmid were treated separately: first, they were incubated over night with 10×FD buffer, ApaLI (ThermoFisher scientific) and HindIII-HF (New England BioLabs®). After 20 minutes of 80° C. inactivation, 150 ng of the digested fragments were blunted with T4 DNA polymerase (New England)BioLabs® for 2 hours at room temperature. The T4 DNA polymerase was inactivated at 75° C. for 10 minutes and the linear DNA was self ligated with Quick T4 DNA ligase (New England)BioLabs® for 30 minutes at room temperature. Control plasmids were diluted 1:10,000 with DDW and mixed together for mimicking “heterozygosity”. Then all samples were amplified by 18 cycles of PCR with Phusion® High-Fidelity DNA polymerase (New England)BioLabs® (for primers see Table 3).

TABLE 3
Primers for inverse PCR Homologous
Recombination Detection
Primers for inverse PCR HR detection
(allele specific primers):

| | | |
|---|---|---|
| a 3756 bic hr f | tcagC TATG C | SEQ ID NO. 5 |
| | TAATGACTCC CGAG | |
| a bic hr r | agtcCATTCTCTA | SEQ ID NO. 6 |
| | TTCCGCATAGTGA | |
| a 3756 r | tga cAAC CGACC | SEQ ID NO. 7 |
| | TAAATCGATC C G | |
| b bic hr r | actgCATTCTCTA | SEQ ID NO. 8 |
| | TTCCGCATAGTGA | |
| b3756r | gactactgAAC C | SEQ ID NO. 9 |
| | GACCTAAATCGATCC G | |

The primers for this assay were designed for allele specific amplification. Samples were pooled and sequenced by high-throughput sequencing.

For the cloning of the synthetic crossover—control plasmids, two PCR fragments were amplified from yellow flesh e³⁷⁵⁶ and Bicolor^{cc383} DNA samples using Phusion® High-Fidelity DNA polymerase (New England)BioLabs® (for primers sequence see Table 4) and then cloned using the GoldenBraid cloning system (Sarrion-Perdigones, A. et al. GoldenBraid: An Iterative Cloning System for Standardized Assembly of Reusable Genetic Modules. PLoS One 6, e21622 (2011)).

TABLE 4
Primers for Synthetic Crossover-
control plasmids
Primers for synthetic crossover-
control plasmids:

| | | |
|---|---|---|
| pupd_y1_f | gcgccgtctcgctcgtactCG | SEQ ID NO. 10 |
| | AACGAGGGTCAT | |
| pupd_y1_r | gcgccgtctc gctcgagcgc | SEQ ID NO. 11 |
| | cataattgga acactcatca | |
| | a | |
| pupd_ps_f | gcgccgtctcgctcgggagCA | SEQ ID NO. 12 |
| | ACCTTATTTTGT | |
| pupd_ps_r | gcgccgtctcgctcgagtaCA | SEQ ID NO. 13 |
| | ACATATCAAAAT | |

First, each of the four amplicons was cloned into the pUPD plasmid. Then pUPD2 plasmid with “ps” segment from yellow flesh e³⁷⁵⁶ was cloned with pUPD2 plasmid with “y1” segment from Bicolor^{cc383} into pDGB3_alpha1 plasmid. In parallel, pUPD2 plasmid with “ps” segment from Bicolor^{cc383} was cloned with pUPD2 plasmid with “y1” segment from yellow flesh e³⁷⁵⁶ into pDGB3_alpha1 plasmid. These two “synthetic allele” plasmids were pooled together and were subjected to inverse PCR process and sequencing. The DNA sequence of these “synthetic allele” plasmids is presented in Table 5.

TABLE 5
Plasmids for Synthetic Crossover Control
Plasmids for synthetic crossover control (inverse PCR assay)

| | |
|---|---|
| R1 plasmid | SEQ ID NO. 62 |
| R2 plasmid | SEQ ID NO. 63 |

DNA Amplification and Sequencing

DNA samples for high-throughput sequencing were amplified using Phusion® High-Fidelity DNA polymerase (New England BioLabs®) and 18 PCR cycles (for specific primers of each experiment see Table 6).

TABLE 6
Primers for High Throughput Sequencing
Primers for NHEJ High throughput sequencing

| | | |
|---|---|---|
| psy1 t1 htp f | GGTTTGCCTGTCTGT | SEQ ID NO. 14 |
| | GGTCT | |
| psy1 t1 htp r1 | agtcCCATGAAACTT | SEQ ID NO. 15 |
| | GTC C CATTTG | |
| psy1 t1 htp r2 | tcagC | SEQ ID NO. 16 |
| | CATGAAACTTGTC C | |
| | CATTTG | |
| psy1 t1 htp r3 | actgC | SEQ ID NO. 17 |
| | CATGAAACTTGTC C | |
| | CATTTG | |
| psy1 t1 htp r4 | tgacCCATGAAACTT | SEQ ID NO. 18 |
| | GTCCCATTTG | |
| psy1 t1 htp r5 | gactCCATGAAACTT | SEQ ID NO. 19 |
| | GTCCCATTTG | |
| psy1 t1 htp r6 | ctgaCCATGAAACTT | SEQ ID NO. 20 |
| | GTCCCATTTG | |
| nhej_psy1_t2_r | GCCTAAATACGGCAC | SEQ ID NO. 21 |
| | TTCCA | |
| a_nhej_psy1_t2_f | agtcGTATCGCCCCT | SEQ ID NO. 22 |
| | GAATCAAAG | |
| b_nhej_psy1_t2_f | tcagGTATCGC C | SEQ ID NO. 23 |
| | CCTGAATCAAAG | |
| c_nhej_psy1_t2_f | tgacGTATCGCCCCT | SEQ ID NO. 24 |
| | GAATCAAAG | |
| d_nhej_psy1_t2_f | actgGTATCGCCCCT | SEQ ID NO. 25 |
| | GAATCAAAG | |
| e_nhej_psy1_t2_f | gactGTATCGCCCCT | SEQ ID NO. 26 |
| | GAATCAAAG | |
| f_nhej_psy1_t2_f | ctgaGTATCGCCCCT | SEQ ID NO. 27 |
| | GAATCAAAG | |
| nnn_a_nhej_psy1_ | nnnn | SEQ ID NO. 28 |
| t2_f | nnagtcGTATCG | |
| | CCCCTG AATCAAAG | |
| nnn_b_nhej_psy1_ | nnnnn | SEQ ID NO. 29 |
| t2_f | ntcagGTATCGCCCC | |
| | TGAATCAAAG | |
| nnn_nhej_psy1_t2_r | nnnnn | SEQ ID NO. 30 |
| | ngcctAAATACGGCA | |
| | CTTCCA | |

*n represents A, T, C, or G

Libraries were prepared as Blecher-Gonen et al. (Blecher-Gonen, R. et al. High-throughput chromatin immunoprecipitation for genome-wide mapping of in vivo protein-DNA interactions and epigenomic states. Nat. Protoc. 8, (2013)). High-throughput Sequencing was performed at the G-INCPM unit at the Weizmann Institute of Science with the Illumina HiSeq 2500 platform for 2×125 paired end reads.

DNA samples for Sanger sequencing were amplified using REDTaq® (SIGMA-ALDRICH) with 35 PCR cycles (for primers see Table 7).

TABLE 7
Primer for Sanger Sequencing
Primers for Sanger sequencing of psy1 allele
(SNPs detection)

| | | |
|---|---|---|
| PSY1_1_F | tttgcagaagtca | SEQ ID NO. 31 |
| | agaaacagg | |
| PSY1_t4_ident_R | gatgtcatcgtcc | SEQ ID NO. 32 |
| | gttctcc | |
| PSY1 psnps F | acggtatcttccc | SEQ ID NO. 33 |
| | accttca | |
| PSY1 psnps R2 | atagtgttaattg | SEQ ID NO. 34 |
| | tgtaggctcctt | |
| PSY1 psnps F2 | cgacgaggagtaa | SEQ ID NO. 35 |
| | ggtttgc | |
| PSY1 psnps R | tcagtccatttcg | SEQ ID NO. 36 |
| | ttttcgt | |
| psy1_t123_f | atgttgcagccat | SEQ ID NO. 37 |
| | tcagaga | |
| psy1_t123_r | tgatcatggctcg | SEQ ID NO. 38 |
| | tcactgt | |
| psy1 term f | acaagtaccctgg | SEQ ID NO. 39 |
| | gttggag | |
| psy1_term_r2 | gcagtttttgtag | SEQ ID NO. 40 |
| | gaggcaca | |
| psy1_term_f2 | tgtgcctcctaca | SEQ ID NO. 41 |
| | aaaactgc | |
| psy1 term r | tggattgaatcga | SEQ ID NO. 42 |
| | atttggataa | |
| pimpixm82_co_f | ctttgcacttggt | SEQ ID NO. 43 |
| | tactcaga | |
| pb_psy1_r | agcctacggccca | SEQ ID NO. 44 |
| | aactatt | |
| 14036_f | tgctaatggggca | SEQ ID NO. 45 |
| | ggaaata | |
| 14036_r | tcaagtaacgtaa | SEQ ID NO. 46 |
| | aacacgttgaaa | |
| 5kb_up_t2_F | ttcatttgacgag | SEQ ID NO. 47 |
| | cgatctg | |
| 5kb_up_t2_R | ttggctgctttga | SEQ ID NO. 48 |
| | ccttacc | |
| 40kb_down_t2_f | cattatcctaaga | SEQ ID NO. 49 |
| | gtgcagtcagc | |
| 40kb_down_t2_r | tggtttctcgatt | SEQ ID NO. 50 |
| | acctctttca | |
| 20kb_down_t2_f | tgacaccaatcca | SEQ ID NO. 51 |
| | tccaatc | |
| 20kb_down_t2_r | ctgctacctgcac | SEQ ID NO. 52 |
| | tggctct | |
| 20kb_up_t2_f | tacgtccccgaag | SEQ ID NO. 53 |
| | aaatcac | |
| 20kb_up_t2_r | cccttaggctccg | SEQ ID NO. 54 |
| | aagttgt | |
| 40kb_up_t2_f | cacataagaggac | SEQ ID NO. 55 |
| | acgtttattca | |
| 40kb_up_t2_r | gccacggagaaaa | SEQ ID NO. 56 |
| | tagttga | |

Following PCR, DNA was “cleaned” with Exonuclease I and Shrimp Alkaline Phosphatase (rSAP) (New England BioLabs®). Sequencing was performed at the Biological services unit at the Weizmann Institute of Science with Applied Biosystems 3730 DNA Analyzer.

### Example 1: Tomato Fruit Color Assay for the Analysis of DNA Double-Strand Break (DSB) Repair

Objective: To estimate the rate of somatic non-homologous end joining (NHEJ) versus homologous recombination (HR) based double-strand break (DSB) repair at an endogenous plant locus.

Methods: To estimate the rate of somatic NHEJ versus HR based DSB repair at an endogenous plant locus, a fruit color assay was designed. Two mutant lines of tomato were used, each with a different mutation in the Phytoene synthase 1 (PSY1) gene. The yellow flesh e³⁷⁵⁶ allele is an EMS mutant with a premature stop codon in PSYJ leading to a yellow fruit phenotype (Kachanovsky, D. E., Filler, S., Isaacson, T. & Hirschberg, J. Epistasis in tomato color mutations involves regulation of phytoene synthase 1 expression by cis-carotenoids. Proc. Natl. Acad. Sci. U.S.A 109, 19021-6 (2012)). The bicolor^{cc383} allele is a mutant with a 3.7 Kb deletion in the promoter of PSYJ leading to a yellow-red molted fruit phenotype (FIG. 4A).

In order to monitor the CRISPR-Cas-induced mutations throughout plant development, starting from fertilization, transgenic yellow flesh e³⁷⁵⁶ lines were produced that expressed 35S:Cas9 (SEQ ID NO: 59) and transgenic bicolor^{cc383} lines expressing a PSY1 single guide RNA (u6-26:Ps #1-sgRNA; plasmid sequence is SEQ ID NO: 60; PS #1-sgRNA is SEQ ID NO: 68). This u6-26:Ps #1-sgRNA was designed to induce a DNA DSB between the bicolor^{cc383} and yellow flesh e³⁷⁵⁶ mutations, on both alleles (FIG. 4A). A cross between yellow flesh^{e3756} 35S:Cas9 and bicolor^{cc383} u6-26:Ps #1-sgRNA is expected to yield F₁ plants with the dominant bicolor^{cc383} fruit phenotype. The same is expected for control plants that don't express either 35S:Cas9 or u6-26:Ps #1-sgRNA. Deviations from this phenotype in plants expressing both Cas9 and Ps #1-sgRNA are expected due to the induction of a DSB on one or both alleles followed by error-prone DNA repair. A NHEJ repair of bicolor^{cc383} allele should yield a yellow fruit phenotype (sectors or whole fruit). The outcome of DSB repair by HR based mechanisms (crossover or non-crossover events), should be a red fruit in case of an HR event that occurred early in development, or yellow fruits with red spots or sectors in case of late events (FIG. 4A).

Results: Upon DSB induction, a population of 50 yellow flesh e³⁷⁵⁶ 35 S:Cas9×bicolor^{cc383} u6-26:Ps #1-sgRNA F₁ plants, gave bicolor, yellow and yellow with red spots fruits. The distribution of fruit phenotypes varied when different yellow flesh e³⁷⁵⁶ 35S:Cas9 transgenic lines were used (FIG. 4B). As expected, in the absence of DSB induction, the control population of 6 yellow flesh e³⁷⁵⁶×bicolor^{cc383} F₁ plants, showed only bicolor fruits (FIG. 4B). One of the advantages of the fruit color assay is its ability to predict the inheritance of repair products in the next generation. Indeed it was expected that F₂ seeds extracted from totally yellow fruits would give rise to a germinally transmitted mutation.

To confirm that yellow fruits are indicative of NHEJ germinal events, F₂ plants derived from yellow fruits were grown. Using allele-specific PCR amplification of the yellow flesh e³⁷⁵⁶ and bicolor^{cc383} alleles and sequencing of the PCR products, it was shown that in all cases tested, seeds from yellow fruits yielded seeds carrying a germinally transmitted mutation at the DSB site of the bicolor^{cc383} allele (Table 9). Table 8 presents PCR Primers used.

TABLE 8
Primers for NHEJ Germinal Events
Primers for NHEJ germinal events estimation
(allele specific bends*):

| | | |
|---|---|---|
| JF_F | tgcaaagtgctacgtgtcct | SEQ ID NO. 57 |
| PSY1_1_R | aatgtgaacagcaacgcaaa | SEQ ID NO. 58 |

TABLE 9
NHEJ Germinal Events

| | Fruit | | | | Yellow | Bicolor |
|---|---|---|---|---|---|---|
| F1 plant | number | F2 plant | Cas9 | gRNA | flesh allele | allele |
| 1 | 1 | 11 | − | + | | WT/4 bp del |
| 1 | 1 | 12 | − | + | WT/4 bp del | WT/4 bp del |
| 16 | 1 | 1 | + | + | WT/4 bp del | T insertion |
| 16 | 1 | 2 | − | − | WT | T insertion |
| 16 | 2 | 3 | + | − | WT | T insertion |
| 16 | 2 | 4 | − | + | WT | T insertion |
| 16 | 3 | 1 | + | − | WT | T insertion |
| 16 | 3 | 2 | + | − | | T insertion |
| 18 | 1 | 1 | + | + | | 4 bp del |
| 18 | 1 | 4 | + | + | T insertion | 4 bp del |
| 18 | 2 | 2 | + | + | | 4 bp del |
| 18 | 2 | 3 | − | + | WT/4 bp del | 4 bp del |
| 45 | 1 | 1 | + | − | WT | G insertion |
| 45 | 1 | 6 | + | − | WT | G insertion |
| 45 | 3 | 1 | + | + | WT | G insertion |
| 45 | 3 | 4 | + | − | WT | G insertion |
| 45 | 3 | 5 | + | − | WT | G insertion |

Allele specific PCR products of F₂ plants from yellow fruits of F₁ yellow flesh e³⁷⁵⁶ 35 S:Cas9×bicolor^{cc383} u6-26:Ps #1-sgRNA of Table 9, were sequenced by Sanger sequencing. All the plants had indels (insertions and/or deletions) at the bicolor allele. Four out of 13 had indels in the yellow flesh allele as well.

Some of the progeny from yellow fruits also showed mutation in the yellow flesh e³⁷⁵⁶ allele (Table 9). Although many yellow fruits with small red sectors were found (FIG. 4B), no fully red fruit were detected among the F₁ plants. In addition, a population of 400 F₂ plants derived from fruits with red spots were grown, suggestive of somatic homologous recombination (HR), but no fully red fruit were detected that would indicate germinally transmitted HR events.

### Example 2: High Rate of CRISPR-Cas9 DNA DSB Induction Leads to Repair Via Both Somatic Non-Homologous End-Joining (NHEJ) & Homologous Recombination (HR)

Objective: To identify, characterize and quantify somatic NHEJ events in F₁.

Methods: In order to identify, characterize and quantify somatic NHEJ events in F₁, 22 plants of yellow flesh e³⁷⁵⁶ 35 S:Cas9×bicolor^{cc383} u6-26:Ps #1-sgRNA population were used, and 2 plants of yellow flesh e³⁷⁵⁶×bicolor^{cc383} were used as control. Four (4) leaves from different branches of the plants were used. Then, their DNA was extracted, the region flanking the induced DSB of both alleles was amplified by PCR, and the resulting products were sequenced using high-throughput sequencing Illumina HiSeq 2500 platform.

For the measurement of HR repair, an inverse PCR method was designed that allowed the sequencing of the two allele-specific mutations which are 1.7Kb apart (yellow flesh e³⁷⁵⁶ and bicolor^{cc383}), enabled distinguishing parental from recombinant molecules (FIG. 4D) and minimized the formation of false positive PCR products. The same DNA samples used for the somatic NHEJ sequencing (FIG. 4C) were used for the inverse PCR. In addition, two synthetic positive controls (recombinant-like clones) were cloned that were also treated by the same inverse PCR method. The products of the inverse PCR from each reaction (as shown in FIG. 4D) were sequenced by Illumina HiSeq 2500 paired-end sequencing.

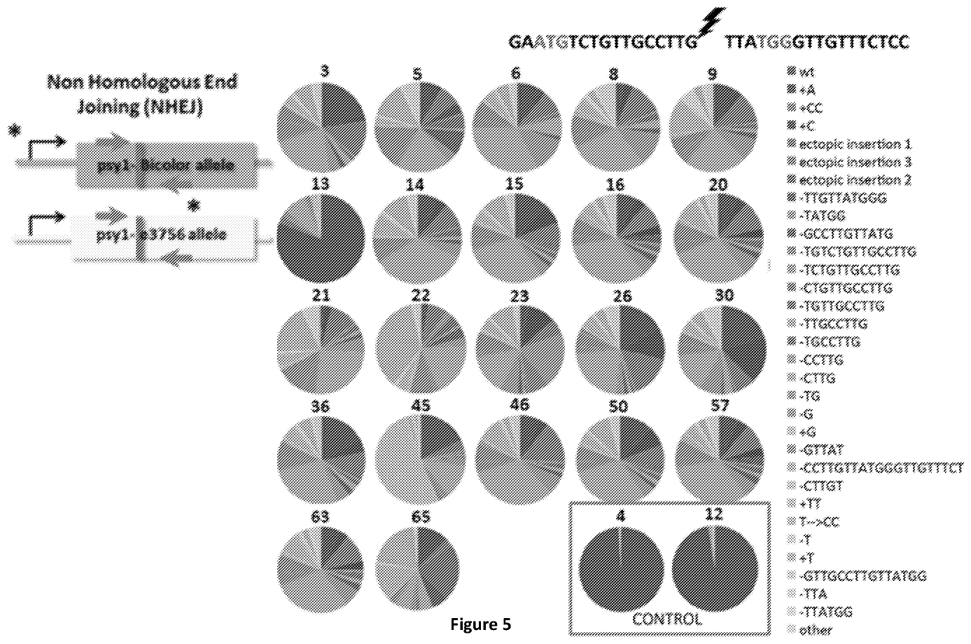

Results: Out of 250,000-850,000 reads per plant (PCR sample), an average of 88% of the reads of yellow flesh e³⁷⁵⁶ 35S:Cas9×bicolor^{cc383} u6-26:Ps #1-sgRNA plants contained a mutation at the CRISPR DSB site, while only 2% of the illumina reads of yellow flesh e³⁷⁵⁶×bicolor^{cc383} plants deviated from the WT sequence, presumably due to PCR and sequencing errors (FIG. 4C). The high rate of CRISPR-Cas DSB induction in the system, lead to a broad spectrum of mutations as a result of different NHEJ repair events. In addition, it was found that some NHEJ signatures, such as the 4 bp CTTG deletion, were preferred over others at this locus (FIG. 4C, FIG. 5).

In the assay measuring HR repair, 5,000-50,000 reads per plant were obtained. The negative controls of yellow flesh e³⁷⁵⁶×bicolor^{cc383} only showed the parental alleles in the absence of DSB induction, while the positive synthetic control showed the recombinant alleles (FIG. 4E). Most F₁ plants of yellow flesh e³⁷⁵⁶ 35S:Cas9×bicolor^{cc383} u6-26:Ps #1-sgRNA showed only the parental alleles but some of them showed one of the recombinant alleles, suggesting somatic HR based repair.

### Example 3: Allele-Specific DSB Induction and High Resolution Analysis of Repair Products

Objective: To create a method that would distinguish between the broken chromosome and the repair template.

The above cross between yellow flesh e³⁷⁵⁶×bicolor^{cc383} did not provide enough SNPs to analyze in detail the HR-repair products. Moreover, it did not enable to perform an allele-specific break, which is needed to perform a precise experiment where it is possible to distinguish between the broken chromosome and the repair template.

Method: Therefore, a new DSB repair assay was designed that provides several SNPs around the break site as well as in distal regions, through the use of Solanum pimpinellifolium^{LA1578}, a wild tomato accession with small red fruits and a sequenced genome showing the presence and location of multiple SNPs compared to Solanum lycopersicum, the edible tomato. In order to ensure allele-specific break, an allele was prepared in the S. lycopersicum M82 cultivar background that is immune to u6-26:Ps #2-sgRNA. For that purpose, the red fruits cv. M82 was transformed with 35S:Cas9 (plasmid sequence is SEQ ID NO: 59; sequence encoding Cas9 is SEQ ID NO: 65) and u6-26:Ps #2-sgRNA (plasmid sequence is SEQ ID NO: 60; sgRNA sequence is encoded by SEQ ID NO: 68). Then, yellow fruits in T₀ were selected for and their T₁ seeds grown. From this T₁ population, a homozygote plant was isolated with an adenine insertion (+A) at the CRISPR-Cas9 DSB site, which was crossed with the wild tomato accession.

The sequence of the gRNA molecule used for the Allele-specific DSB induction and Allele-dependent repair is set forth in SEQ ID NO: 61.

In this assay, the S. pimpinellifolium^{LA1578} is the only target for DNA DSB due to the +A insertion, in the M82 psy1 allele that disrupts the protospacer adjacent motif (PAM) and prevents Cas9 cleavage. The +A mutation of M82 allele is recessive and therefore F₁ plants are expected to have small red fruits. DSB repair in PSY1 by NHEJ, or HR (crossover or non-crossover) leads to yellow fruits or red fruits with yellow sectors, depending on the developmental fruit stage when the repair occurred. NHEJ repair events are expected to leave small indels at the DSB site, while crossover and non-crossover events can be identified by the difference in SNPs patterns on both sides of the DNA DSB (FIG. 6A).

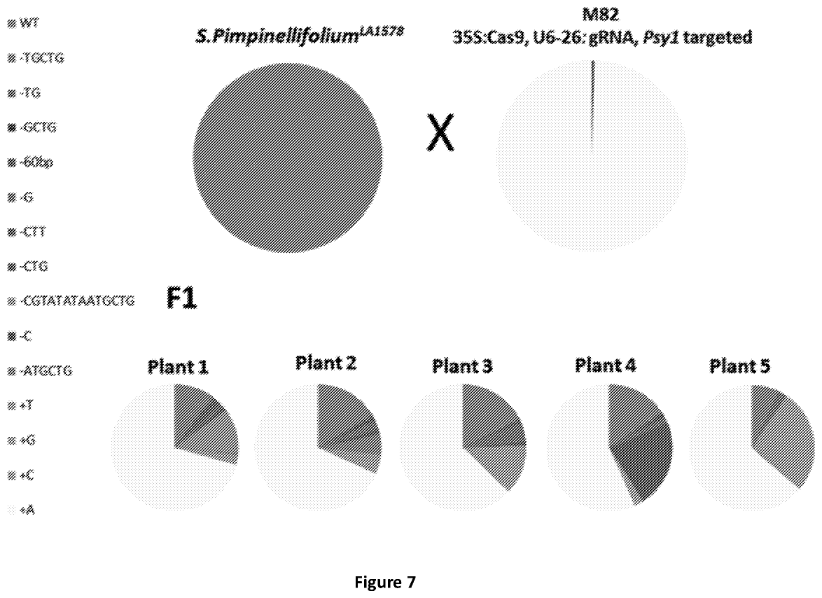

For the analysis of somatic DSB repair, the DSB DNA area from leaves of both parents (M82 35S:Cas9, U6-26:gRNA, +A homozygote and S. pimpinellifolium^{LA1578}) and from five F₁ plants were PCR-amplified and sequenced by illumina HiSeq 2500 paired-end sequencing (FIG. 7). This sequencing yield was 600,000-900,000 reads per plant.

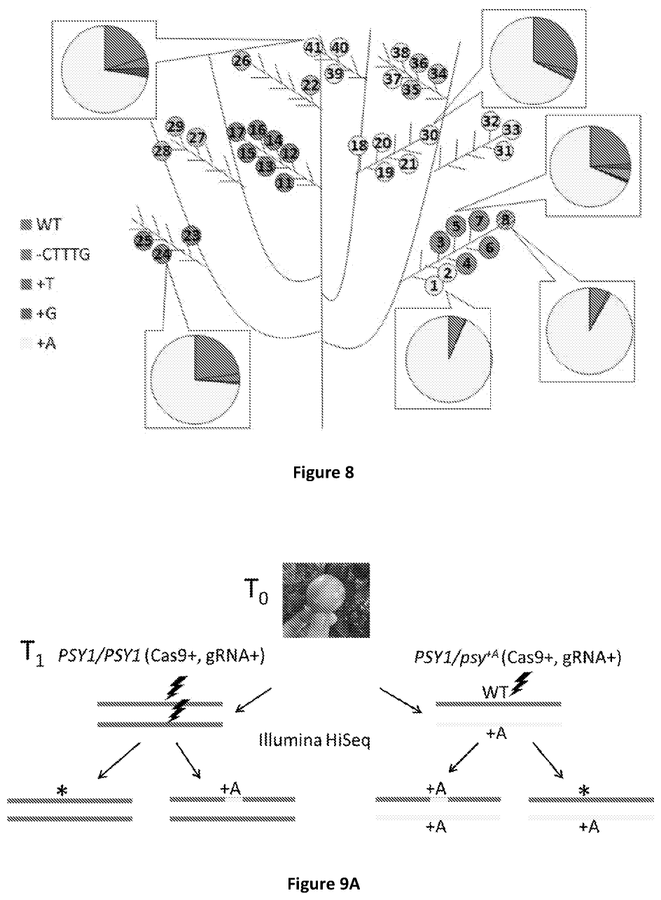

It is shown here that the M82 psy1^{+A} allele was immune to DSB induction, with virtually no DSB footprints in the M82 (M82 35S:Cas9 and u6-26:Ps #2-sgRNA, +A homozygote) parent, supporting the designed allele-specificity of the gRNA (FIG. 7). In addition, at least 50% of the reads gave the +A insertion while the S. pimpinellifolium allele was mutated (red color in the pie chart of FIG. 7). It was found that only 7-18% of the F₁ plants reads were WT and the rest gave various indel patterns (FIG. 7). To estimate the rate of germinal events the fruit colors on different branches were documents and the fruit pericarp tissue was sequenced by illumina (FIG. 8).

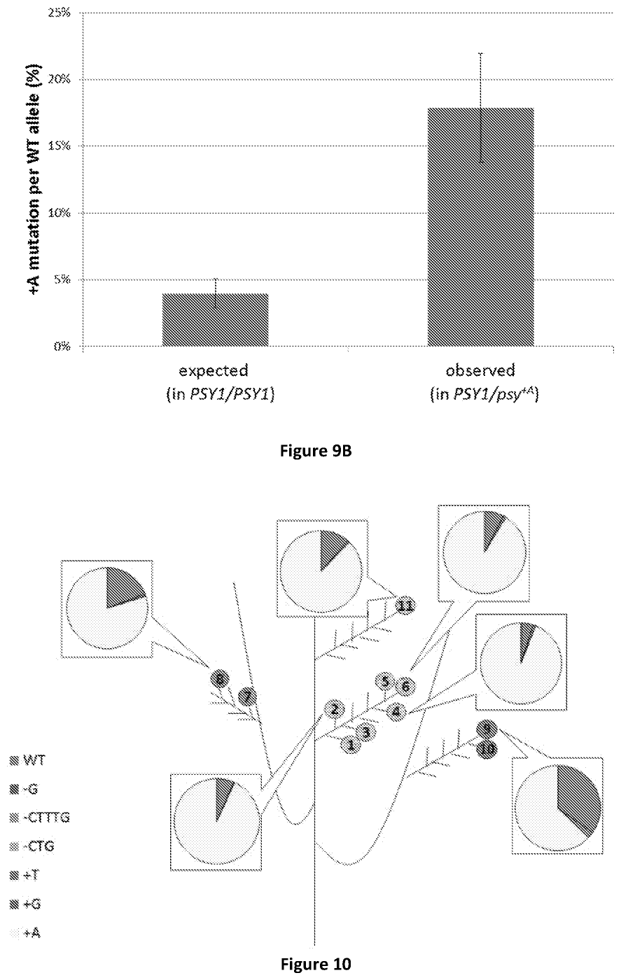

With this assay, the fully yellow fruits might contain seeds that are germinal events of repair via NHEJ or HR (FIG. 6A). Moreover, crossover or non-crossover events should give +A,+A homozygote plant as the repair for template is the M82 psy1^{+A} allele (FIG. 9A). In one of the F₁ Plants, yellow fruits were found that showed high +A,+A content by illumina and Sanger sequencing (FIG. 10). The F₂ progeny of these plants were grown and sequenced by the Sanger method. The sequencing revealed F₂ plants with SNPs patterns corresponding to germinal HR events (FIG. 6B). Plants #2 and #7 look like clear cases of non-crossover, both with conversion tracks of at least 5Kb. Plant #11 looks like a case of crossover (FIG. 6B), however, the analysis of flanking markers (Indels and SNPs), more than 20 kb away from both sides of the DSB site in plant #11 could not be performed due to plant death therefore, so this case was referred to as a putative crossover.

To identify homozygote gene conversion products, and to better characterize the borders of the conversion track, the F₃ plants from the progeny of plant F₂ #7 were sequenced. One of the F₃ progeny of F₂ #7s (Shown at the bottom of FIG. 6B) is an homozygote product of gene conversion repair with a confirmed conversion track of 5-6 kb length.

### Example 4: Quantification of the Rate of Allele-Dependent Repair

Objective: To test allele-dependent repair. Thanks to the system developed above in Example 3 of Allele-specific DSB induction, which is a signature of HR, allele-dependent repair was able to be tested. Induction of DNA DSB on the S. pimpinellifolium^{LA1578} allele showed the +A signature, similar to the M82 psy 1^{+A} allele, at the broken site in many of the fruits and leaves sequenced (FIGS. 7 and 8). This excess in +A repair might be due to preferred NHEJ repair pattern or to allele-dependent repair mediated by HR.

Methods: To distinguish between these two possibilities, several plants of the M82 cultivar were grown, all of them offspring of the same 35S:Cas9 u6-26:Ps #2-sgRNA. In this population, 22 plants were initially homozygote for the M82 WT allele of PSY1, while 14 plants were initially heterozygote M82-WT PSY1/M82 psy1^{+A}. The plants were grown to the age of 4 weeks and 4 leaves collected from each of them. Then, the DNA around the DSB was amplified by PCR and the PCR products were sequenced with the Illumina Hi Seq 2500 platform. For each plant, the percentage of each indel out of the total number of reads was calculated. If the +A mutation occurs independently in each chromosome, there should be twice as many reads with new +A mutations in the WT (which has two potential targets) than in the heterozygote where only one target is available (FIG. 9A).

To measure the expected allele-independent +A NHEJ footprint, the 22 plants of the WT homozygote were used and the percent of +A reads divided by 2 was calculated to obtain the value of the occurrence of the +A mutation per allele. The following equation was used: Expected=(%(+A reads)_{T=4 weeks (wt,wt)})/2. The occurrence of a new +A mutation in the WT allele, when the second allele contains the +A mutation (in M82-WT PSY1/M82 psy1^{+A} heterozygote plants) was calculated by taking the % of +A reads in the M82-WT PSY1/M82 psy1^{+A} plants and deducing 50% (the initial percent of reads originating from the M82 psy1^{+A} allele). The following equation was used for the observed rate of +A mutation in heterozygote M82-WT PSY1/M82 psy1^{+A} plants: Observed=(+A reads)_{T=4 weeks, (wt,+A)}−50%.

Results: When the expected to the observed +A footprint was compared, it was found that there was a significantly higher than expected rate of novel +A mutations in the heterozygote population (p=0.009). Considering that the two populations are isogenic, this suggests that the repair at the site of DSB is dependent on the sequence of its homologous allele (FIG. 9B). The allele-independent frequency of a +A footprint was 4% per allele in the M82-WT while the +A footprint frequency in the M82-WT PSY1 allele in M82-WT PSY1/M82 psy1^{+A} heterozygote was 18% (FIG. 9B). This suggests that 18-4=˜14% of the DSB repair events are allele-dependent (homologous repair recombination events) and the rest occurs via NHEJ in an allele-independent manner.

### Summary for Example 1-4

Somatic DSBs Repair

Earlier studies on somatic DSB-induced HR repair were done mostly with transgenic assays of intrachromosomal recombination or of inter chromatids unequal crossover. Significantly, the methods disclosed and exemplified herein were carried in an endogenous genomic context where the repair template origin could be tracked on the homologous chromosome. The results of Examples 1-4 show that targeted DSBs can be repaired via somatic homologous recombination using a homologous chromosome as the template. This differs significantly from gene targeting using exogenous templates.

In addition, it was demonstrated that some of these repair events can be transmitted germinally to the next generations. In one set of crosses it was shown that the WT allele could be recovered through intragenic recombination between two defective psy1 parental alleles (bicolor^{cc383} and yellow flesh e³⁷⁵⁶), an event seen as red spots (Example 1, FIG. 4A) and characterized through sequence analysis (Example 2, FIG. 4C). In this cross fruits were not recovered that were fully red, and that would correspond to early germinal events. This might be due to the genomic context of the large deletion in the bicolor allele, or alternatively the “cured” recombinant WT allele underwent a second round of NHEJ during development (the target site was not destroyed during HR), that would generate a loss of function (yellow) allele via NHEJ. Considering the high efficiency of NHEJ, this is a plausible scenario. In addition, in an assay of allele-specific DSB induction in a S. pimpinellifolium×S. lycopersicum F₁ hybrid, three cases of HR-dependent repair were found that were germinally transmitted to the F₂ and F₃ generations. Two cases corresponded to non-crossover events with conversion tracks of 5-6 Kb (Example 3, FIG. 6B). The third case (F₂ plant #11) is a germinal HR event that might be either a crossover event or a non-crossover event—this could not be demonstrated due to the plant death. Finally, trying to quantify the ratio of HR versus NHEJ, a sgRNA was designed for allele-specific DSB induction in the S. lycopersicum background. This experimental setup enabled to measure an excess of repair footprints originating from the homologous allele compared to expectation, suggesting that out of all the detectable DSB repair events 14% are allele-dependent and the rest is non-homologous. Fourteen percent allele-dependent HR repair was unexpected, wherein surprisingly the method used produced significantly more HR than was expected.

Somatic Versus Meiotic HR

It is interesting to compare HR-mediated repair in somatic vs. meiotic cells. Overall little is known on inter-homologs recombination in somatic tissues probably owing to the low frequency of such events, to the lack of phenotypic markers and to the difficulty to retrieve germinal events. Red sectors were not detected in the absence of DSBs, and the presence of intragenic recombinant molecules was null or negligible. This is consistent with earlier studies in tobacco showing that the occurrence of somatic HR is very low in the absence of DSB induction for both reciprocal and non-reciprocal HR events. The low rates of somatic HR between homologous chromosomes might be indicative of bottlenecks such as absence of the HR machinery found in meiosis that controls homologs pairing, synaptonemal complex formation, etc. The results, showing an unexpected relatively high rate of HR-repair based on both case-studies and on quantitative assessments, indicate that DSBs are a major bottleneck, surprisingly inducing somatic HR from 0% (in the absence of breaks) to ˜14% per allele (allele-dependent repair measured in Example 4, FIG. 9B) and that DSB-induced HR between homologs can occur in the absence of the meiotic HR machinery.

The rate of HR DSBs repair that is reported here (of ˜14% per allele) appears to be higher than that reported during meiosis. Indeed, only a small fraction of meiotic breaks (˜3-5%) evolves into crossover, and a similar fraction is repaired as non-crossover.

Likewise, evidence is presented on the occurrence of DSB-mediated HR repair, however in most assays it was not possible to distinguish between crossover versus non-crossover repair mechanisms. The analysis of 3 germinal events in a polymorphic background enabled to perform this distinction but the sample (of 2 conversions and one putative crossover) is too small to draw conclusions.

A significant difference compared to earlier meiotic reports, is that the length of conversion tracts in the non-crossover somatic events characterized here ranged ˜5 Kb compared to the mean tract of 552 bp reported for meiotic HR events. These long conversion tracks might reflect a difference between species (tomato versus Arabidopsis) or between meiotic and somatic cells. It could be also that the binding of Spo11 to the DSB ends is more effective than that of Cas9 in protecting ends from degradation and reducing conversion track length. Finally, this is the first report of targeted HR between endogenous homologous chromosomes while there is no earlier report on targeted meiotic recombination.

Somatic crossover does occur in plants, and can even reach high levels in some mutants suggesting that the inter-homolog crossover machinery is available in somatic tissues and that targeted crossover is feasible. Interestingly, even-though the meiotic crossover machinery has been optimized during evolution, the targeted induction of a given DSB during meiosis would have to compete with the hundreds of naturally-occurring other breaks as a substrate for crossover and counter to intuition, might turn out to be less efficient than somatic HR for targeted crossover induction.

Utilization of Somatic HR for Precise Breeding

The results show that custom designed nucleases, such as CRISPR-Cas, may be used for precise reshuffling of chromosomal segments between homologous chromosomes in somatic cells. For example, it might be possible to transfer a disease resistance gene from a wild relative to the crop, without a long process of backcrossing which not only takes several generations in order to achieve isogenic lines, but also drags large segments of undesirable DNA flanking the desirable gene. Thus, use of methods disclosed herein is advantages for use to produce customized recombination in plants with a targeted HR event (crossover or gene conversion) wherein a quality or trait is added or removed from the plant produced compared to a parent plant not undergoing the targeted HR event. In addition, the plant is produced in a reduced time frame and with a significantly smaller population size compared with screening natural recombination events, and the recombination event in the plant of interest is produced more precisely, without also adding undesirable DNA. In some embodiments, the HR crossover or gene conversion event introduces a gene or regulatory element not easily introduced by naturally occurring HR due to tight linkage between genes or gene elements. In other words, methods described and exemplified herein demonstrate that somatic HR can be used for allelic replacement.

### Example 5: Targeted DSB-Induced Crossover in Somatic Tissues in Euchromatin and Heterochromatin Regions in Arabidopsis

Objective: The results of Examples 1-4 above, in tomato, showed high level of HR based repair under somatic DNA DSB. These results were limited to the study of a single locus (PSY1) located in a subtelomeric region of chromosome 3 generally corresponding to euchromatin (open chromatin) regions. Moreover, homology-dependent repair observed could have occurred either by crossover or by gene conversion, as the experimental system did not enable to distinguish between these two mechanisms. Lastly, having studied a single locus in a single species, it was not known how general the phenomenon was and whether the HR-inducing effect of a DSB in somatic tissues would be observed in both euchromatic and heterochromatic (tightly packaged chromatin) regions. The objective here was to examine HR based repair under somatic DNA DSB in another species, and in both euchromatin and heterochromatin regions.

Heterochromatin regions are known to be suppressed in DNA recombination. In some species heterochromatin represents 80% of the genome (e.g. maize and wheat). Heterochromatin is predominant around the centromere and may contain up to 25% of all genes. The lack of recombination in these regions is a hindrance to plant breeding, as deleterious genes cannot be segregated out from the good genes.

Described herein are examples of targeted HR, both crossover and gene conversion, at several genetic loci, including loci with chromatin modifications corresponding to euchromatin (low cytosine methylation, low nucleosome occupancy, Histone3-Lysine4 di or tri methylation (H3K4me2/3)) and loci with heterochromatic features (High cytosine methylation, high nucleosome occupancy, H3K9me2/3, H3K27me3, as would be known in the art). These euchromatin and heterochromatin regions were shown to correspond to meiotic hot spots or cold spots, respectively) (Shilo et al., 2015 “DNA Crossover Motifs Associated with Epigenetic Modifications Delineate Open Chromatin Regions in Arabidopsis” Plant Cell, September; 27(9):2427-36).

Methods: To test the properties of DNA DSB repair in regions with euchromatic and heterochromatic features a crossover tester line was used (Melamed-Bessudo et al. 2005 “A new seed-based assay for meiotic recombination in Arabidopsis thaliana” Plant J. 43(3):458-66), a Columbia ecotype Arabidopsis line with GFP and RFP markers separated by a distance of 5 Mega bp on chromosome 3, expressed under seed specific Napine promoter and giving rise to seeds that are red and green fluorescent (parental types) or red only or green only (crossover recombinant types).

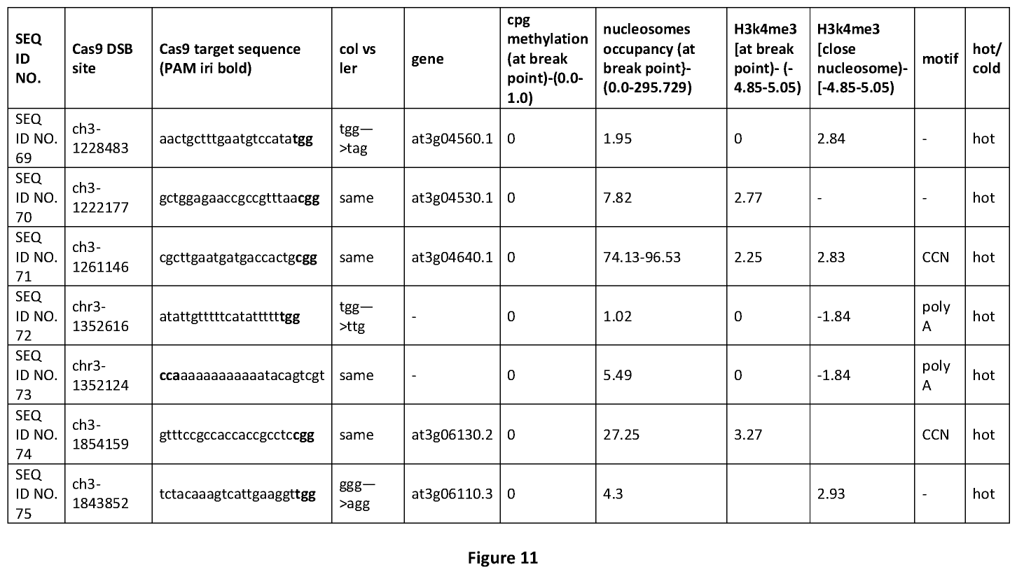

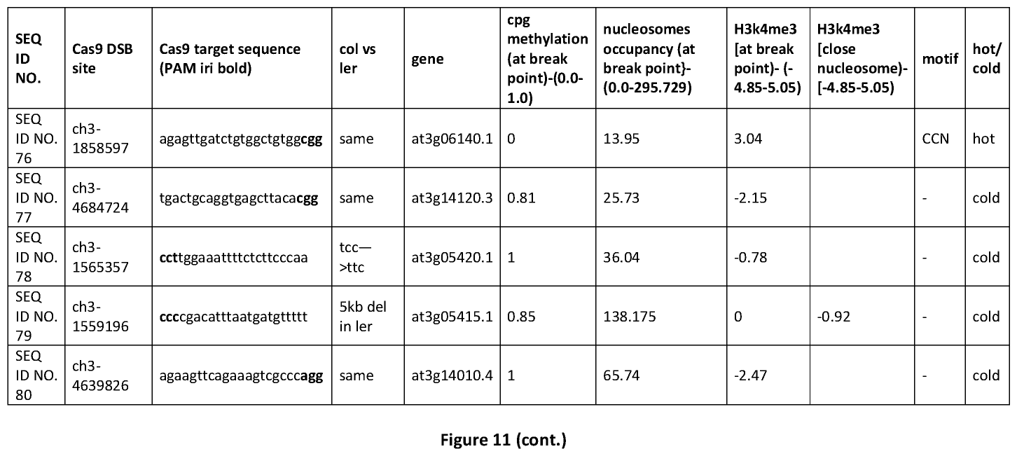

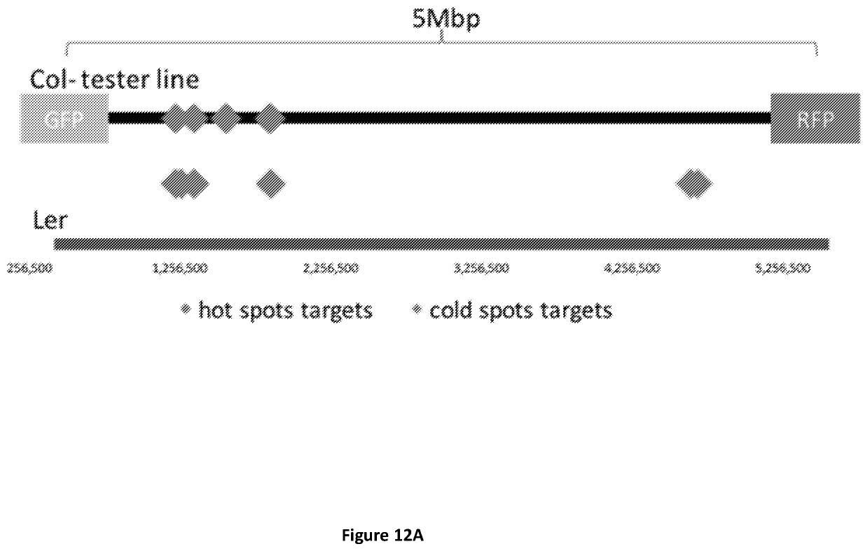

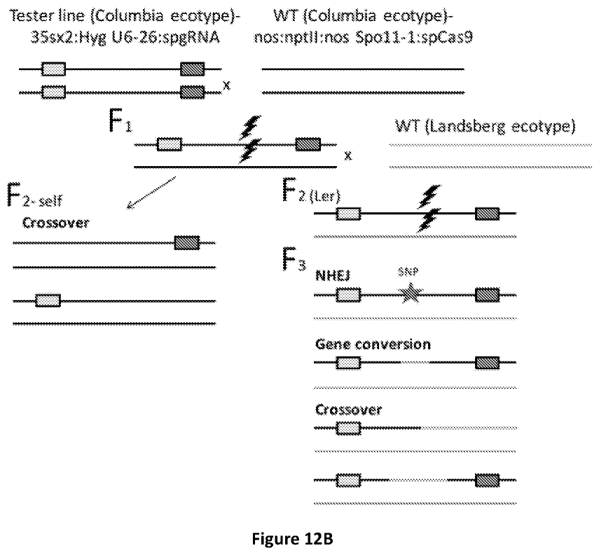

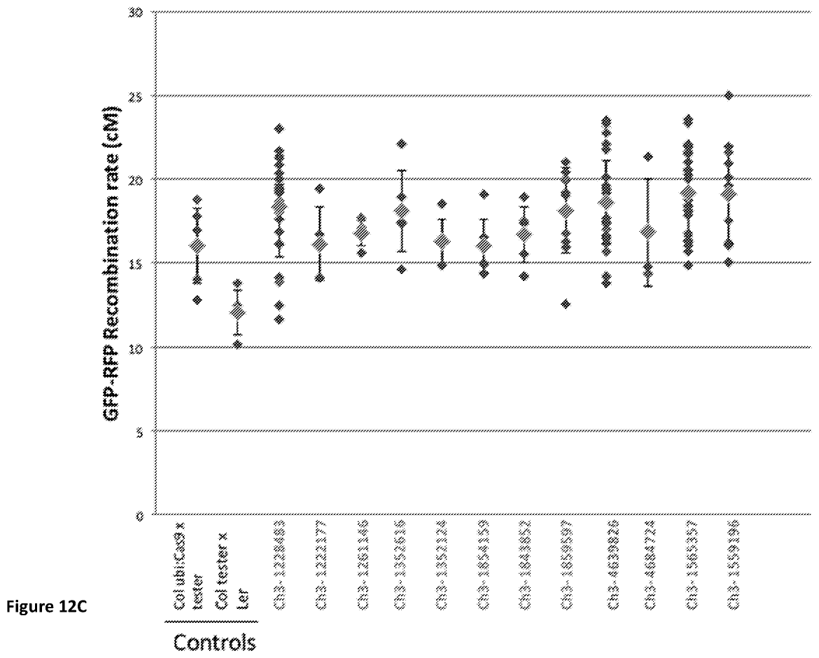

Based on genetic motifs and epigenetics features 12 different targets for DSB induction between the GFP and RFP markers were chosen (Table 10 presented in FIG. 11); four sites in “cold regions” (with heterochromatic features typical of recombination cold spots) and eight targets in “hot regions (euchromatic features typical of recombination hotspots) (FIG. 12A).

First, twelve Columbia recombination tester lines, which included the 12 different targets, were transformed to express the small guide RNAs corresponding to the DSB targets (35Sx2: Hygromycin, u6-26:gRNA construct). In addition, WT Columbia lines were engineered to express Cas9 under a constitutive Ubiquitin promoter active in somatic tissues (nos:nptII:nos Ubi:spCas9). The twelve gRNA-expressing lines were then crossed with the WT Columbia lines expressing Cas9 and F1 plants populations resistant to both hygromycin and Kanamycin (i.e. containing both the gRNA and Cas9) were grown and harvested together with control populations of F1 without gRNA and F1 of Columbia tester line and Landsberg ecotypes (FIG. 12B). In this assay, the DSB break is induced already in somatic tissues and the outcome is measured in seeds. Therefore, early somatic crossover events which are transmitted to the germline will be measured. For each F1 plant, 300-500 of its F2 seeds were counted for red only (recombinant type), green only (recombinant type), red and green (parental type), and non-florescent seeds (parental type). Based on these counts, the recombination frequency between GFP and RFP markers (in cM) were calculated.

Results: Unexpectedly, the results of this test (FIG. 12C) showed that for all hot and cold targets that were counted, a comparable or increased crossover rate was found, relative to the control population of F1 Columbia Ubi:cas9×Columbia tester line control. In this assay both parents were in the Columbia background and only the markers were polymorphic.

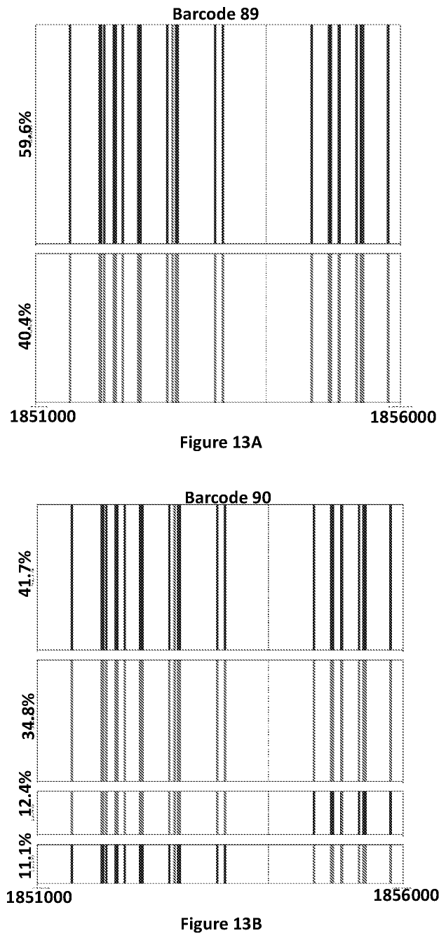

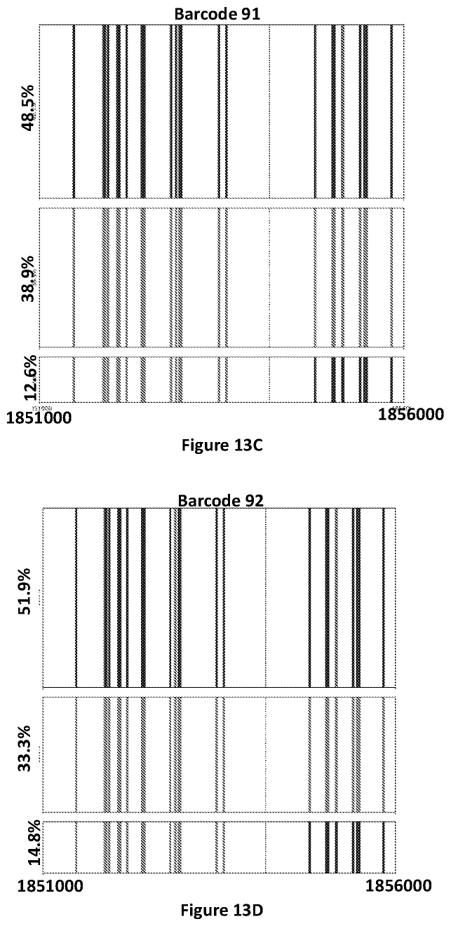

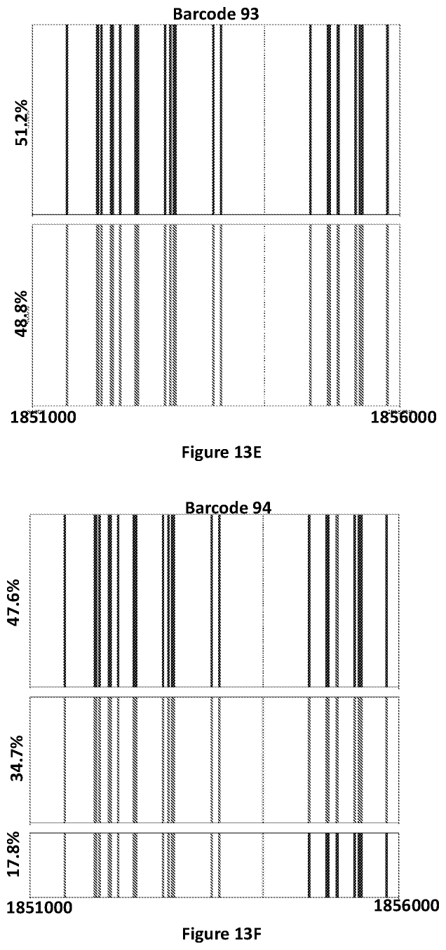

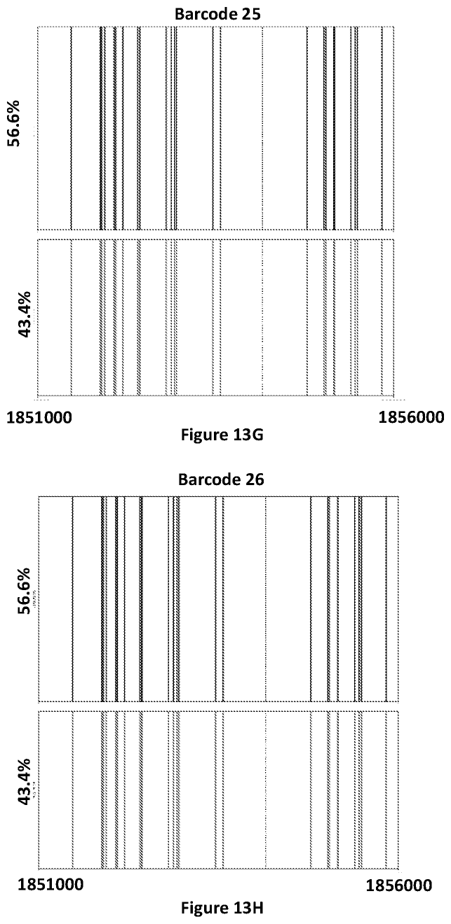

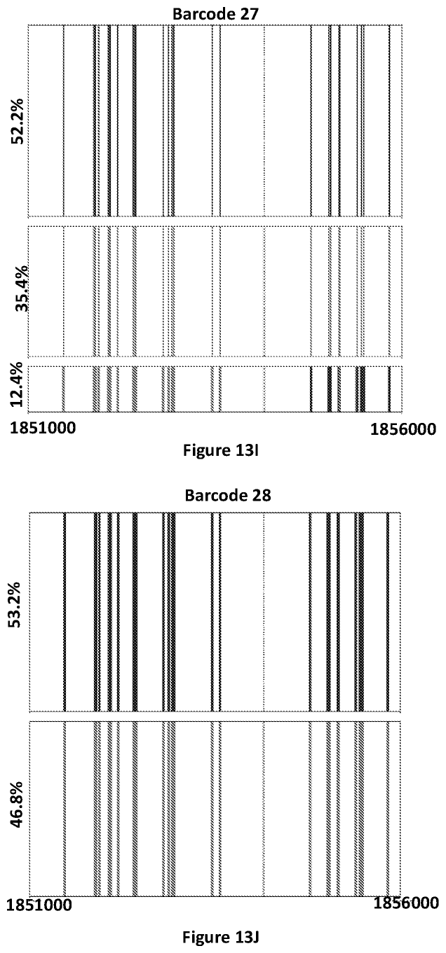

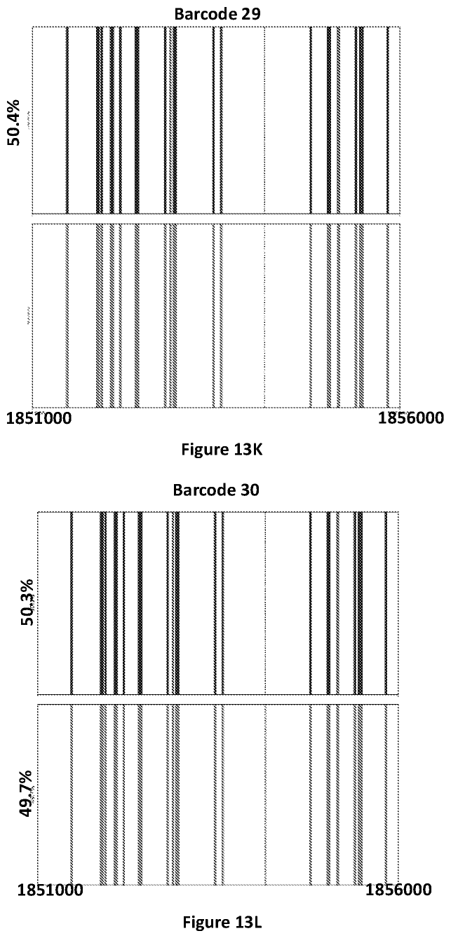

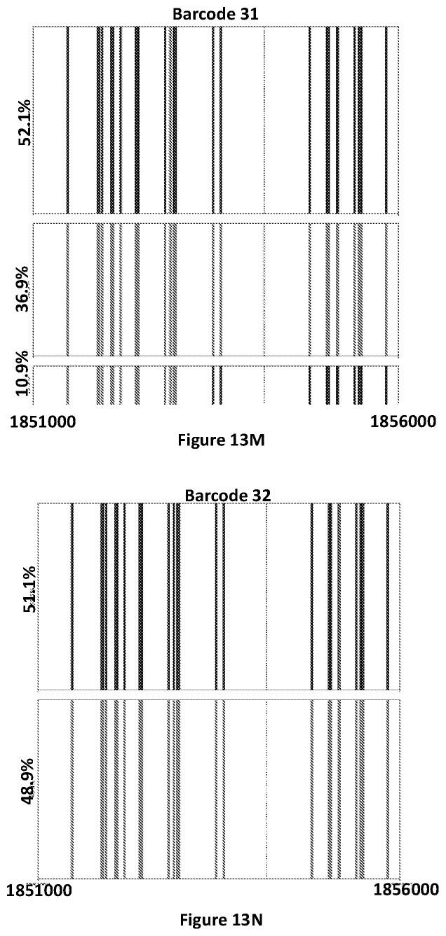

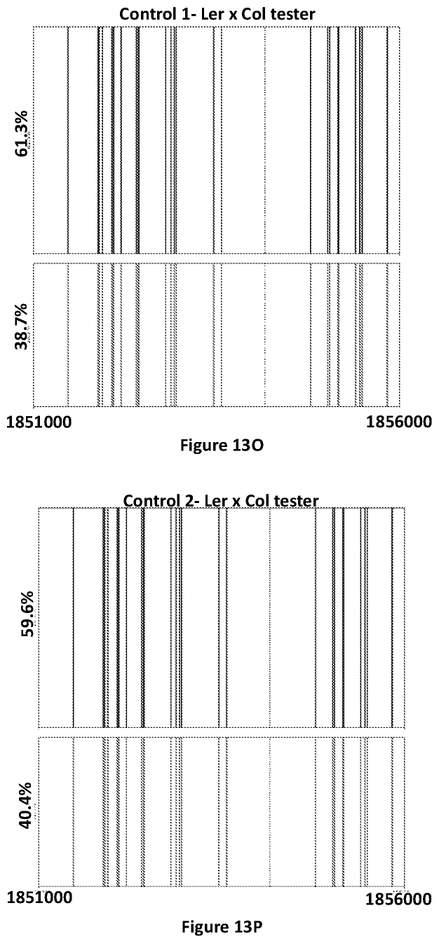

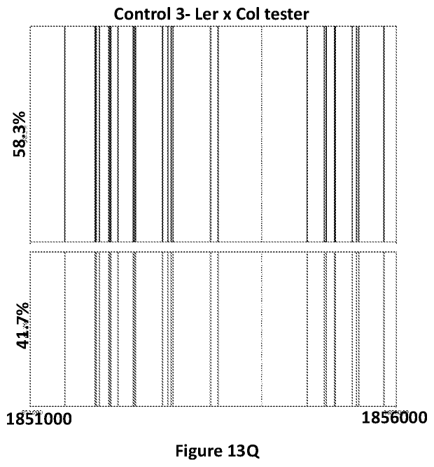

In order to characterize the recombination breakpoints, F1 plants were backcrossed with polymorphic WT Landsberg ecotype, the F2 backcross populations (hygromycin and kanamycin resistant plants) were grown, DNA was extracted from the somatic tissues of these plants, and their F3 seeds were collected (FIG. 12B). Using PacBio, 5Kb fragments flanking the targeted area of these backcrossed plants of Columbia tester×Landsberg, were sequenced for high resolution characterization of DNA DSB repair events (FIG. 13). Overall these results provide support for targeted DSB-induced recombination at both euchromatin and heterochromatin repair sites.

While certain features disclosed herein have been illustrated and described herein, many modifications, substitutions, changes, and equivalents will now occur to those of ordinary skill in the art. It is, therefore, to be understood that the appended claims are intended to cover all such modifications and changes as fall within the true spirit of the genetically modified plants and methods disclosed herein.

What is claimed is:

1. A method of targeting DNA recombination between homologous chromosomes in a somatic plant cell, said method comprising the steps of:
(a) expressing a nuclease system in said plant cell, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles;
(b) analyzing progeny of said plant cell, or a plant tissue grown from said plant cell, or a plant grown from said cell or a progeny of said plant thereof, for homologous recombination between the homologous chromosomes, wherein said homologous recombination comprises crossover or gene conversion (non-crossover); and
(c) selecting a plant cell, plant tissue thereof, plant thereof, or plant progeny thereof wherein targeted homologous recombination has occurred.

2. The method of claim 1, wherein said nuclease system comprises
(a) a zinc finger nuclease (ZFN) system comprising a zinc finger nuclease (ZFN) comprising a zinc-finger DNA binding domain and a DNA nuclease cleavage domain, wherein said zinc-finger DNA binding domain binds within said preselected endogenous target site, thereby targeting the DNA nuclease cleavage domain to cleave the DNA within said preselected endogenous target site; or
(b) a transcription activator-like effector nuclease (TALEN) system comprising a TAL effector DNA binding domain and a DNA cleavage domain, wherein said TAL effector DNA binding domain binds within said preselected endogenous target site, thereby targeting the DNA cleavage domain to cleave the DNA within said preselected endogenous target site; or
(c) a clustered regularly interspaced short palindromic repeats (CRISPR)/CRISPR associated proteins (Cas) system comprising a CRISPR-associated endonuclease and a gRNA molecule, wherein said gRNA molecule binds within said preselected endogenous target site thereby guiding said CRISPR-associated endonuclease to cleave the DNA within said preselected endogenous target site.

3. (canceled)

4. (canceled)

5. (canceled)

6. The method of claim 2, wherein said CRISPR-associated endonuclease (Cas nuclease) is selected from the group comprising Cas1, Cas1B, Cas2, Cas3, Cas4, Cas5, Cas6, Cas7, Cas8, Cas9, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, C2c1, CasX, NgAgo, Csf1, Csf2, Csf3, and Csf4, homologs thereof, or modified versions thereof.

7. The method of claim 1, wherein said somatic plant cell originates from
(a) an existing hybrid or heterozygous plant cell having polymorphic alleles at said preselected site; or
(b) a cell from the progeny of crossing two plants, wherein said parental plant cells each comprise a polymorphic allele compared with said mate at said preselected site.

8. The method of claim 7, wherein when said somatic plant cell originates from an existing hybrid or heterozygous,
(a) said existing hybrid or heterozygous plant cell originates from a wild-type plant; or
(b) said method produces a somatic plant cell comprising a targeted homologous recombination within the preselected endogenous target site, or a plant tissue comprising said somatic plant cell, or a plant comprising said somatic plant cell or a progeny plant thereof or fruit derived from a plant comprising said somatic plant cell or progeny plant thereof, or seeds derived from a plant comprising said somatic plant cell or progeny plant thereof or any combination thereof, having a combination of parental traits, said combination not present in either parent: or
(c) any combination thereof.

9. (canceled)

10. The method of claim 9, wherein said parental traits comprise increased drought resistance, increased resistance to pests, increased resistance to pathogens, improved nutrient content, or improved growth parameters, or any other trait of benefit to the plant cell, plant tissue, plant, fruit, or seed.

11. (canceled)

12. The method of claim 7, when said somatic plant cell originates from a cell from the progeny of crossing two plants, said method produces a somatic plant cell comprising a targeted homologous recombination within the preselected endogenous target site, or a plant tissue comprising said somatic plant cell, or a plant comprising said somatic plant cell or a progeny plant thereof, or fruit derived from a plant comprising said somatic plant cell or progeny plant thereof, or seeds derived from a plant comprising said somatic plant cell or progeny plant thereof, or any combination thereof, having a resultant combination of parental traits said combination not present in either parent.

13. The method of claim 12, wherein said parental traits recombined through said targeted homologous recombination comprise increased drought resistance, increased resistance to pests, increased resistance to pathogens, improved nutrient content, or improved growth parameters, or any other trait of benefit to the plant cell, plant tissue, plant, fruit, or seed.

14. The method of claim, when said somatic plant cell originates from a cell from the progeny of crossing two plants
(a) one of said parent somatic plant cells comprises said nuclease system, and wherein the DNA cleaving activity of said nuclease system is targeted to the polymorphic allele present in the other parent plant cell that does not comprise said nuclease system; or
(b) one of said parent somatic plant cells comprises a Cas nuclease and the other of said parent somatic plant cells comprises a gRNA molecule, wherein said gRNA molecule binds within said preselected endogenous target site thereby guiding said Cas nuclease to cleave the DNA within said preselected endogenous target site.

15. (canceled)

16. The method of claim 1, wherein said somatic plant cell comprises a cell from a plant progeny of a cross between two polymorphic parental lines, which creates a hybrid plant, wherein said parental plant lines each comprise a polymorphic allele at said preselected endogenous target site, and wherein only one of the parental lines comprises said nuclease system.

17. The method of claim 16, wherein
(a) said method produces a somatic plant cell comprising a targeted homologous recombination within the preselected endogenous target site, or a plant tissue comprising said somatic plant cell, or a plant comprising said somatic plant cell or a progeny plant thereof, or fruit derived from a plant comprising said somatic plant cell or progeny plant thereof, or seeds derived from a plant comprising said somatic plant cell or progeny plant thereof, or any combination thereof, having a combination of parental traits said combination not present in either parent; or
(b) said nuclease system comprises a Cas nuclease and a gRNA molecule, wherein said gRNA molecule binds within said preselected endogenous target site thereby guiding said Cas nuclease to cleave the DNA within said preselected endogenous target site, and wherein the DNA cleaving activity of said nuclease system occurs solely on the heterologous allele present in wild-type parent plant cell; or
(c) a combination of (a) and (b).

18. The method of claim 17, wherein said parental traits comprise increased drought resistance, increased resistance to pests, increased resistance to pathogens, improved nutrient content, or improved growth parameters, or any other trait of benefit to the plant cell, plant tissue, plant, fruit, or seed.

19. (canceled)

20. The method of claim 1, wherein
(a) said somatic plant cell is comprised within a plant tissue or a whole plant; or
(b) said preselected endogenous target site comprises DNA comprising a gene, a part of a gene, or a regulatory upstream or downstream sequences of a gene, or any combination thereof, and wherein expression or lack thereof of said gene affects growth, drought resistance, resistance to pests, resistance to pathogens, or nutrient content, or any other trait of benefit to the plant cell, plant tissue, plant, fruit, or seed, or any combination thereof; or
(c) the preselected endogenous target site comprises a region of euchromatin or heterochromatin; or
(d) said expression comprises constitutive induction of expression, inducible induction of expression, tissue-specific induction of expression, or condition-specific induction of expression, or any combination thereof; or
(e) any combination of (a), (b), (c), and (d).

21. (canceled)

22. (canceled)

23. (canceled)

24. The method of claim 1, wherein
(a) said somatic plant cell comprises a protoplast; or
(b) said somatic plant cell comprises a crop plant cell; or
(c) said selected progeny of step (d) comprise F1, F2, or F3 generations, or any subsequent generation, or backcrosses for 1 to 3 generations, or any subsequent backcross generation; or
(d) any combination of (a), (b), and (c).

25. (canceled)

26. The method of claim 1, wherein analyzing said plant comprises analyzing a portion of said plant or a progeny thereof comprising a leaf, a stem, a bud, a fruit, a seed.

27. (canceled)

28. The method of claim 1, wherein said method produces a somatic plant cell comprising said targeted homologous recombination at said preselected endogenous target site, or a plant tissue comprising said targeted homologous recombination at the preselected endogenous target site, or a plant comprising said targeted homologous recombination at the preselected endogenous target site or a progeny plant thereof, or fruit derived from a plant comprising targeted homologous recombination at the preselected endogenous target site or progeny plant thereof, or seeds derived from a plant comprising said targeted homologous recombination at the preselected endogenous target site or progeny plant thereof, or any combination thereof, said cell, tissue, plant or progeny thereof, fruit, or seed comprising genes for increased drought resistance, increased resistance to pests, increased resistance to pathogens, improved nutrient content, improved growth parameters, or any other trait of benefit to the plant cell, plant tissue, plant or progeny thereof, fruit, or seed, or any combination thereof as compared to a control plant cell, plant or progeny thereof, fruit, or seed.

29. A plant comprising a combination of beneficial traits or qualities produced by a method comprising targeted DNA recombination between homologous chromosomes in a hybrid somatic plant cell, said method comprising the steps of:
(a) expressing a nuclease system in said plant cell, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles;
(b) analyzing progeny of said plant cell, or a plant tissue grown from said plant cell, or a plant grown from said cell or a progeny of said plant thereof, for homologous recombination between the homologous chromosomes, wherein said homologous recombination comprises crossover or gene conversion (non-crossover);
(c) selecting a plant cell, plant tissue thereof, plant thereof, or plant progeny thereof wherein targeted homologous recombination has occurred;
(d) propagating said plant cell, or plant tissue thereof, or plant thereof, or plant progeny thereof to produce a plant comprising said targeted homologous recombination, wherein said plant comprises a combination of beneficial qualities or traits not present in either parent plant from which the hybrid somatic cell originated.

30. The plant of claim 29, wherein the preselected endogenous target site comprises a region of euchromatin or heterochromatin.

31. A method of producing a progeny plant comprising a combination of beneficial traits or qualities, wherein said combination is not present in either parent plant, said method comprising:
(a) selecting parent plants, wherein each of said parents comprises at least one beneficial trait, wherein said beneficial traits are not identical and wherein said parents are polymorphic for one said at least beneficial trait;
(b) crossing said parent plants to creates a hybrid plant;
(c) collecting somatic cells from the hybrid plant;
(d) expressing a nuclease system in said somatic cells, wherein said expressed nuclease system is targeted to a preselected endogenous target site comprising polymorphic alleles on the homologous chromosomes, wherein upon expression of said nuclease system the DNA of at least one of said polymorphic allele is cleaved within said preselected endogenous target site, wherein said nuclease cleaves the DNA creating a double-strand break in the DNA of at least one of said polymorphic alleles, wherein homologous crossover or gene conversion (non-crossover) at said targeted preselected endogenous target site leads to an exchange of DNA expressing or regulating the expression of at least one of said beneficial traits or qualities;
(e) analyzing progeny of said plant cells, or a plant tissue grown from said plant cells, or a plant grown from said cells or a progeny of said plant thereof, for said crossover or gene conversion (non-crossover) event wherein said combination of traits is expressed;
(f) selecting a plant cell, plant tissue thereof, plant thereof, or plant progeny thereof wherein the combination of traits is expressed; and
(g) propagating said plant cell, plant tissue thereof, plant thereof, to produce a progeny plant that comprise said combination of beneficial traits or qualities.

32. The method of claim 31, wherein the preselected endogenous target site comprises a region of euchromatin or heterochromatin.
